(19) United States
(12) Patent Application Publication
(10) Pub. No.: US 2009/0099213 A1
(43) Pub. Date: Apr. 16, 2009

(54) Ortho-Condensed Pyridine and Pyrimidine Derivatives (e.g., Purines) as Protein Kinases Inhibitors

(75) Inventors: Valerio Berdini, Cambridge (GB); Robert George Boyle, Cambridge (GB); Gordon Saxty, Cambridge (GB); Marinus Leendert Verdonk, Cambridge (GB); Steven John Woodhead, Cambridge (GB); Paul Graham Wyatt, Perth (GB); Hannah Fiona Sore, Cambridge (GB); John Caldwell, Sutton (GB); Ian Collins, Sutton (GB); Tatiana Faria Da Fonseca, Sutton (GB); Alastair Donald, Oxfordshire (GB)

Correspondence Address: HESLIN ROTHENBERG FARLEY & MESITI PC, 5 COLUMBIA CIRCLE, ALBANY, NY, 12203, US

(73) Assignees: Astex Therapeutics Limited, Cambridge (GB); The Institute of Cancer Research: Royal Cancer Hospital, London (GB); Cancer Research Techology Limited, London (GB)

(21) Appl. No.: 11/577,962

(22) PCT Filed: Oct. 25, 2005
(86) PCT No.: PCT/GB05/04115

§ 371 Date: Aug. 5, 2008

Related U.S. Application Data
(60) Provisional application No. 60/621,719, filed on Oct. 25, 2004.
(60) Provisional application No. 60/683,980, filed on May 24, 2005.

(30) Foreign Application Priority Data
Oct. 25, 2004 (GB) 0423684.0

(51) Int. Cl. A61K 31/52; A61P 35/00; C07D 473/34

(52) U.S. Cl. 514/263.22; 514/263.4; 514/263.23; 544/277

(57) ABSTRACT

The invention provides a compound for use in the prophylaxis or treatment of a disease state or condition mediated by protein kinase B, the compound having the formula (I): or salts, solvates, tautomers or N-oxides thereof, wherein T is N or CR⁵; J¹-J² is N═C(R⁶), (R⁷)C═N, (R⁸)N—C(O), (R⁸)₂C—C(O), N═N or (R⁷)C═C(R⁶); A is an optionally substituted saturated C₁₋₇ hydrocarbon linker group having a maximum chain length of 5 atoms extending between R¹ and NR²R³ and a maximum chain length of 4 atoms extending between E and NR²R³, one of the carbon atoms in the linker group being optionally replaced by oxygen or nitrogen; E is a monocyclic or bicyclic carbocyclic or heterocyclic group or an acyclic group X-G wherein X is CH₂, O, S or NH and G is a C₁₋₄ alkylene chain wherein one of the carbon atoms is optionally replaced by O, S or NH; R¹ is hydrogen or an aryl or heteroaryl group; R² and R³ are each hydrogen, optionally substituted C₁₋₄ hydrocarbyl or optionally substituted C₁₋₄ acyl; or NR²R³ forms an imidazole group or a saturated monocyclic heterocyclic group having 4-7 ring members; or NR²R³ and A together form a saturated monocyclic heterocyclic group having 4-7 ring members which is optionally substituted by C₁₋₄alkyl; or NR²R³ and the adjacent carbon atom of linker group A together form a cyano group; or R¹, A and NR²R³ together form a cyano group; and R⁴, R⁵, R⁶, R⁷ and R⁸ are each independently selected from hydrogen and various substituents as defined in the claims.

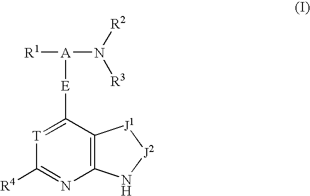

### RELATED APPLICATIONS

This application is related to U.S. provisional patent applications U.S. 60/621,719 (filed 25 Oct. 2004) and U.S. 60/683,980 (filed 24 May 2005), the contents of each of which are incorporated herein by reference.

### TECHNICAL FIELD

This invention relates to purine, purinone and deazapurine and deazapurinone compounds that inhibit or modulate the activity of protein kinase B (PKB) and protein kinase A (PKA), to the use of the compounds in the treatment or prophylaxis of disease states or conditions mediated by PKB and PKA, and to novel compounds having PKB and PKA inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzymes of the PI3K family are activated by a range of growth and survival factors e.g. EGF, PDGF and through the generation of polyphosphatidylinositols, initiates the activation of the downstream signalling events including the activity of the kinases PDK1 and protein kinase B (PKB) also known as akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKBₐₗₚₕₐ (akt1) itself is phosphorylated on Thr 308 by PDK1 and on Ser 473 by a kinase referred to as PDK2, whereas PKB_{beta} (akt2) is phosphorylated on Thr 309 and on Ser 474, and PKB_{gamma} (akt3) is phosphorylated on Thr 305 and on Ser 472.

At least 10 kinases have been suggested to function as a Ser 473 kinase including mitogen-activated protein (MAP) kinase-activated protein kinase-2 (MK2), integrin-linked kinase (ILK), p38 MAP kinase, protein kinase Calpha (PKCalpha), PKCbeta, the NIMA-related kinase-6 (NEK6), the mammalian target of rapamycin (mTOR), the double-stranded DNA-dependent protein kinase (DNK-PK), and the ataxia telangiectasia mutated (ATM) gene product. Available data suggest that multiple systems may be used in cells to regulate the activation of PKB. Full activation of PKB requires phosphorylation at both sites whilst association between PIP3 and the PH domain is required for anchoring of the enzyme to the cytoplasmic face of the lipid membrane providing optimal access to substrates.

Activated PKB in turns phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{Cip1/WAF1}, and phosphorylation and activation of mTOR, a kinase controlling several aspects of cell size, growth and protein translation.

The phosphatase PTEN which dephosphorylates and inactivates polyphosphatidylinositols is a key tumour suppressor protein which normally acts to regulate the PI3K/PKB survival pathway. The significance of the PI3K/PKB pathway in tumourigenesis can be judged from the observation that PTEN is one of the most common targets of mutation in human tumours, with mutations in this phosphatase having been found in 50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma, which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function. Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interferon gamma activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta isoform primarily. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits.

PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation. Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy.—Aandahl, E. M., Aukrust, P., Skåalhegg, B. S., Müiller, F., Frøland, S. S., Hansson, V., Taskén, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases derived from this, such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients. Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

### PRIOR ART

Several classes of compounds have been disclosed as having PKA and PKB inhibitory activity. For example, a class of isoquinolinyl-sulphonamido-diamines having PKB inhibitory activity is disclosed in WO 01/91754 (Yissum).

WO 93/13072 (Italfarmaco) discloses a class of bis-sulphonamido diamines as protein kinase inhibitors.

Purines and purine analogues and derivatives have been disclosed as having a wide range of different biological activities.

For example, WO03/057696 (Eisai) discloses a class of indolyl-deazapurines for treating inflammatory or autoimmune or proliferative diseases.

WO 99/65909 (Pfizer) discloses a class of pyrrole[2,3-d pyrimidine compounds as inhibitors of protein tyrosine kinases such as Janus kinase 3. The compounds are described as having a range of therapeutic uses.
Semonsky et al. Czech. Chem. Comm. (1960), 25, 1091-1099, disclose derivatives of 6-carboxyalkylthiopurine as anti-cancer agents.
Noell et al., J. Org. Chem., (1958), 23, 1547-1550 disclose 4-(substituted amino)pyrazole[3,4-d]pyrimidines as potential anti-tumour agents.
Lettre et al., Naturwissenschaften (1958), 45, 364 disclose several aminoalkyl-aminopurine derivatives having activity against tumour cells.
US 2003/0139427 (OSI) discloses pyrrolidine- and piperidine-substituted purines and purine analogues having adenosine receptor binding activity. WO 2004/043380 (Harvard College et al.) discloses technetium and rhenium labelled imaging agents containing disubstituted piperidine metal ion-chelating ligands.

WO 97/38665 (Merck) discloses gem-disubstituted piperidine derivatives having farnesyl transferase inhibitory activity.

EP 1568699 (Eisai) discloses 1,3-dihydroimidazole fused ring compounds having DPPIV-inhibiting activity. The compounds are described as having a range of potential uses including the treatment of cancer.

US 2003/0073708 and US 2003/045536 (both in the name of Castelhano et al), WO 02/057267 (OSI Pharmaceuticals) and WO 99/62518 (Cadus Pharmaceutical Corporation) each disclose a class of 4-aminodeazapurines in which the 4-amino group can form part of a cyclic amine such as azetidine, pyrrolidine and piperidine, The compounds are described as having adenosine receptor antagonist activity.

U.S. Pat. No. 6,162,804 (Merck) discloses a class of benzimidazoles and imidazopyridines as tyrosine kinase inhibitors.

### SUMMARY OF THE INVENTION

The invention provides compounds that have protein kinase B (PKB) and/or protein kinase A (PKA) inhibiting or modulating activity, and which it is envisaged will be useful in preventing or treating disease states or conditions mediated by PKB and/or PKA.

Accordingly, in a first aspect, the invention provides, for use in the prophylaxis or treatment of a disease state or condition mediated by protein kinase B, a compound of the formula (I):

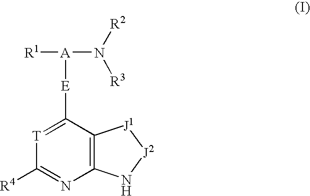

or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

In another aspect, the invention provides, for use in the prophylaxis or treatment of a disease state or condition mediated by protein kinase B, a compound of the formula (Ia):

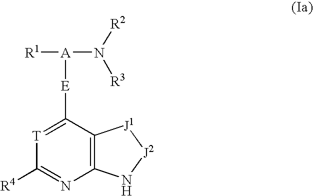

or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom α with respect to the NR²R³ group and provided that the oxo group when present is located at a carbon atom a with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl;
or R² and R³ together with the nitrogen atom to which they are attached form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

In a further aspect, the invention provides a compound of the formula (Ib):

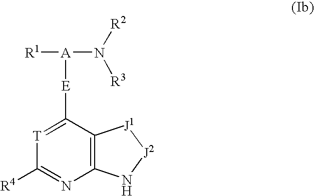

or salts, solvates, tautomers or N-oxides thereof, wherein
T is N or a group CR⁵;
J¹ J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
   provided that:
   (a-i) when J¹-J² is (R⁷)C═C(R⁶) and E is a monocyclic or bicyclic group linked through a nitrogen atom to the ring containing T, then A contains no oxo substituent;
   (a-ii) E is other than an unsubstituted or substituted indole group;
   (a-iii) when J¹-J² is N═CH, then E-A(R¹)—NR²R³ is other than a group —S—(CH₂)₃—CONH₂ or —S—(CH₂)₃—CN;
   (a-iv) when J¹-J² is CH═N, then E-A(R¹)—NR²R³ is other than a group —NH—(CH₂)ₙ—N(CH₂CH₃)₂ where n is 2 or 3; and
   (a-v) when J¹-J² is N═CH, then E-A(R¹)—NR²R³ is other than a group —NH—(CH₂)₂—NH₂ or —NH—(CH₂)₂—N(CH₃)₂.

In another aspect, the invention provides a compound of the formula (Ic):

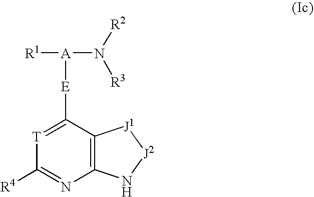

or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from fluorine and hydroxy, provided that the hydroxy group when present is not located at a carbon atom a with respect to the NR²R³ group;
E is a monocyclic carbocyclic or heterocyclic group;
R¹ is an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

In further aspects, the invention provides:
A compound per se of the formula (Ia), (Ib), (Ic), (II), (IIa), (IIb), (III) or any other sub-group or embodiment of the formula (I) as defined herein.
A compound of the formula (I), (Ia), (Ib), (Ic), (II), (IIa), (IIb), (III) or any sub-group or embodiment thereof as defined herein for use in the prophylaxis or treatment of a disease state or condition mediated by protein kinase B.
The use of a compound of formula (I), (Ia), (Ib), (Ic), (II), (IIa), (IIb), (III) or any sub-group or embodiment thereof as defined herein for the manufacture of a medicament for the prophylaxis or treatment of a disease state or condition mediated by protein kinase B.
A method for the prophylaxis or treatment of a disease state or condition mediated by protein kinase B, which method comprises administering to a subject in need thereof a compound of the formula (I), (Ia), (Ib), (Ic), (II), (IIa), (IIb), (III) or any sub-group or embodiment thereof as defined herein.
A method for treating a disease or condition comprising or arising from abnormal cell growth or abnormally arrested cell death in a mammal, the method comprising administering to the mammal a compound of the formula (I), (Ia), (Ib), (Ic), (II), (IIa), (IIb), (III) or any sub-group or embodiment thereof as defined herein in an amount effective to inhibit protein kinase B activity.
A method of inhibiting protein kinase B, which method comprises contacting the kinase with a kinase-inhibiting compound of the formula (I), (Ia), (Ib), (Ic), (II), (IIa), (IIb), (III) or any sub-group or embodiment thereof as defined herein.
A method of modulating a cellular process (for example cell division) by inhibiting the activity of a protein kinase B using a compound of the formula (I), (Ia), (Ib), (Ic), (II), (IIa), (IIb), (III) or any sub-group or embodiment thereof as defined herein.
A compound of the formula (I), (Ia), (Ib), (Ic), (II), (IIa), (IIb), (III) or any sub-group or embodiment thereof as defined herein for use in the prophylaxis or treatment of a disease state or condition mediated by protein kinase A.
The use of a compound of formula (I), (Ia), (Ib), (Ic), (II), (IIa), (IIb), (III) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I), (Ia), (Ib), (Ic), (II), (IIa), (IIb), (III) or any sub-group or embodiment thereof as defined herein.
A method for treating a disease or condition comprising or arising from abnormal cell growth or abnormally arrested cell death in a mammal, the method comprising administering to the mammal a compound of the formula (I), (Ia), (Ib), (Ic), (II), (IIa), (IIb), (III) or any sub-group or embodiment thereof as defined herein in an amount effective to inhibit protein kinase A activity.
A method of inhibiting protein kinase A, which method comprises contacting the kinase with a kinase-inhibiting compound of the formula (I), (Ia), (Ib), (Ic), (II), (IIa), (IIb), (III) or any sub-group or embodiment thereof as defined herein.
A method of modulating a cellular process (for example cell division) by inhibiting the activity of a protein kinase A using a compound of the formula (I), (Ia), (Ib), (Ic), (II), (IIa), (IIb), (III) or any sub-group or embodiment thereof as defined herein.
The use of a compound of the formula (I), (Ia), (Ib), (Ic), (II), (IIa), (IIb), (III) or any sub-group or embodiment thereof as defined herein for the manufacture of a medicament for the prophylaxis or treatment of a disease state or condition arising from abnormal cell growth or abnormally arrested cell death.
A method for treating a disease or condition comprising or arising from abnormal cell growth or abnormally arrested cell death in a mammal, which method comprises administering to the mammal a compound of the formula (I), (Ia), (Ib), (Ic), (II), (IIa), (IIb), (III) or any sub-group or embodiment thereof as defined herein in an amount effective in inhibiting abnormal cell growth.
A method for alleviating or reducing the incidence of a disease or condition comprising or arising from abnormal cell growth or abnormally arrested cell death in a mammal, which method comprises administering to the mammal a compound of the formula (I), (Ia), (Ib), (Ic), (II), (IIa), (IIb), (III) or any sub-group or embodiment thereof as defined herein in an amount effective in inhibiting abnormal cell growth.
A pharmaceutical composition comprising a novel compound of the formula (I), (Ia), (Ib), (Ic), (II), (IIa), (IIb), (III) or any sub-group or embodiment thereof as defined herein and a pharmaceutically acceptable carrier.

A compound of the formula (I), (Ia), (Ib), (Ic), (II), (IIa), (IIb), (III) or any sub-group or embodiment thereof as defined herein for use in medicine.
The use of a compound of the formula (I), (Ia), (Ib), (Ic), (II), (IIa), (IIb), (III) or any sub-group or embodiment thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I), (Ia), (Ib), (Ic), (II), (IIa), (IIb), (III) or any sub-group or embodiment thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I), (Ia), (Ib), (Ic), (II), (IIa), (IIb), (III) or any sub-group or embodiment thereof as defined herein.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I), (Ia), (Ib), (Ic), (II), (IIa), (IIb), (III) or any sub-group or embodiment thereof as defined herein.
The use of a compound of the formula (I), (Ia), (Ib), (Ic), (II), (IIa), (IIb), (III) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I), (Ia), (Ib), (Ic), (II), (IIa), (IIb), (III) or any sub-group or embodiment thereof as defined herein.
The use of a compound of the formula (I), (Ia), (Ib), (Ic), (II), (IIa), (IIb), (III) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.

Where they do not already apply, any one or more of the following optional provisos may apply in any combination to any one or more of formulae (I), (Ia), (Ib), (Ic), (II), (IIa), (IIb), (III) or any sub-group or embodiment thereof as defined herein, and for any one or more of the aspects of the invention set out above and elsewhere herein.

(a-i) When J¹-J² is (R⁷)C═C(R⁶) and E is a monocyclic or bicyclic group linked through a nitrogen atom to the ring containing T, then A contains no oxo substituent.
(a-ii) E is other than an unsubstituted or substituted indole group;
(a-iii) when J¹-J² is N═CH, then E-A(R¹)—NR²R³ is other than a group —S—(CH₂)₃—CONH₂ or —S—(CH₂)₃—CN.
(a-iv) When J¹-J² is CH═N, then E-A(R¹)—NR²R³ is other than a group —NH—(CH₂)ₙ—N(CH₂CH₃)₂ where n is 2 or 3.
(a-v) When J¹-J² is N═CH, then E-A(R¹)—NR²R³ is other than a group —NH—(CH₂)₂—NH₂ or —NH—(CH₂)₂—N(CH₃)₂.
(b-i) E may be other than an unsubstituted or substituted indole group wherein A is attached to the benzene ring of the indole group.
(b-ii) When E is a monocyclic or bicyclic group linked through a nitrogen atom to the ring containing T, and one of R² and R³ together with the nitrogen atom to which they are attached and one or more atoms from A form a saturated monocyclic heterocyclic group optionally containing a second heteroatom ring member, then J¹-J² may be other than (R⁷)C═C(R⁶).
(b-iii) The moiety E-A(R¹)—NR²R³ may be other than an aminoalkylamino or alkylaminoalkylamino group.
(b-iv) When R¹ is hydrogen, E may be other than an acyclic group X-G.
(b-v) When E is piperidine or pyrrolidine, the moiety A(R¹)—NR²R³ may be other than pyrrolidinylethyl or pyrrolidinylmethyl.

### GENERAL PREFERENCES AND DEFINITIONS

The following general preferences and definitions shall apply to each of the moieties A, E, J¹, J², T and R¹ to R⁹ and any sub-definition, sub-group or embodiment thereof, unless the context indicates otherwise.

Any references to Formula (I) herein shall be taken also to refer to formulae (Ia), (Ib), (Ic), (II), (IIa), (IIb), (III) and any other sub-group of compounds within formula (I), or embodiment thereof, unless the context requires otherwise.

In this specification, references to “the bicyclic group”, when used in regard to the point of attachment of the group E shall, unless the context indicates otherwise, be taken to refer to the group:

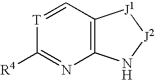

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic urea moieties (e.g. as in imidazolidin-2-one), cyclic thiourea moieties, cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

Examples of monocyclic non-aromatic heterocyclic groups include 5-, 6- and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine. In general, preferred non-aromatic heterocyclic groups include piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

One sub-group of substituents R¹⁰ is represented by R^{10a} which consists of substituents selected from halogen, hydroxy, trifluoromethyl, cyano, nitro, carboxy, amino, mono- or di-C₁₋₄ hydrocarbylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, OC(O)O, NR^{c}C(O)O, OC(O)NR^{c}, NR^{c}C(O)NR^{c}, S, SO, SO₂ NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, OC(O), NR^{c}C(O), OC(NR^{c}), C(O)O, C(O)NR^{c}, OC(O)O, NR^{c}C(O)O, OC(O)NR^{c} or NR^{c}C(O)NR^{c};

R^{c} is selected from hydrogen and C₁₋₄ hydrocarbyl.

Another sub-group of substituents R¹⁰ is represented by R^{10b} which consists of substituents selected from halogen, hydroxy, trifluoromethyl, cyano, amino, mono- or di-C₁₋₄ alkylamino, cyclopropylamino, carbocyclic and heterocyclic groups having from 3 to 7 ring members; a group R^{a}-Kb wherein R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, amino, mono- or di-C₁₋₄ alkylamino, carbocyclic and heterocyclic groups having from 3 to 7 ring members and wherein one or more carbon atoms of the C₁₋₈ hydrocarbyl group may optionally be replaced by O, S, SO, SO₂ or NR^{c}; provided that R^{a} is not a bond when R^{b} is hydrogen; and

R^{c} is selected from hydrogen and C₁₋₄ alkyl.

A further sub-group of substituents R¹⁰ is represented by R^{10c} which consists of substituents selected from:

halogen,
hydroxy,
trifluoromethyl,
cyano,
amino, mono- or di-C₁₋₄ alkylamino,
cyclopropylamino,
monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy;
a group R^{a}-R^{b};
R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂;
R^{b} is selected from hydrogen, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy;
and R^{b} is further selected from a C₁₋₈ hydrocarbyl group optionally substituted by one or more substituents selected from hydroxy, oxo, halogen, cyano, amino, mono- or di-C₁₋₄ alkylamino, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy, and wherein one or two carbon atoms of the C₁₋₈ hydrocarbyl group may optionally be replaced by O, S or NR^{c}; provided that R^{a} is not a bond when R^{b} is hydrogen; and
R^{c} is selected from hydrogen and C₁₋₄ alkyl.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

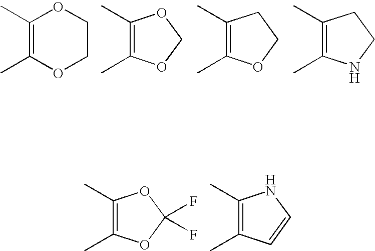

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) and sub-groups thereof as defined herein unless the context indicates otherwise. Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “saturated hydrocarbyl”, whether used alone or together with a suffix such as “oxy” (e.g. as in “hydrocarbyloxy”), refers to a non-aromatic hydrocarbon group containing no multiple bonds such as C═C and C—C.

Particular hydrocarbyl groups are saturated hydrocarbyl groups such as alkyl and cycloalkyl groups as defined herein.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by, for example, a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl, phenethyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### SPECIFIC EMBODIMENTS OF AND PREFERENCES FOR A, E, T, J¹, J² AND R¹ TO R¹⁰

In formula (I), T can be nitrogen or a group CR⁵ and J¹-J² can represent a group selected from N═C(R⁶), (R⁷)C═N, (R⁸)N—C(O), (R⁸)₂C—C(O) and (R⁷)C═C(R⁶). Thus the bicyclic group can take the form of, for example:
a purine (T is N, J¹-J² is N═C(R⁶));
a 3H-imidazo[4,5-b]pyridine (T is CR⁵, J¹-J² is N═C(R⁶));
a 7H-pyrrolo[2,3-d]pyrimidine (T is N, J¹-J² is (R⁷)C═C(R⁶));
a 1H-pyrrolo[2,3-b]pyridine (T is CR⁵, J¹-J² is (R⁷)C═C(R⁶));
a 5,7-dihydro-pyrrolo[2,3-d]pyrimidin-6-one (T is N, J¹-J² is (R⁸)₂C—C(O));
a 3H-[1,2,3]triazolo[4,5-d]pyrimidine (T is N, J¹-J² is N═N);
a 3H-[1,2,3]triazolo[4,5-b]pyridine (T is CR⁵, J¹-J² is N═N);
a 7,9-dihydro-purin-8-one (T is N, J¹-J² is (R⁸)N—C(O));
a 1H-pyrazolo[3,4-d]pyrimidine (T is N, J¹-J² is (R⁷)C═N); or
a pyrazolo[3,4-b]pyridine (T is CR⁵, J¹-J² is (R⁷)C═N).

R⁴ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. Typically, R⁴ is selected from hydrogen, halogen, C₁₋₅ saturated hydrocarbyl, cyano and CF₃. More typically, R⁴ is selected from hydrogen, chlorine, fluorine and methyl, and preferably R⁴ is hydrogen.

R⁵ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. Typically, R⁵ is selected from hydrogen, halogen, C₁₋₅ saturated hydrocarbyl, cyano and CF₃. Preferably, R⁵ is selected from hydrogen, chlorine, fluorine and methyl, and more preferably R⁵ is hydrogen.

R⁶ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. Typically, R⁶ is selected from hydrogen, halogen, C₁₋₅ saturated hydrocarbyl, cyano and CF₃. More typically R⁶ is selected from hydrogen, chlorine, fluorine and methyl, and preferably R⁶ is hydrogen.

R⁷ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. More typically R⁷ is selected from hydrogen, halogen, C₁₋₅ saturated hydrocarbyl, cyano and CF₃. Preferably, R⁷ is selected from hydrogen, chlorine, fluorine and methyl, and more preferably R⁷ is hydrogen.

R⁸ is selected from hydrogen, halogen, C₁₋₅ saturated hydrocarbyl, cyano, CONH₂, CONHR⁹, CF₃, NH₂, NHCOR⁹ and NHCONHR⁹. Typically, R⁶ is selected from hydrogen, halogen, C₁₋₅ saturated hydrocarbyl, cyano and CF₃. More typically, R⁸ is selected from hydrogen, chlorine, fluorine and methyl, and preferably R⁸ is hydrogen.

R⁹ is phenyl or benzyl each optionally substituted as defined herein. Particular groups R⁹ are phenyl and benzyl groups that are unsubstituted or are substituted with a solubilising group such as an alkyl or alkoxy group bearing an amino, substituted amino, carboxylic acid or sulphonic acid group. Particular examples of solubilising groups include amino-C₁₋₄-alkyl, mono-C₁₋₂-alkylamino-C₁₋₄-alkyl, di-C₁₋₂-alkylamino-C₁₋₄-alkyl, amino-C₁₋₄-alkoxy, mono-C₁₋₂-alkylamino-C₁₋₄-alkoxy, di-C₁₋₂-alkylamino-C₁₋₄-alkoxy, piperidinyl-C₁₋₄-alkyl, piperazinyl-C₁₋₄-alkyl, morpholinyl-C₁₋₄-alkyl, piperidinyl-C₁₋₄-alkoxy, piperazinyl-C₁₋₄-alkoxy and morpholinyl-C₁₋₄-alkoxy.

A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³. Within these constraints, the moieties E and R¹ can each be attached at any location on the group A.

The term “maximum chain length” as used herein refers to the number of atoms lying directly between the two moieties in question, and does not take into account any branching in the chain or any hydrogen atoms that may be present. For example, in the structure A shown below:

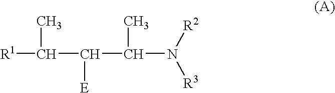

the chain length between R¹ and NR²R³ is 3 atoms whereas the chain length between E and NR²R³ is 2 atoms.

In general it is presently preferred that the linker group has a maximum chain length of 3 atoms (more preferably 1 or 2 atoms, and most preferably 2 atoms) extending between R¹ and NR²R³.

It is preferred that the linker group has a maximum chain length of 4 atoms, more typically 3 atoms, extending between E and NR²R³.

In one particularly preferred group of compounds, the linker group has a chain length of 1, 2 or 3 atoms extending between R¹ and NR²R³ and a chain length of 1, 2 or 3 atoms extending between E and NR²R³.

One of the carbon atoms in the linker group may optionally be replaced by an oxygen or nitrogen atom. When present, the oxygen or nitrogen atom preferably is linked directly to the group E.

When a nitrogen atom or oxygen atom are present, it is preferred that the nitrogen or oxygen atom and the NR²R³ group are spaced apart by at least two intervening carbon atoms.

In one particular group of compounds within formula (I), the linker atom linked directly to the group E is a carbon atom and the linker group A has an all-carbon skeleton.

The carbon atoms of the linker group A may optionally bear one or more substituents selected from oxo, fluorine and hydroxy, provided that the hydroxy group is not located at a carbon atom a with respect to the NR²R³ group, and provided also that the oxo group is located at a carbon atom a with respect to the NR²R³ group. Typically, the hydroxy group, if present, is located at a position P with respect to the NR²R³ group. In general, no more than one hydroxy group will be present. Where fluorine atoms are present, they may be present in a difluoromethylene or trifluoromethyl group, for example.

It will be appreciated that that when an oxo group is present at the carbon atom adjacent the NR²R³ group, the compound of the formula (I) will be an amide.

In one embodiment of the invention, no fluorine atoms are present in the linker group A.

In another embodiment of the invention, no hydroxy groups are present in the linker group A.

In a further embodiment, no oxo group is present in the linker group A.

In one group of compounds of the formula (I) neither hydroxy groups nor fluorine atoms are present in the linker group A, e.g. the linker group A is unsubstituted.

Preferably, when a carbon atom in the linker group A is replaced by a nitrogen atom, the group A bears no more than one hydroxy substituent and more preferably bears no hydroxy substituents.

In another group of compounds of the invention, the linker group A can have a branched configuration at the carbon atom attached to the NR²R³ group. For example, the carbon atom attached to the NR²R³ group can be attached to a pair of gem-dimethyl groups.

In one particular group of compounds of the formula (I), the portion R¹-A-NR²R³ of the compound is represented by the formula R¹-(G)ₖ-(CH₂)ₘ—X—(CH₂)ₙ—(CR⁶R⁷)ₚ—NR²R³ wherein G is NH, NMe or O; X is attached to the group E and is selected from (CH₂)ⱼ—CH, (CH₂)ⱼ—N, O—CH and (NH)ⱼ—CH; j is 0 or 1, k is 0 or 1, m is 0 or 1, n is 0, 1, 2, or 3 and p is 0 or 1, and the sum of j, k, m, n and p does not exceed 4; and R⁶ and R⁷ are the same or different and are selected from methyl and ethyl, or CR⁶R⁷ forms a cyclopropyl group.

One particular group CR⁶R⁷ is C(CH₃)₂.

Preferably X is (CH₂)ⱼ—CH.

Particular configurations are those wherein:
k is 0, m is 0 or 1, n is 0, 1, 2 or 3 and p is 0;
k is 0, m is 0 or 1, n is 0, 1 or 2 and p is 1;
X is (CH₂)ⱼ—CH, k is 1, m is 0, n is 0, 1, 2 or 3 and p is 0; and
X is (CH₂)ⱼ—CH, k is 1, m is 0, n is 0, 1 or 2 and p is 1.

In another embodiment, the portion R¹-A-NR²R³ of the compound is represented by the formula R¹—(CH₂)ₓ—X′—(CH₂)_{y}—NR²R³ wherein x is 0, 1 or 2, y is 0, 1 or 2 provided that the sum of x and y does not exceed 4; X′ is attached to the group E and is a group C(R^{x}) where (i) R^{x} is hydrogen or (ii) R^{x} together with R² constitutes an alkylene linking chain of up to 3 carbon atoms in length such that the moiety X′—(CH₂)_{y}—NR²R³ forms a 4 to 7 membered saturated heterocyclic ring.

In one group of compounds, R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group.

Within this group of compounds, R² and R³ may be independently selected from hydrogen, C₁₋₄ hydrocarbyl and C₁₋₄ acyl. Typically the hydrocarbyl group is an alkyl group, more usually a C₁, C₂ or C₃ alkyl group, for example a methyl group. In a particular sub-group of compounds, R² and R³ are independently selected from hydrogen and methyl and hence NR²R³ can be an amino, methylamino or dimethylamino group. In one embodiment, NR²R³ is an amino group. In another particular embodiment, NR²R³ is a methylamino group.

In another group of compounds, R² and R³ together with the nitrogen atom to which they are attached form a cyclic group selected from an imidazole group and a saturated monocyclic heterocyclic group having 4-7 ring members and optionally containing a second heteroatom ring member selected from O and N;

Within this group of compounds, is the sub-group wherein R² and R³ together with the nitrogen atom to which they are attached form a saturated monocyclic heterocyclic group having 4-7 ring members and optionally containing a second heteroatom ring member selected from O and N.

When NR²R³ forms a saturated monocyclic group, this may be substituted by one or more substituents independently selected from a group R¹⁰ as defined herein. More particularly the monocyclic heterocyclic group may be substituted by one or more C₁₋₄ alkyl groups. Alternatively, the monocyclic heterocyclic group may be unsubstituted.

The saturated monocyclic ring can be an azacycloalkyl group such as an azetidine, pyrrolidine, piperidine or azepane ring, and such rings are typically unsubstituted. Alternatively, the saturated monocyclic ring can contain an additional heteroatom selected from O and N, and examples of such groups include morpholine and piperazine. Where an additional N atom is present in the ring, this can form part of an NH group or an N—C₁₋₄alkyl group such as an N-methyl, N-ethyl, N-propyl or N-isopropyl group.

In a further group of compounds, one of R² and R³ together with the nitrogen atom to which they are attached and one or more atoms from the linker group A form a saturated monocyclic heterocyclic group having 4-7 ring members and optionally containing a second heteroatom ring member selected from O and N.

Examples of such compounds include compounds wherein NR²R³ and A form a unit of the formula:

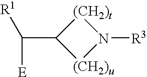

where t and u are each 0, 1, 2 or 3 provided that the sum of t and u falls within the range of 2 to 4.

Further examples of such compounds include compounds wherein NR²R³ and A form a group of the formula:

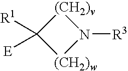

where v and w are each 0, 1, 2 or 3 provided that the sum of v and w falls within the range of 2 to 5. Particular examples of such compounds are those in which v and w are both 2.

Particular examples of the linker group A, together with their points of attachment to the groups R¹, E and NR²R³, are shown in Table 1 below.

TABLE 1

| | |
|---|---|
| | A1 |
| 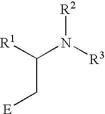 | |
| | A2 |
| 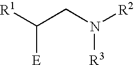 | |
| | A3 |
| 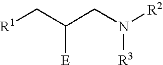 | |
| | A4 |
| 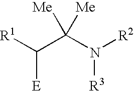 | |
| | A5 |
| 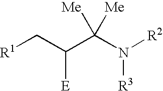 | |
| | A6 |
| 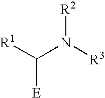 | |
| | A7 |
| 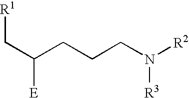 | |
| | A8 |
| 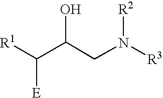 | |
| | A9 |
| 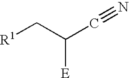 | |

| | |
|---|---|
| | A10 |
| 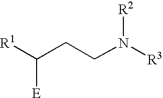 | |
| | A11 |
| 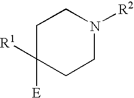 | |

Currently preferred groups include A1, A2, A3, A10 and A11. Particularly preferred groups include A1 and A11.

In formula (I), E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH.

When E is a monocyclic or bicyclic carbocyclic or heterocyclic group, it can be selected from the groups set out above in the section headed General Preferences and Definitions.

Particular cyclic groups E are monocyclic and bicyclic aryl and heteroaryl groups and, in particular, groups containing a six membered aromatic or heteroaromatic ring such as a phenyl, pyridine, pyrazine, pyridazine or pyrimidine ring, more particularly a phenyl, pyridine, pyrazine or pyrimidine ring, and more preferably a pyridine or phenyl ring.

Examples of bicyclic groups include benzo-fused and pyrido-fused groups wherein the group A and the pyrazole ring are both attached to the benzo- or pyrido-moiety.

In one embodiment, E is a monocyclic group.

Particular examples of monocyclic groups include monocyclic aryl and heteroaryl groups such as phenyl, thiophene, furan, pyrimidine, pyrazine and pyridine, phenyl being presently preferred.

Examples of non-aromatic monocyclic groups include cycloalkanes such as cylcohexane and cyclopentane, and nitrogen-containing rings such as piperidine, piperazine and piperazone.

One particular non-aromatic monocyclic group is a piperidine group and more particularly a piperidine group wherein the nitrogen atom of the piperidine ring is attached to the bicyclic group.

In one particular sub-group of compounds, E is selected from phenyl and piperidine groups.

It is preferred that the group A and the bicyclic group are attached to the group E in a meta or para relative orientation; i.e. A and the bicyclic group are not attached to adjacent ring members of the group E. Examples of groups such groups E include 1,4-phenylene, 1,3-phenylene, 2,5-pyridylene and 2,4-pyridylene, 1,4-piperidinyl, 1,4-piperindonyl, 1,4-piperazinyl, and 1,4-piperazonyl.

The groups E can be unsubstituted or can have up to 4 substituents R¹¹ which may be selected from the group R¹⁰ as hereinbefore defined. More typically however, the substituents R¹¹ are selected from hydroxy; CH₂CN, oxo (when E is non-aromatic); halogen (e.g. chlorine and bromine); trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

Typically, there are 0-3 substituents, more usually 0-2 substituents, for example 0 or 1 substituent. In one embodiment, the group E is unsubstituted.

The group E can be an aryl or heteroaryl group having five or six members and containing up to three heteroatoms selected from O, N and S, the group E being represented by the formula:

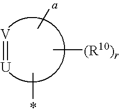

where * denotes the point of attachment to the bicyclic group, and “a” denotes the attachment of the group A;
r is 0, 1 or 2;
U is selected from N and CR^{12a}; and
V is selected from N and CR^{12b}; where R^{12a} and R^{12b} are the same or different and each is hydrogen or a substituent containing up to ten atoms selected from C, N, O, F, Cl and S provided that the total number of non-hydrogen atoms present in R^{12a} and R^{12b} together does not exceed ten;
or R^{12a} and R^{12b} together with the carbon atoms to which they are attached form an unsubstituted five or six membered saturated or unsaturated ring containing up to two heteroatoms selected from O and N; and
R¹⁰ is as hereinbefore defined.

In one particular group of compounds, E is a group:

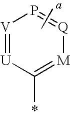

where * denotes the point of attachment to the pyrazole group, and “a” denotes the attachment of the group A;
P, Q and M are the same or different and are selected from N, CH and NCR¹⁰,
provided that the group A is attached to a carbon atom; and U, V and R¹⁰ are as hereinbefore defined.

Examples of R^{12a} and R^{12b} include hydrogen and substituent groups R¹⁰ as hereinbefore defined having no more than ten non-hydrogen atoms. Particular examples of R^{12a} and R^{12b} include methyl, ethyl, propyl, isopropyl, cyclopropyl, cyclobutyl, cyclopentyl, fluorine, chlorine, methoxy, trifluoromethyl, hydroxymethyl, hydroxyethyl, methoxymethyl, difluoromethoxy, trifluoromethoxy, 2,2,2-trifluoroethyl, cyano, amino, methylamino, dimethylamino, CONH₂, CO₂Et, CO₂H, acetamido, azetidinyl, pyrrolidino, piperidine, piperazino, morpholino, methylsulphonyl, aminosulphonyl, mesylamino and trifluoroacetamido.

When U is CR^{12a} and/or V is CR^{12b} the atoms or groups in R^{12a} and R^{12b} that are directly attached to the carbon atom ring members C are preferably selected from H, O (e.g. as in methoxy), NH (e.g. as in amino and methylamino) and CH₂ (e.g. as in methyl and ethyl).

In another particular group of compounds of the invention, E is a group:

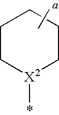

where X² is N or CH.

The group E can also be an acyclic group X-G wherein X is selected from CH₂, O, S and NH and G is a C₁₋₄ alkylene chain wherein one of the carbon atoms is optionally replaced by O, S or NH.

Examples of acyclic groups X-G include NHCH₂CH₂, NHCH₂CH₂CH₂, NHCH₂CH₂CH₂CH₂, OCH₂CH₂, OCH₂CH₂CH₂, OCH₂CH₂CH₂CH₂, SCH₂CH₂, SCH₂CH₂CH₂ and SCH₂CH₂CH₂CH₂. Particular acyclic groups X-G are NHCH₂CH₂ and NHCH₂CH₂CH₂.

Particular examples of the linker group E, together with their points of attachment to the group A (^{a}) and the bicyclic group (*) are shown in Table 2 below.

TABLE 2

| | |
|---|---|
| | B1 |
| 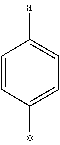 | |
| | B2 |
| 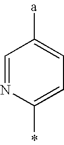 | |
| | B3 |
| 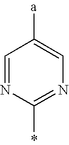 | |
| | B4 |
| 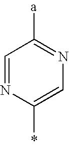 | |
| | B5 |
| 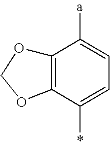 | |
| | B6 |
| 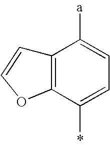 | |
| | B7 |
| 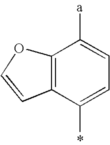 | |
| | B8 |
| 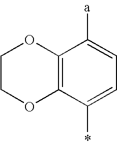 | |
| | B9 |
| 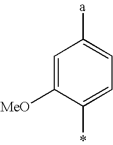 | |

| | |
|---|---|
| | B10 |
| 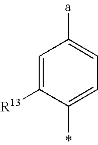 | |
| | B11 |
| 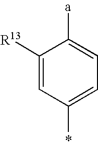 | |
| | B12 |
| 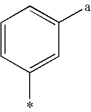 | |
| | B13 |
| 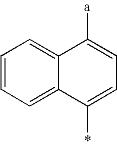 | |
| | B14 |
| 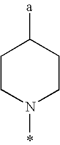 | |
| | B15 |
| 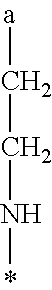 | |
| | B16 |
| 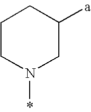 | |

In the table, the substituent group R¹³ is selected from methyl, chlorine, fluorine and trifluoromethyl.

The group R¹ is hydrogen or an aryl or heteroaryl group, wherein the aryl or heteroaryl group may be selected from the list of such groups set out in the section headed General Preferences and Definitions.

In one sub-group of compounds, R¹ is hydrogen.

In another sub-group of compounds, R¹ is an aryl or heteroaryl group.

When R¹ is aryl or heteroaryl, it can be monocyclic or bicyclic and, in one particular embodiment, is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

Examples of such groups include phenyl, naphthyl, thienyl, furan, pyrimidine and pyridine, with phenyl being presently preferred.

The aryl or heteroaryl group R¹ can be unsubstituted or substituted by up to 5 substituents, and examples of substituents are those listed in group R¹⁰ (or R^{10a}, R^{10b} or R^{10c}) above. Preferred substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In another embodiment, the group R¹ can have one or two substituents selected from fluorine, chlorine, trifluoromethyl, methyl and methoxy. When R¹ is a phenyl group, particular examples of substituent combinations include mono-chlorophenyl and dichlorophenyl.

When R¹ is a six membered aryl or heteroaryl group, a substituent may advantageously be present at the para position on the six-membered ring. Where a substituent is present at the para position, it is preferably larger in size than a fluorine atom.

In one embodiment, R¹ is selected from 4-fluorophenyl, 4-chlorophenyl and phenyl.

In formula (I), R⁴ is selected from hydrogen, halogen, C₁₋₅ saturated hydrocarbyl, cyano and CF₃. Preferred values for R⁴ include hydrogen and methyl.

In formula (I), R⁵ is selected from selected from hydrogen, halogen, C₁₋₅ saturated hydrocarbyl, cyano, CONH₂, CONHR⁹, CF₃, NH₂, NHCOR⁹ and NHCONHR⁹ where R⁹ is optionally substituted phenyl or benzyl.

More preferably, R⁵ is selected from selected from hydrogen, halogen, C₁₋₅ saturated hydrocarbyl, cyano, CF₃, NH₂, NHCOR⁹ and NHCONHR⁹ where R⁹ is optionally substituted phenyl or benzyl.

The group R⁹ is typically unsubstituted phenyl or benzyl, or phenyl or benzyl substituted by 1, 2 or 3 substituents selected from halogen; hydroxy; trifluoromethyl; cyano; carboxy; C₁₋₄alkoxycarbonyl; C₁₋₄ acyloxy; amino; mono- or di-C₁₋₄ alkylamino; C₁₋₄ alkyl optionally substituted by halogen, hydroxy or C₁₋₂ alkoxy; C₁₋₄ alkoxy optionally substituted by halogen, hydroxy or C₁₋₂ alkoxy; phenyl, five and six membered heteroaryl groups containing up to 3 heteroatoms selected from O, N and S; and saturated carbocyclic and heterocyclic groups containing up to 2 heteroatoms selected from O, S and N.

Particular examples of the moiety R⁵ include hydrogen, fluorine, chlorine, bromine, methyl, ethyl, hydroxyethyl, methoxymethyl, cyano, CF₃, NH₂, NHCOR^{9a} and NHCONHR^{9a} where R^{9a} is phenyl or benzyl optionally substituted by hydroxy, C₁₋₄ acyloxy, fluorine, chlorine, bromine, trifluoromethyl, cyano, C₁₋₄ hydrocarbyloxy (e.g. alkoxy) and C₁₋₄ hydrocarbyl (e.g. alkyl) optionally substituted by C₁₋₂ alkoxy or hydroxy.

### Particular and Preferred Sub-Groups of the Formula (I)

In one embodiment of the formula (I), the compounds can be represented by the general formula (II):

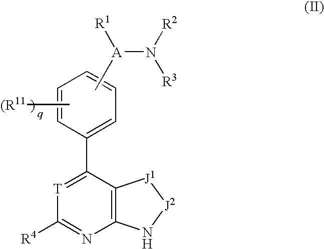

wherein the group A is attached to the meta or para position of the benzene ring, q is 0-4; T, J¹-J², A, R¹, R², R³ and R¹ are as defined herein in respect of formula (I) and sub-groups, examples and preferences thereof; and R¹¹ is a substituent group as hereinbefore defined. In formula (II), q is preferably 0, 1 or 2, more preferably 0 or 1 and most preferably 0.

Within formula (II), the portion R¹-A-NR²R³ of the compound can be represented by the formula R¹—(CH₂)ₓ—X′—(CH₂)_{y}—NR²R³ wherein x is 0, 1 or 2, y is 0, 1 or 2 provided that the sum of x and y does not exceed 4; X¹ is attached to the group E and is a group C(R^{x}) where (i) R^{x} is hydrogen or (ii) R^{x} together with R² constitutes an alkylene linking chain of up to 3 carbon atoms in length such that the moiety X′—(CH₂)_{y}—NR²R³ forms a 4 to 7 membered saturated heterocyclic ring.

For example, one sub-group of the compounds of the formula (II) can be represented by the formula (IIa):

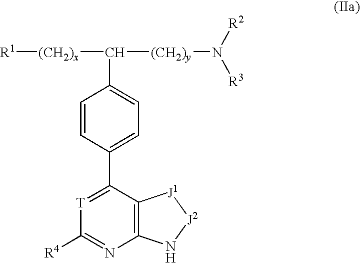

In formula (IIa), x is preferably 0 or 1 and y is 0, 1 or 2. In one embodiment, both x and y are 1. In another embodiment, x is 0 and y is 1.

Another sub-group of compounds within formula (II) can be represented by the formula (IIb):

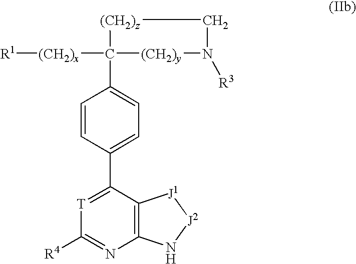

wherein R⁴, J¹-J², T, x and y are as hereinbefore defined and z is 0, 1 or 2 provided that the sum of y and z does not exceed 4. In one particular embodiment, y is 2 and z is 1.

In each of formulae (II), (IIa) and (IIb), and embodiments thereof, the group R¹ is preferably an optionally substituted aryl or heteroaryl group, and typically a monocyclic aryl or heteroaryl group of 5 or 6 ring members. Particular aryl and heteroaryl groups are phenyl, pyridyl, furanyl and thienyl groups, each optionally substituted as defined herein. Optionally substituted phenyl groups are particularly preferred.

Particular sub-groups of compounds in each of formulae (II), (IIa) and (IIb) consist of compounds in which R¹ is unsubstituted phenyl or, more preferably, phenyl bearing 1 to 3 (and more preferably 1 or 2) substituents selected from hydroxy; C₁₋₄ acyloxy; fluorine; chlorine; bromine; trifluoromethyl; cyano; C₁₋₄ hydrocarbyloxy and C₁₋₄ hydrocarbyl groups wherein the C₁₋₄ hydrocarbyloxy and C₁₋₄ hydrocarbyl groups are each optionally substituted by one or more C₁₋₂ alkoxy, halogen, hydroxy or optionally substituted phenyl or pyridyl groups; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; optionally substituted phenyl; optionally substituted pyridyl; and optionally substituted phenoxy; wherein the optional substituent for the phenyl, pyridyl and phenoxy groups are 1, 2 or 3 substituents selected from C₁₋₂ acyloxy, fluorine, chlorine, bromine, trifluoromethyl, cyano, and C₁₋₂ hydrocarbyloxy and C₁₋₂ hydrocarbyl groups wherein the C₁₋₂ hydrocarbyloxy and C₁₋₂ hydrocarbyl groups are each optionally substituted by methoxy or hydroxy.

More particular sub-groups of compounds within each of formulae (II), (IIa) and (IIb) consist of compounds wherein R¹ is unsubstituted phenyl or, more preferably, phenyl bearing 1 to 3 (and more preferably 1 or 2) substituents independently selected from hydroxy; C₁₋₄ acyloxy; fluorine; chlorine; bromine; trifluoromethyl; cyano; C₁₋₄ alkoxy or C₁₋₄ alkyl groups wherein the C₁₋₄ alkoxy and C₁₋₄ alkyl groups are each optionally substituted by one or more fluorine atoms or by C₁₋₂ alkoxy, hydroxy or optionally substituted phenyl; C₁₋₄ acylamino; benzoylamino; pyrrolidinocarbonyl; piperidinocarbonyl; morpholinocarbonyl; piperazinocarbonyl; optionally substituted phenyl; optionally substituted pyridyl; and optionally substituted phenoxy wherein the optionally substituted phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment within each of formulae (II), (IIa) and (IIb), R¹ is unsubstituted phenyl or a phenyl group substituted by 1 or 2 substituents independently selected from hydroxy; C₁₋₄ acyloxy; fluorine; chlorine; bromine; trifluoromethyl; trifluoromethoxy; difluoromethoxy; benzyloxy; cyano; C₁₋₄ hydrocarbyloxy and C₁₋₄ hydrocarbyl each optionally substituted by C₁₋₂ alkoxy or hydroxy.

More preferably, the group R¹ is a substituted phenyl group bearing 1 or 2 substituents independently selected from fluorine; chlorine; trifluoromethyl; trifluoromethoxy; difluoromethoxy; cyano; methoxy, ethoxy, i-propoxy, methyl, ethyl, propyl, isopropyl, tert-butyl and benzyloxy.

In one sub-group of compounds within each of formulae (II), (IIa) and (IIb), the group R¹ is a phenyl group having a substituent at the para position selected from fluorine, chlorine, trifluoromethyl, trifluoromethoxy, difluoromethoxy, benzyloxy, methyl, tert-butyl and methoxy, and optionally a second substituent at the ortho- or meta-position selected from fluorine, chlorine or methyl. Within this sub-group, the phenyl group can be monosubstituted. Alternatively, the phenyl group can be disubstituted.

In one embodiment within each of formulae (II), (IIa) and (IIb), R¹ is selected from 4-fluorophenyl, 4-chlorophenyl and phenyl.

In a particular sub-group of compounds within each of formulae (II), (IIa) and (IIb), the group R¹ is a monosubstituted phenyl group having a chlorine substituent at the para position.

In each of formulae (II), (IIa) and (IIb) and the above embodiments, sub-groups and examples thereof:
T is preferably N; and/or
R⁴ is hydrogen; and/or
J¹-J² represents a group selected from N═CH, HN—C(O), (Me)NC(O), (Et)NC(O) and HC═CH.

Another sub-group of compounds of the formula (I) has the general formula (III):

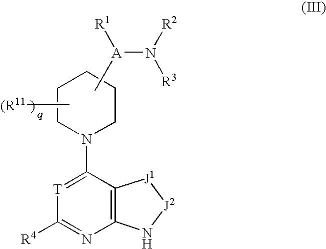

wherein the group A is attached to the 3-position or 4-position of the piperidine ring, q is 0-4; T, J¹-J², A, R¹, R², R³ and R⁴ are as defined herein in respect of formula (I) and sub-groups, examples and preferences thereof; and R¹¹ is a substituent group as hereinbefore defined. In formula (III), q is preferably 0, 1 or 2, more preferably 0 or 1 and most preferably 0.

The group R¹ is hydrogen or an aryl or heteroaryl group, wherein the aryl or heteroaryl group may be selected from the list of such groups set out in the section headed General Preferences and Definitions.

In one sub-group of compounds, R¹ is hydrogen.

In another sub-group of compounds, R¹ is an aryl or heteroaryl group.

When R¹ is aryl or heteroaryl, it can be monocyclic or bicyclic and, in one particular embodiment, is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

Examples of such groups include phenyl, naphthyl, thienyl, furan, pyrimidine and pyridine, with phenyl being presently preferred.

The aryl or heteroaryl group R¹ can be unsubstituted or substituted by up to 5 substituents, and examples of substituents are those listed in group R¹⁰ (or R^{10a} or R^{10b} or R^{10c}) above. Preferred substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In another embodiment, the group R¹ can have one or two substituents selected from fluorine, chlorine, trifluoromethyl, methyl and methoxy. When R¹ is a phenyl group, particular examples of substituent combinations include mono-chlorophenyl and dichlorophenyl.

When R¹ is a six membered aryl or heteroaryl group, a substituent may advantageously be present at the para position on the six-membered ring. Where a substituent is present at the para position, it is preferably larger in size than a fluorine atom.

In formula (I), R⁴ is selected from hydrogen, halogen, C₁₋₅ saturated hydrocarbyl, cyano and CF₃. Preferred values for R⁴ include hydrogen and methyl.

In formula (I), R⁵ is selected from selected from hydrogen, halogen, C₁₋₅ saturated hydrocarbyl, cyano, CONH₂, CONHR⁹, CF₃, NH₂, NHCOR⁹ and NHCONHR⁹ where R⁹ is optionally substituted phenyl or benzyl.

More preferably, R⁵ is selected from selected from hydrogen, halogen, C₁₋₅ saturated hydrocarbyl, cyano, CF₃, NH₂, NHCOR⁹ and NHCONHR⁹ where R⁹ is optionally substituted phenyl or benzyl.

The group R⁹ is typically unsubstituted phenyl or benzyl, or phenyl or benzyl substituted by 1, 2 or 3 substituents selected from halogen; hydroxy; trifluoromethyl; cyano; carboxy; C₁₋₄alkoxycarbonyl; C₁₋₄ acyloxy; amino; mono- or di-C₁₋₄ alkylamino; C₁₋₄ alkyl optionally substituted by halogen, hydroxy or C₁₋₂ alkoxy; C₁₋₄ alkoxy optionally substituted by halogen, hydroxy or C₁₋₂ alkoxy; phenyl, five and six membered heteroaryl groups containing up to 3 heteroatoms selected from O, N and S; and saturated carbocyclic and heterocyclic groups containing up to 2 heteroatoms selected from O, S and N.

Particular examples of the moiety R⁵ include hydrogen, fluorine, chlorine, bromine, methyl, ethyl, hydroxyethyl, methoxymethyl, cyano, CF₃, NH₂, NHCOR^{9a} and NHCONHR^{9a} where R^{9a} is phenyl or benzyl optionally substituted by hydroxy, C₁₋₄ acyloxy, fluorine, chlorine, bromine, trifluoromethyl, cyano, C₁₋₄ hydrocarbyloxy (e.g. alkoxy) and C₁₋₄ hydrocarbyl (e.g. alkyl) optionally substituted by C₁₋₂ alkoxy or hydroxy.

In another sub-group of compounds of the invention, A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from fluorine and hydroxy, provided that the hydroxy group when present is not located at a carbon atom α with respect to the NR²R³ group; and

R⁵ is selected from selected from hydrogen, C₁₋₅ saturated hydrocarbyl, cyano, CONH₂, CF₃, NH₂, NHCOR⁹ and NHCONHR⁹.

For the avoidance of doubt, it is to be understood that each general and specific preference, embodiment and example of the groups R¹ may be combined with each general and specific preference, embodiment and example of the groups R² and/or R³ and/or R⁴ and/or R⁵ and/or R⁹ and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below and include:
N-methyl-N′-(9H-purin-6-yl)-propane-1,3-diamine;
6-(3-methylamino-propylamino)-7,9-dihydro-purin-8-one;
1-(4-fluorophenyl)-N³-(9H-purin-6-yl)propane-1,3-diamine;
6-[3-amino-3-(4-fluorophenyl)propylamino]-7,9-dihydropurin-8-one;
1-(4-chlorophenyl)-N³-(9H-purin-6-yl)propane-1,3-diamine;
methyl-(4-(9H-purin-6-yl)benzyl)amine;
methyl-(3-(9H-purin-6-yl)benzyl)amine;
(4-(9H-purin-6-yl)phenyl)acetonitrile;
2-(4-(9H-purin-6-yl)phenyl)ethylamine;
2-(3-(9H-purin-6-yl)phenyl)ethylamine;
1-(9H-purin-6-yl)piperidine-4-carboxylic acid amide;
C-[1-(9H-purin-6-yl)piperidin-4-yl]methylamine;
6-[4-(aminophenylmethyl)piperidin-1-yl]-7,9-dihydropurin-8-one;
6-[4-(amino(4-chlorophenyl)methyl)piperidin-1-yl]-7,9-dihydropurin-8-one;
6-(4-aminomethylpiperidin-1-yl)-7,9-dihydropurin-8-one;
3-[3-(9H-purin-6-yl)-phenoxy]-propylamine;
C-[1-(1H-pyrazolo[3,4-d]pyrimidin-4-yl)-piperidin-4-yl]-methylamine;
C-[1-(7H-pyrrolo[2,3-d]pyrimidin-4-yl)-piperidin-4-yl]-methylamine;
C-phenyl-C-[4-(9H-purin-6-yl)-phenyl]-methylamine;
2-phenyl-1-[4-(9H-purin-6-yl)-phenyl]-ethylamine;
6-[4-(1-amino-2-phenylethyl)piperidin-1-yl]-7,9-dihydropurin-8-one;
6-(4-[4-(4-chlorophenyl)-piperidin-4-yl)-phenyl)-9H-purine;
4-{4-[4-(4-chloro-phenyl)-piperidin-4-yl]-phenyl}-7H-pyrrolo[2,3-d]pyrimidine;
C-phenyl-C-[1-(7H-pyrrolo[2,3-d]pyrimidin-4-yl)-piperidin-4-yl]-methylamine;
C-4-chlorophenyl-C-[1-(7H-pyrrolo[2,3-d]pyrimidin-4-yl)-piperidin-4-yl]-methylamine;
C-(4-chloro-phenyl)-C-[1-(9H-purin-6-yl)-piperidin-4-yl]-methylamine;
4-{4-[4-(4-chloro-phenyl)-piperidin-4-yl]-phenyl}-1H-pyrrolo[2,3-b]pyridine;
C-(4-chloro-phenyl)-C-[4-(9H-purin-6-yl)-phenyl]-methylamine;
C-(4-chlorophenyl)-C-[1-(1H-pyrrolo[2,3-b]pyridin-4-yl)piperidin-4-yl]methylamine;
{2-(4-chloro-phenyl)-2-[4-(1H-pyrrolo[2,3-b]pyridin-4-yl)-phenyl]-ethyl}-methyl-amine;
C-[1-(7H-pyrrolo[2,3-d]pyrimidin-4-yl)piperidin-3-yl]methylamine; and
C-(4-chlorophenyl)-C-[1-(1H-pyrrolo[2,3-b]pyridin-4-yl)piperidin-4-yl]methylamine;
   and salts, solvates, tautomers or N-oxides thereof.

### Salts, Solvates, Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (II) and (III) and sub-groups thereof unless the context indicates otherwise.

Salt forms may be selected and prepared according to methods described in Pharmaceutical Salts Properties, Selection, and Use, P. Heinrich Stahl (Editor), Camille G. Wermuth (Editor), ISBN: 3-90639-026-8, Hardcover, 388 pages, August 2002.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, when J¹-J² is N═CR⁶, the tautomeric forms A and B are possible for the bicyclic group.

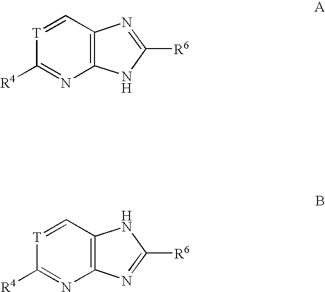

When J¹-J² is N═N, the tautomeric forms C and D are possible for the bicyclic group.

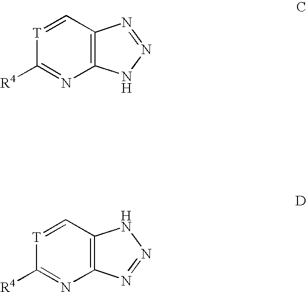

When J¹-J² is HN—CO, the tautomeric forms E, F and G are possible for the bicyclic group.

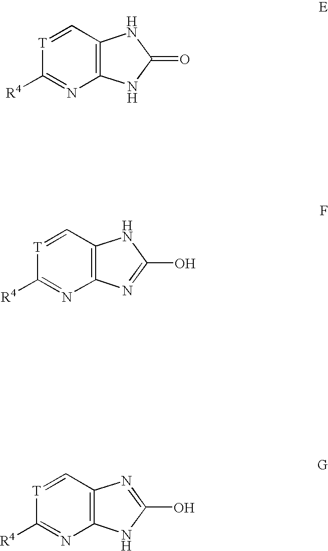

All such tautomers are embraced by formula (I).

Other examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

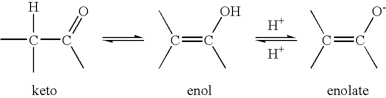

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl
(e.g., -Me, -Et, -nPr, -iPr, -nBu, -sBu, -iBu, -tBu);
C₁₋₇aminoalkyl
(e.g., aminoethyl; 2-(N,N-diethylamino)ethyl; 2-(4-morpholino)ethyl); and acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl; (4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

### Methods for the Preparation of Compounds of the Formula (I)

In this section, references to compounds of the formula (I) include formulae (II) and (III) and each of the sub-groups thereof as defined herein unless the context requires otherwise.

In a further aspect, the invention provides a process for the preparation of a compound of the formula (I) as defined herein.

Compounds of the formula (I) wherein E is an aryl or heteroaryl group can be prepared by reaction of a compound of the formula (X) with a compound of the formula (XI) where (X) and (XI) may be suitably protected and wherein A, E, and R¹ to R⁵ are as hereinbefore defined, one of the groups X and Y is chlorine, bromine or iodine or a trifluoromethanesulphonate (triflate) group, and the other one of the groups X and Y is a boronate residue, for example a boronate ester or boronic acid residue.

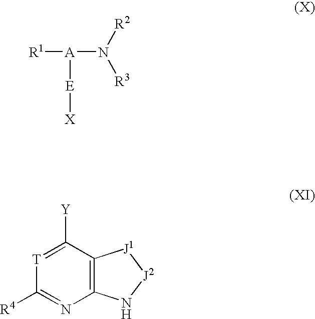

The reaction can be carried out under typical Suzuki Coupling conditions in the presence of a palladium catalyst such as tetrakis(triphenylphosphine)palladium or a palladacycle catalyst (e.g. the palladocycle catalyst described in R. B. Bedford & C. S. J. Cazin, Chem. Commun., 2001, 1540-1541) and a base (e.g. a carbonate such as potassium carbonate). The reaction may be carried out in a polar solvent, for example an aqueous solvent such as aqueous ethanol, or an ether such as dimethoxyethane or dioxane and the reaction mixture is typically subjected to heating, for example to a temperature of 80° C. or more, e.g. a temperature in excess of 100° C.

An illustrative synthetic route involving a Suzuki coupling step is shown in Scheme 1. In Scheme 1, the bromo compound (XII) in which E is an aryl or heteroaryl group, is converted to a boronic acid (XIII) by reaction with an alkyl lithium such as butyl lithium and a borate ester (iPrO)₃B. The reaction is typically carried out in a dry polar solvent such as tetrahydrofuran at a reduced temperature (for example −78° C.).

The resulting boronic acid (XIII) is then reacted with the N-protected chloro compound (XIV) in the presence of tetrakis(triphenylphosphine)palladium under the conditions described above. The protecting group PG (which can be for example a tetrahydropyranyl (THP) group) is then removed by treatment with an acid such as hydrochloric acid to give the compound of the formula (I).

In Scheme 1, where R² and/or R³ are hydrogen, the amino group NR²R³ is typically protecting with a suitable protecting group of which examples are set out below. One particular protecting group which may be used in the context of a Suzuki coupling is the tert-butoxycarbonyl group which can be introduced by reacting the amino group with di-tert-butylcarbonate in the presence of a base such as triethylamine. Removal of the protecting group is typically accomplished at the same time as removal of the protecting group PG on the bicyclic group.

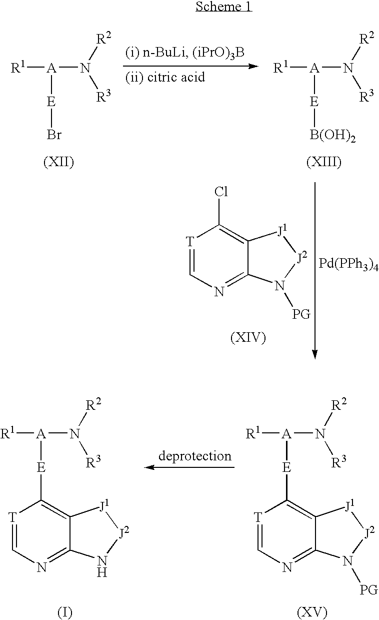

As an alternative to using a boronic acid (compound XIII) in the Suzuki coupling step, a boronate ester may be used instead. Boronate esters (for example a pinacolatoboronate) can be prepared from a compound of the formula (XII) by reaction with a diboronate ester such as bis(pinacolato)diboron in the presence of a phosphine such as tricyclohexyl-phosphine and a palladium (0) reagent such as tris(dibenzylideneacetone)-dipalladium (0). The formation of the boronate ester is typically carried out in a dry polar aprotic solvent such as dioxane with heating to a temperature of up to about 100° C., for example around 80° C.

Compounds of the formula (I) can also be prepared from the aldehyde compound (XVI) as shown in Scheme 2. The aldehyde compound (XVI) can be prepared by the reaction of the N-protected bicyclic chloro compound (XIV) with a boronic acid derivative of the formula (HO)₂B-E-CHO in the presence of a palladium catalyst Pd(PPh₃)₄ under the Suzuki coupling conditions described above. The aldehyde (XVI) can then be used to prepare a number of different compounds of the formula (I). Thus, for example, reaction of the aldehyde with tert-butyl sulphinamide in the presence of a suitable dehydrating agent, such as magnesium sulphate, and an acid catalyst, such as pyridinium p-toluenesulphonate, in dichloromethane at room temperature to give an intermediate tert-butyl sulphinylimine (not shown) followed by reaction with the Grignard reagent R¹—MgBr, where R¹ is an aryl or heteroaryl group (for example at room temperature or at reflux in tetrahydrofuran) gives the tert-butyl sulphinylamino derivative (XVII) which can then be hydrolysed and deprotected using hydrochloric acid in methanol to give the amine (XVIII).

The preparation of the corresponding compound (XIX) wherein A is CH and R¹ is hydrogen can be achieved by a reductive amination of the aldehyde (XVI) using an amine HNR²R³ and a reducing agent such as a borohydride (e.g. sodium borohydride) or a borohydride derivative (e.g. sodium cyanoborohydride or sodium triacetoxy borohydride) in a polar solvent such as ethanol or tetrahydrofuran (THF) usually at a reduced temperature.

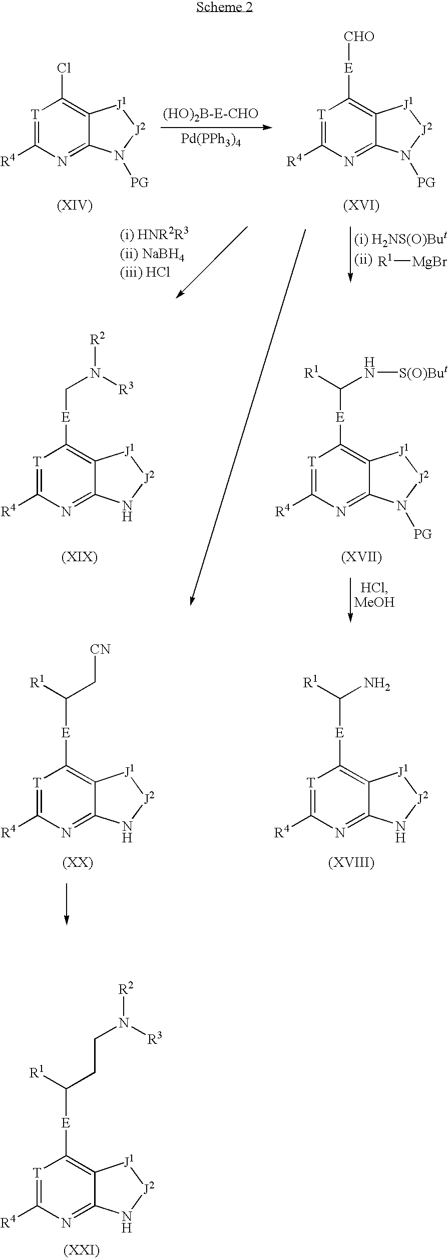

The formation of a compound of the formula (I) where ANR²R³ is CHCH₂CN or CHCH₂CH₂NR²R³ can be brought about by reacting the aldehyde (XVI) with malononitrile or ethylcyanoacetate in the presence of a base such as sodium or potassium hydroxide or an amine such a diethylamine or triethylamine under standard Knoevenagel condensation conditions (see Advanced Organic Chemistry by J. March, 4^{th} edition, John Wiley & Sons, 1992, pages 945-947 and references therein) to give an intermediate cyanoacrylate derivative (not shown). The cyanoacrylate derivative can then be reacted with a Grignard reagent R¹—MgBr and the product subjected to hydrolysis and decarboxylation to give a compound of the formula (XX) where R¹ is an aryl; or heteroaryl group. Alternatively, the cyanoacrylate derivative can be treated with a reducing agent that will selectively reduce the alkene double bond of the cyanoacrylate group without reducing the nitrile group to give the substituted acetonitrile derivative (XIV). A borohydride such as sodium borohydride may be used for this purpose The reduction reaction is typically carried out in a solvent such as ethanol and usually with heating, for example to a temperature up to about 65° C. The product is then subjected to hydrolysis and decarboxylation to give a compound of the formula (XX) where R¹ is hydrogen.

The substituted acetonitrile compound (XX) may then be reduced to the corresponding amine (XXI) by treatment with a suitable reducing agent such as Raney nickel and ammonia or hydrazine in ethanol.

Compounds of the formula (I) where A is CHCH₂ and R¹ is hydrogen may be prepared by condensing the aldehyde (XVI) with nitromethane in the presence of a base and then reducing the resulting nitroethene intermediate (not shown).

Compounds of the formula (I) wherein the group A contains a heteroatom which is attached directly to E, and E is an aryl or heteroaryl group can be formed by a process of the type illustrated in Scheme 3.

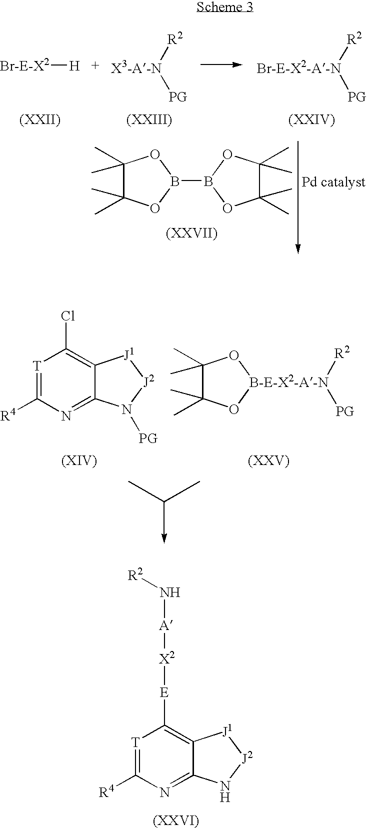

In Scheme 3, a bromoaryl or bromoheteroaryl derivative (XXII) where X² is 0 is reacted with a hydroxyalkyl compound (XXIII) where X³ is OH, A′ is the residue of the group A and PG is a protecting group such as a tert-butoxycarbonyl group, in a Mitsunobu coupling reaction. The Mitsunobu coupling reaction is typically carried out using diisopropylazodicarboxylate (DIAD) and triphenylphosphine as the coupling agent in a polar solvent such as THF.

Bromo compounds of the formula (XXIV) where X² is S or NH can also be formed by reacting a compound of the formula (XXII) where X² is S with a compound of the formula (XXIII) where X³ is a halogen, particularly bromine or chlorine. Compounds of the formula (XXIV) where X² is NH can be formed by the reductive amination of a compound of the formula (XXII) where X² is NH with a compound of the formula (XXIII) where X³ is an aldehyde group.

The resulting bromo compound (XXIV) is then reacted with the diboronate reagent (XXVII) in the presence of a palladium catalyst to give the boronate derivative (XXV) which can then be coupled with the chloro-bicyclic compound (XIV) under Suzuki conditions to give, after deprotection using an acid, a compound of the formula (XXVI).

In the preparative procedures outlined above, the coupling of the aryl or heteroaryl group E to the bicyclic group is accomplished by reacting a halo-purine (or deaza analogue thereof) or halo-aryl or heteroaryl compound with a boronate ester or boronic acid in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Compounds of the formula (I) in which the group A contains a nitrogen atom attached to the group E can be prepared by well known synthetic procedures from compounds of the formula (XXVIII) or a protected form thereof. Compounds of the formula (XXVIII) can be obtained by a Suzuki coupling reaction of a compound of the formula (XIV) (see Scheme 1) with a compound of the formula (HO)₂B-E-NH₂ or an N-protected derivative thereof.

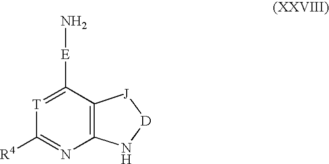

Compounds of the formula (I) wherein E is a non-aromatic cyclic group or an acyclic group and is linked to the bicyclic group by a nitrogen atom can be prepared by the reaction of a compound of the formula (XXIX) with an amine compound H₂N-G or a compound of the formula (XXX) or a protected derivative thereof, where G is as defined herein and the ring E represents a cyclic group E containing a nucleophilic NH group as a ring member.

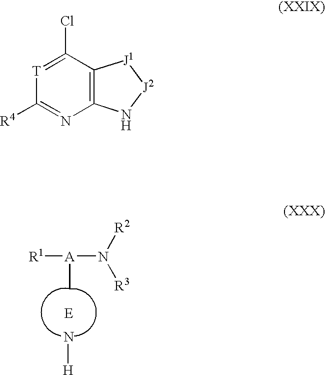

The reaction is typically carried out in a polar solvent such as an alcohol (e.g. ethanol, propanol or n-butanol) at an elevated temperature, for example a temperature in the region from 90° C. to 160° C. The reaction may be carried out in a sealed tube, particularly where the desired reaction temperature exceeds the boiling point of the solvent. When T is N, the reaction is typically carried out at a temperature in the range from about 100° C. to 130° C. but, when T is CH, higher temperatures may be required, for example up to about 160° C., and hence higher boiling solvents such as dimethylformamide may be used. In general, an excess of the nucleophilic amine will be used and/or an additional non-reacting base such as triethylamine will be included in the reaction mixture. Heating of the reaction mixture may be accomplished by normal means or by the use of a microwave heater.

In a variation on the above method, the compound of the formula (XXIX) may be reacted with a ketone of the formula (XXXI, A″ is a bond or an alkylene group such as methylene) as shown in Scheme 4.

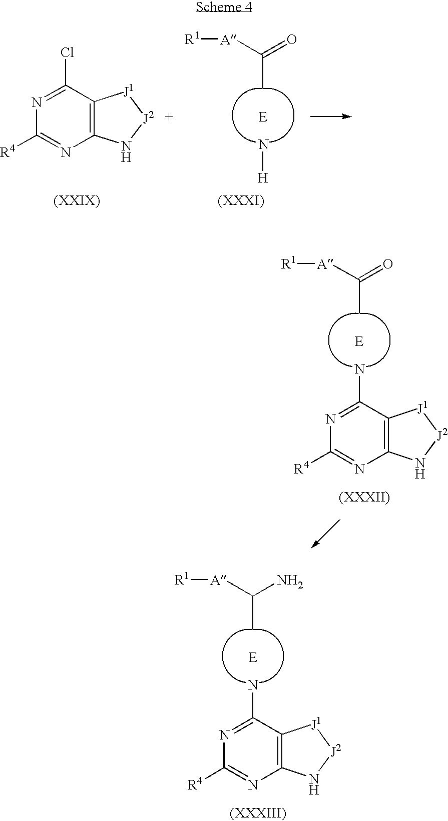

The reaction of the ketone (XXXI) with the chlorobicyclic compound (XXIX) is typically carried out in an alcoholic solvent such as n-butanol at an elevated temperature, for example in the region of 100° C. and in the presence of a non-interfering base such as triethylamine. The resulting ketone (XXXII) is then subjected to reductive amination using ammonium acetate in the presence of a reducing agent such as sodium cyanoborohydride in a polar solvent such as methanol.

Compounds of the formula (XXIX) are commercially available or can be prepared according to methods well known to the skilled person. For example, compounds of the formula (XXIX) where T is N and J¹-J² is CH═N can be prepared from the corresponding hydroxy compound by reaction with a chlorinating agent such as POCl₃. Compounds of the formula (XXIX) where J¹-J² is HN—C(O) can be prepared by the reaction of an ortho-diamino compound of the formula (XXXIV) with carbonyl di-imidazole in the presence of a non-interfering base such as triethylamine.

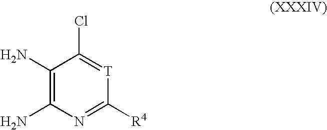

Compounds of the formula (XXIX) where T is CR⁵ and J¹-J² is (R⁷)H═CH(R⁶) can be prepared from the corresponding N-oxide of the formula (XXXV) by reaction with phosphorus oxychloride at an elevated temperature, for example the reflux temperature of POCl₃.

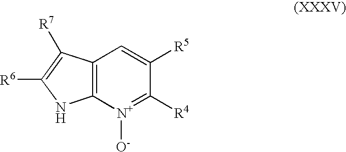

The starting materials of the formulae (X) and (XII) may be prepared by methods well known to the skilled person. For example, when E is an aryl or heteroaryl group, X is a halogen such as bromine, and the group R¹-A-NR²R³ is CH(CN)CH₂R¹, the compound of the formula (I) can be made according to the method illustrated in Scheme 5. The starting material for the synthetic route shown in Scheme 5 is the halo-substituted aryl- or heteroarylmethyl nitrile (XXXVI) in which X is a chlorine, bromine or iodine atom or a triflate group. The nitrile (XXXVI) is condensed with the aldehyde R¹CHO in the presence of an alkali such as sodium or potassium hydroxide in an aqueous solvent system such as aqueous ethanol. The reaction can be carried out at room temperature.

The resulting substituted acrylonitrile derivative (XXXVII) is then treated with a reducing agent that will selectively reduce the alkene double bond without reducing the nitrile group. A borohydride such as sodium borohydride may be used for this purpose to give the substituted acetonitrile derivative (XXXVIII). The reduction reaction is typically carried out in a solvent such as ethanol and usually with heating, for example to a temperature up to about 65° C. After reaction with a boronate compound of the formula (XI) where Y is a boronate ester or boronic acid residue under the Suzuki coupling conditions described above, the nitrile group can be reduced to the corresponding CH₂NH₂ group by treatment with a suitable reducing agent such as Raney nickel and ammonia in ethanol. Alternatively, the nitrile group can be reduced to the amino group and an amine-protecting group introduced before coupling with the boronate.

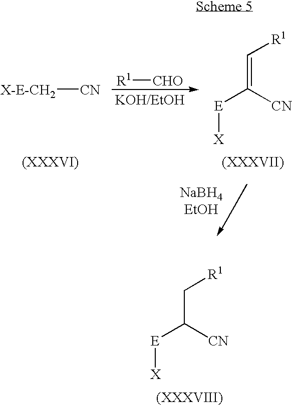

The synthetic route shown in Scheme 5 gives rise to amino compounds of the formula (X) and (XII) in which the aryl or heteroaryl group E is attached to the β-position of the group A relative to the amino group. In order to give amino compounds of the formula (X) or (XII) in which R¹ is attached to the β-position relative to the amino group, the functional groups on the two starting materials in the condensation step can be reversed so that a compound of the formula X-E-CHO wherein X is bromine, chlorine, iodine or a triflate group is condensed with a compound of the formula R¹—CH₂—CN to give a substituted acrylonitrile derivative which is then reduced to the corresponding acetonitrile derivative before coupling with the boronate (XI, Y=boronate residue) and reducing the cyano group to an amino group.

Compounds of the formula (X) or (XII) in which R¹ is attached to the α-position relative to the amino group can be prepared by the sequence of reactions shown in Scheme 6.

In Scheme 6, the starting material is a halo-substituted aryl- or heteroarylmethyl Grignard reagent (XXXIX), X=bromine or chlorine) which is reacted with the nitrile R¹—CN in a dry ether such as diethyl ether to give an intermediate imine (not shown) which is reduced to give the amine (XXXX) using a reducing agent such as lithium aluminium hydride. The amine (XXXX) can be reacted with the boronate ester or boronic acid (XI) under the Suzuki coupling conditions described above to yield a compound of the formula (I).

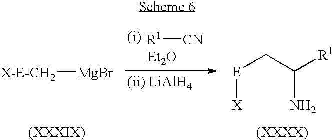

Compounds of the formula (X) and (XII) in which R¹ and E are connected to the same carbon atom can be prepared as shown in Scheme 7.

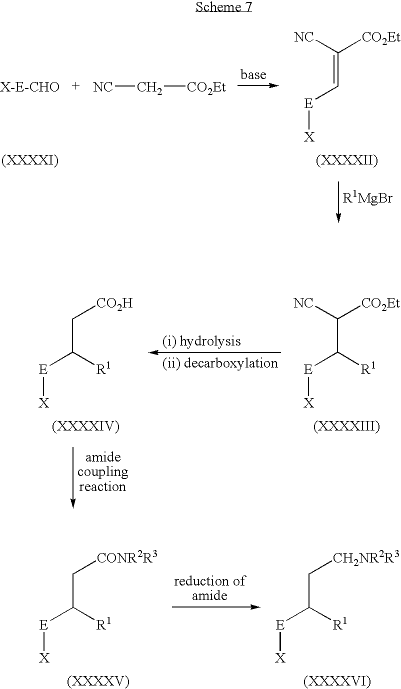

In Scheme 7, an aldehyde compound (XXXXI) where X is bromine, chlorine, iodine or a triflate group is condensed with ethyl cyanoacetate in the presence of a base to give a cyanoacrylate ester intermediate (XXXXII). The condensation is typically carried out in the presence of a base, preferably a non-hydroxide such as piperidine, by heating under Dean Stark conditions.

The cyanoacrylate intermediate (XXXXII) is then reacted with a Grignard reagent R¹MgBr suitable for introducing the group R¹ by Michael addition to the carbon-carbon double bond of the acrylate moiety. The Grignard reaction may be carried out in a polar non-protic solvent such as tetrahydrofuran at a low temperature, for example at around 0° C. The product of the Grignard reaction is the cyano propionic acid ester (XXXXIII) and this is subjected to hydrolysis and decarboxylation to give the propionic acid derivative (XXXXIV). The hydrolysis and decarboxylation steps can be effected by heating in an acidic medium, for example a mixture of sulphuric acid and acetic acid.

The propionic acid derivative (XXXXIV) is converted to the amide (XXXXV) by reaction with an amine HNR²R³ under conditions suitable for forming an amide bond. The coupling reaction between the propionic acid derivative (XXXXIV) and the amine HNR²R³ is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Where the amine HNR²R³ is ammonia, the amide coupling reaction can be carried out using 1,1′-carbonyldiimidazole (CDI) to activate the carboxylic acid before addition of the ammonia.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The amide (XXXXV) can be converted to a compound of the formula (I) wherein A has an oxo substituent next to the NR²R³ group by reaction with the boronate (XI) under the Suzuki coupling conditions as described above. The resulting amide of the formula (I) can subsequently be reduced using a hydride reducing agent such as lithium aluminium hydride in the presence of aluminium chloride to give a compound of the formula (I) in which NR²R³ is NH₂ and wherein A is CH—CH₂—CH₂—. The reduction reaction is typically carried out in an ether solvent, for example diethyl ether, with heating to the reflux temperature of the solvent.

Rather than reacting the amide (XXXXV) with the boronate or boronic acid (XI), the amide may instead be reduced with lithium aluminium hydride/aluminium chloride, for example in an ether solvent at ambient temperature, to give the corresponding amine (XXXXVI) which may be reacted with the boronate or boronic acid (XI) under the Suzuki coupling conditions described above to give the compound of the formula (I).

In order to obtain the homologue of the amine containing one fewer methylene group, the carboxylic acid (XXXXIV) can be converted to the azide by standard methods and subjected to a Curtius rearrangement (see Advanced Organic Chemistry, 4^{th} edition, by Jerry March, John Wiley & sons, 1992, pages 1091-1092.

Intermediate compounds of the formula (X) where the moiety X is a chlorine, bromine or iodine atom and A is a group CH—CH₂— can be prepared by the reductive amination of an aldehyde compound of the formula (XXXXVII):

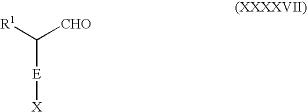

with an amine of the formula HNR²R³ under standard reductive amination conditions, for example in the presence of sodium cyanoborohydride in an alcohol solvent such as methanol or ethanol.

The aldehyde compound (XXXXVII) can be obtained by oxidation of the corresponding alcohol (XXXXVIII) using, for example, the Dess-Martin periodinane (see Dess, D. B.; Martin, J. C. J. Org. Soc., 1983, 48, 4155 and Organic Syntheses, Vol. 77, 141).

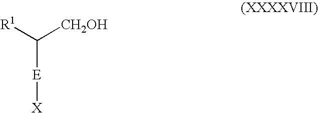

Compounds of the formula (I) where A, N and R² together form a spirocyclic group can be formed by the Suzuki coupling of a boronate or boronic acid compound of the formula (XI) with a spirocyclic intermediate of the formula (XXXXIX) or an N-protected derivative thereof.

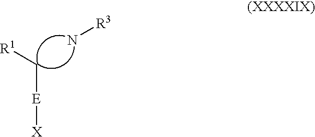

Spirocyclic intermediates of the formula (L) where R¹ is an aryl group such as an optionally substituted phenyl group, can be formed by Friedel Crafts alkylation of an aryl compound R¹—H with a compound of the formula (L):

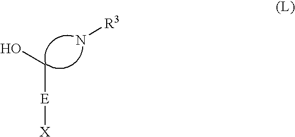

The alkylation is typically carried out in the presence of a Lewis acid such as aluminium chloride at a reduced temperature, for example less than 5° C.

In a further method for the preparation of a compound of the formula (I) wherein the moiety NR²R³ is attached to a CH₂ group of the moiety A, an aldehyde of the formula (LI) can be coupled with an amine of the formula HNR²R³ under reductive amination conditions as described above. In the formulae (LI) and (LII), A′ is the residue of the group A—i.e. the moieties A′ and CH₂ together form the group A. The aldehyde (LI) can be formed by oxidation of the corresponding alcohol (LII) using, for example, Dess-Martin periodinane.

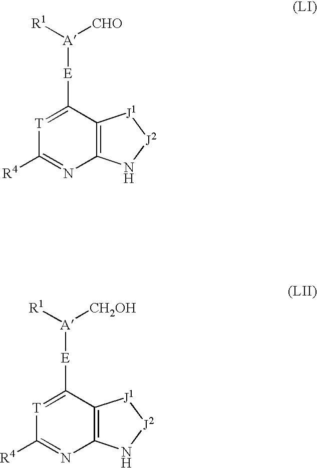

Once formed, many compounds of the formula (I) can be converted into other compounds of the formula (I) using standard functional group interconversions.

For example, compounds of the formula (I) or protected forms thereof wherein J¹-J² is CH═N can be converted into the corresponding compound where J¹-J² is N—C(CO) by bromination at the carbon atom in J¹-J² with a brominating agent such as N-bromosuccinimide (NBS) followed by hydrolysis with a mineral acid such as hydrochloric acid.

Other examples of interconversions include the reduction of compounds of the formula (I) in which the NR²R³ forms part of a nitrile group to the corresponding amine. Compounds in which NR²R³ is an NH₂ group can be converted to the corresponding alkylamine by reductive alkylation, or to a cyclic group.

Examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(—O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Isolation and Purification of the Compounds of the Invention

The compounds of the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

### Chemical Intermediates

Many of the chemical intermediates described above are novel and such novel intermediates form a further aspect of the invention.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g. lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams.

The active compound will be administered to a patient in need thereof (for example a human or animal patient) in an amount sufficient to achieve the desired therapeutic effect.

### Protein Kinase Inhibitory Activity

The activity of the compounds of the invention as inhibitors of protein kinase A and protein kinase B can be measured using the assays set forth in the examples below and the level of activity exhibited by a given compound can be defined in terms of the IC50 value. Preferred compounds of the present invention are compounds having an IC50 value of less than 1 μM, more preferably less than 0.1 μM, against protein kinase B.

Some of the compounds of the formula (I) are selective inhibitors of PKB relative to PKA, i.e. the IC₅₀ values against PKB are from 5 to 10 times lower, and more preferably greater than 10 times lower, than the IC₅₀ values against PKA.

### Therapeutic Uses

### Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lymphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB isoforms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

### Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

### Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Methods of Treatment

It is envisaged that the compounds of the formula (I) will useful in the prophylaxis or treatment of a range of disease states or conditions mediated by protein kinase A and/or protein kinase B. Examples of such disease states and conditions are set out above.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile manner.

A typical daily dose of the compound can be in the range from 100 picograms to 100 milligrams per kilogram of body weight, more typically 10 nanograms to 10 milligrams per kilogram of bodyweight although higher or lower doses may be administered where required. Ultimately, the quantity of compound administered will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topo II inhibitors
Alkylating Agents
Monoclonal Antibodies.

Anti-Hormones
Signal Transduction Inhibitors
Proteasome Inhibitors
DNA methyl transferases
Cytokines and retinoids
Radiotherapy.

For the case of protein kinase A inhibitors or protein kinase B inhibitors combined with other therapies the two or more treatments may be given in individually varying dose schedules and via different routes.

Where the compound of the formula (I) is administered in combination therapy with one or more other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through their common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, P13K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB. The term marker also includes markers which are characteristic of up regulation of PKA and/or PKB, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, or urine.

Identification of an individual carrying a mutation in PKA and/or PKB or a rearrangement of TCL-lor loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

### EXPERIMENTAL

The invention will now be illustrated, but not limited, by reference to the specific embodiments described in the following procedures and examples.

The starting materials for each of the procedures described below are commercially available, or are readily prepared from commercially available materials, unless otherwise specified.

Proton magnetic resonance (¹H NMR) spectra were recorded on a Bruker AV400 instrument operating at 400.13 MHz, in Me-d₃-OD at 27C, unless otherwise stated and are reported as follows: chemical shift δ/ppm (number of protons, multiplicity where s=singlet, d=doublet, t=triplet, q=quartet, m=multiplet, br-broad). The residual protic solvent MeOH (δ_{H}=3.31 ppm) was used as the internal reference.

In the examples, the compounds prepared were characterised by liquid chromatography and mass spectroscopy using the systems and operating conditions set out below. Where chlorine is present, the mass quoted for the compound is for ³⁵Cl. The operating conditions used are described below.

FractionLynx System

| | |
|---|---|
| System: | Waters FractionLynx (dual analytical/prep) |
| HPLC Pump: | Waters 2525 |
| Injector-Autosampler: | Waters 2767 |
| Mass Spec Detector: | Waters-Micromass ZQ |
| PDA Detector: | Waters 2996 PDA |

Acidic Analytical conditions:

| | |
|---|---|
| Eluent A: | H₂O (0.1% Formic Acid) |
| Eluent B: | CH₃CN (0.1% Formic Acid) |
| Gradient: | 5-95% eluent B over 5 minutes |
| Flow: | 2.0 ml/min |
| Column: | Phenomenex Synergi 4μ Max-RP 80 A, 50 × |
| | 4.6 mm |

MS conditions:

| | |
|---|---|
| Capillary voltage: | 3.5 kV |
| Cone voltage: | 25 V |
| Source Temperature: | 120° C. |
| Scan Range: | 125-800 amu |
| Ionisation Mode: | ElectroSpray Positive or ElectroSpray Positive & |
| | Negative |

Platform System

| | |
|---|---|
| HPLC System: | Waters 2795 |
| Mass Spec Detector: | Micromass Platform LC |
| PDA Detector: | Waters 2996 PDA |

Polar Analytical conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 1.5 ml/min |
| Column: | Phenomenex Synergi 4μ Hydro 80A, 50 × |
| | 4.6 mm |

MS conditions:

| | |
|---|---|
| Capillary voltage: | 3.5 kV |
| Cone voltage: | 30 V |
| Source Temperature: | 120° C. |
| Scan Range: | 165-700 amu |
| Ionisation Mode: | ElectroSpray Negative, Positive or Positive & |
| | Negative |

Acidic Analytical conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ Max-RP 80A, 50 × |
| | 2.0 mm |

LCT System 1

| | |
|---|---|
| HPLC System: | Waters Alliance 2795 Separations Module |
| Mass Spec Detector: | Waters/Micromass LCT |
| UV Detector: | Waters 2487 Dual λ Absorbance Detector |

Polar Analytical conditions:

| Eluent A: | Methanol | | |
|---|---|---|---|
| Eluent B: | 0.1% Formic Acid in Water | | |
| Gradient: | Time (mins) | A | B |
| | 0 | 10 | 90 |
| | 0.5 | 10 | 90 |
| | 6.5 | 90 | 10 |
| | 10 | 90 | 10 |
| | 10.5 | 10 | 90 |
| | 15 | 10 | 90 |

| | |
|---|---|
| Flow: | 1.0 ml/min |
| Column: | Supelco DISCOVERY C₁₈ 5 cm × |
| | 4.6 mm i.d., 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

LCT System 2

| | |
|---|---|
| HPLC System: | Waters Alliance 2795 Separations Module |
| Mass Spec Detector: | Waters/Micromass LCT |
| UV Detector: | Waters 2487 Dual λ Absorbance Detector |

Analytical conditions:

| Eluent A: | Methanol | | |
|---|---|---|---|
| Eluent B: | 0.1% Formic Acid in Water | | |
| Gradient: | Time (mins) | A | B |
| | 0 | 10 | 90 |
| | 0.6 | 10 | 90 |
| | 1.0 | 20 | 80 |
| | 7.5 | 90 | 10 |
| | 9 | 90 | 10 |
| | 9.5 | 10 | 90 |
| | 10 | 10 | 90 |

| | |
|---|---|
| Flow: | 1 ml/min |
| Column: | Supelco DISCOVERY C₁₈ 5 cm × |
| | 4.6 mm i.d., 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

Agilent System

| | |
|---|---|
| HPLC System: | Agilent 1100 series |
| Mass Spec Detector: | Agilent LC/MSD VL |
| Multi Wavelength | Agilent 1100 series MWD |
| Detector: | |
| Software: | HP Chemstation |

Chiral Analytical conditions:

| | |
|---|---|
| Eluent: | MeOH + 0.1% NH4/AcOH at room Temperature |
| Flow: | 1.0 ml/min |
| Total time: | 60.0 min |
| Inj. Volume: | 20 uL |
| Sample Conc: | 2 mg/ml |
| Column: | Astec, Chirobiotic V; 250 × 4.6 mm |

Chiral Preparative conditions 1:

| | |
|---|---|
| Eluent: | MeOH + 0.1% NH4/TFA at room Temperature |
| Flow: | 6.0 ml/min |
| Total time: | 50 min |
| Inj. Volume: | 50 uL |
| Sample Conc: | 20 mg/ml |
| Column: | Astec, Chirobiotic V; 250 × 10 mm |

In the examples below, the following key is used to identify the LCMS conditions used:

| | |
|---|---|
| PS-P | Platform System - polar analytical conditions |
| PS-A | Platform System - acid analytical conditions |
| FL-A | FractionLynx System - acidic analytical conditions |
| LCT1 | LCT System 1 - polar analytical conditions |
| LCT2 | LCT System 2 - polar analytical conditions |
| AS-CA | Agilent system - chiral analytical conditions |

### Example 1

### N-Methyl-N′-(9H-purin-6-yl)-propane-1,3-diamine

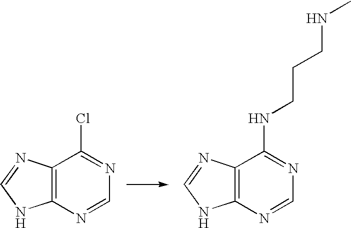

A solution of 6-chloropurine (0.3 g, 1.94 mmol) and N-methyl-1,3-propanediamine (0.61 ml, 5.82 mmol) in ethanol (5 ml) was heated at 120° C. (100 W) for 15 minutes in a sealed microwave tube with stirring in a CEM Discover microwave. Solvent was removed under reduce pressure and the residue was purified over flash silica chromatography eluting with methanol/dichloromethane (2:8) to yield the title compound as a white solid (0.197 g, 49% yield). LC/MS: (FL-A) Rₜ 0.36 [M+H]⁺ 207.22. ¹H NMR (DMSO) δ 1.92-2.03 (2H, m), 2.52 (2H, t), 2.81 (2H, t), 8.14 (1H, s), 8.20 (1H, s).

### Example 2

### 6-(3-Methylamino-propylamino)-7,9-dihydro-purin-8-one

### 2A. N-(8-Bromo-9H-purin-6-yl)-N′-methyl-propane-1,3-diamine

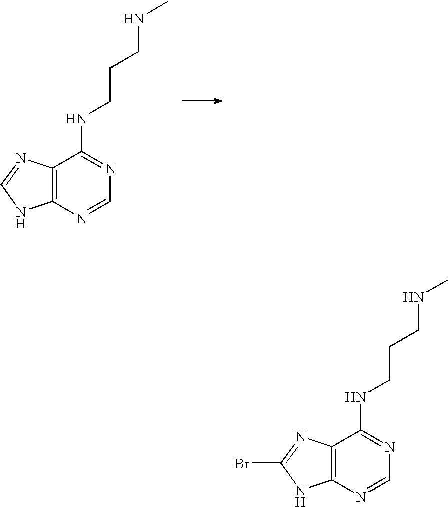

N-Bromosuccinimide (0.86 g, 4.84 mmol) was added to a solution of N-Methyl-N′-(9H-purin-6-yl)-propane-1,3-diamine (0.2 g, 0.97 mmol) in acetonitrile and the reaction mixture was stirred at room temperature for 64 hours. The solvent was removed under reduced pressure and the residue was purified over flash silica chromatography eluting with dichloromethane/methanol/acetic acid/water (90:18:3:2) to afford the title compound (0.044 g, 16% yield). LC/MS: (PS-P) Rₜ 1.72 [M+H]⁺ 284.93, 286.93.

### 2B. Methyl-[3-(8-oxo-8,9-dihydro-7H-purin-6-ylamino)-propyl]-carbamic Acid Tert-Butyl Ester

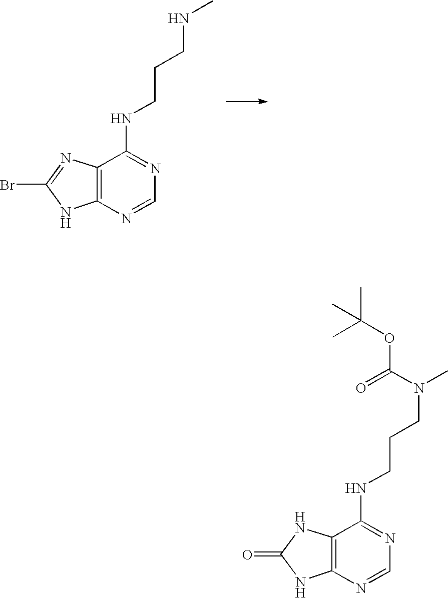

A solution of N-(8-bromo-9H-purin-6-yl)-N′-methyl-propane-1,3-diamine (0.04 g, 0.14 mmol) in concentrated hydrochloric acid (1 ml) was heated at 100° C. for 16 hours. The reaction mixture was transferred to iced water, neutralised with 2N sodium hydroxide, di-tert-butyl carbonate (0.03 g, 0.17 mmol) in tetrahydrofuran (1.5 ml) and sodium hydroxide (0.01 g, 0.14 mmol) were added. The reaction mixture was stirred for 1 hour, extracted with ethyl acetate. The organic layer was washed with brine, dried (MgSO₄) and solvent removed under reduced pressure. Purified over flash silica chromatography eluting with methanol/dichloromethane (5:95) to afford the title compound as a white solid (0.042 g, 93% yield). LC/MS: (PS-P) Rₜ 2.56 [M+H]⁺ 323.08.

### 2C. 6-(3-Methylamino-propylamino)-7,9-dihydro-purin-8-one

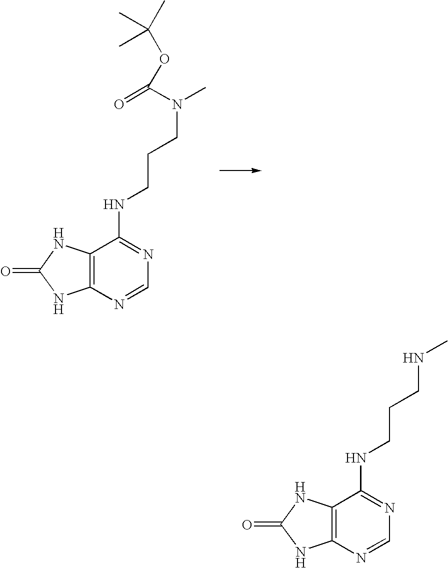

Methyl-[3-(8-oxo-8,9-dihydro-7H-purin-6-ylamino)-propyl]-carbamic acid tert-butyl ester (0.042 g, 0.13 mmol) was treated with 4M HCl in dioxane. The reaction mixture was stirred for 2 hours, solvent removed under reduced pressure to yield the title compound as a white solid (0.01 g, 35% yield). LC/MS: (PS-P) Rₜ 1.55 [M+H]⁺ 223.05. ¹H NMR (Me-d₃-OD) δ 2.04-2.13 (2H, m), 3.03 (2H, t), 3.45 (3H, s), 3.87 (2H, t), 8.32 (1H, s).

The following compounds were prepared in a similar manner:

### Example 3

### 1-(4-Fluorophenyl)-N³-(9H-purin-6-yl)propane-1,3-diamine

### 3A. [1-(4-Fluorophenyl)-3-(9H-purin-6-ylamino)propyl]carbamic Acid Tert-Butyl Ester

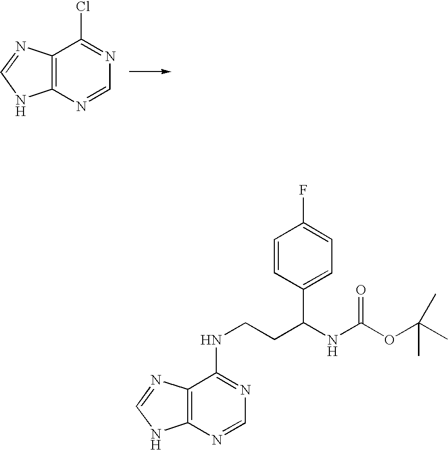

6-Chloropurine was reacted with [3-amino-1-(4-fluoro-phenyl)-propyl]-carbamic acid tert-butyl ester (Pharmacore, Inc, NC, USA) under the conditions described in Example 1A using a 2-fold excess of the amine and 5 equivalents of triethylamine to give the title compound: LC/MS: (LCT1) Rₜ 5.87 [M+H]⁺ 387.

### 3B. 1-(4-Fluorophenyl)-N³-(9H-purin-6-yl)propane-1,3-diamine

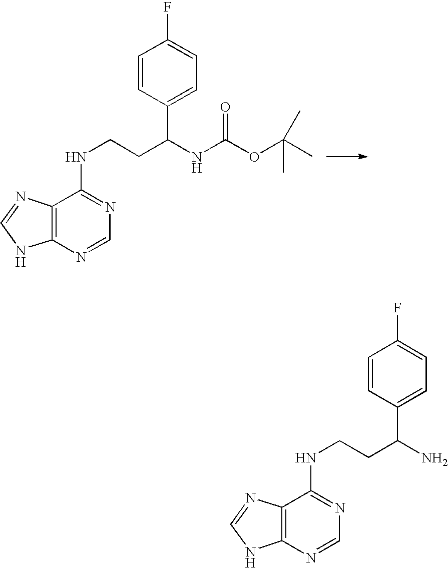

Removal of the Boc protecting group was accomplished using the method described in Example 2C to give the title compound: LC/MS: (LCT1) Rₜ 2.52 [M-NH₂]⁺ 270.

### Example 4

### 6-[3-Amino-3-(4-fluorophenyl)propylamino]-7,9-dihydropurin-8-one

### 4A. [3-(8-Bromo-9H-purin-6-ylamino)-1-(4-fluorophenyl)propyl]carbamic Acid Tert-Butyl Ester

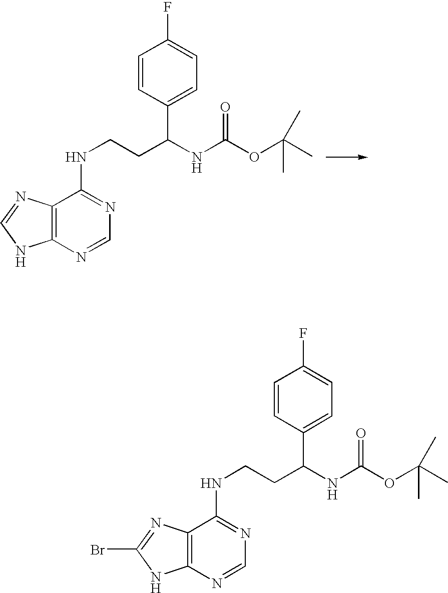

The product of Example 3A was brominated using N-bromosuccinimide according to the method of Example 2A to give the title compound: LC/MS: (LCT1) Rₜ 6.64 [M+H]⁺ 465.

### 4B. 6-[3-Amino-3-(4-fluorophenyl)propylamino]-7,9-dihydropurin-8-one

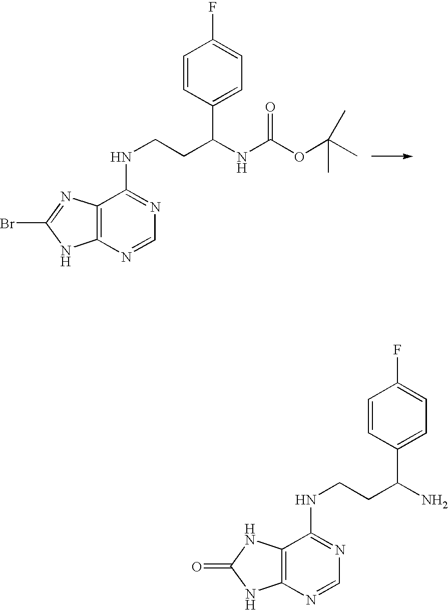

The bromo-compound of Example 4A was subjected to hydrolysis in hydrochloric acid using the method of Example 2B to give the title compound: LC/MS: (LCT1) Rₜ 3.05 [M-NH₂]⁺ 286.

### Example 5

### 1-(4-Chlorophenyl)-N³-(9H-purin-6-yl)propane-1,3-diamine

### 5A. [1-(4-Chlorophenyl)-3-(9H-purin-6-ylamino)propyl]carbamic Acid Tert-Butyl Ester

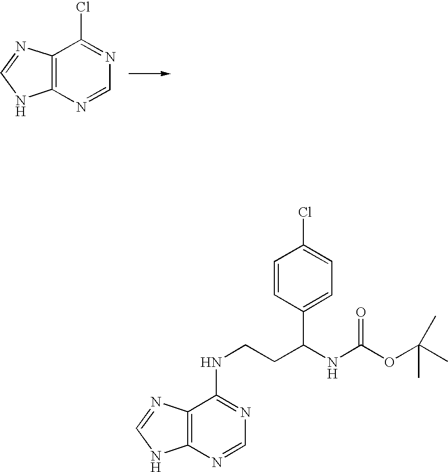

6-Chloropurine was reacted with [3-amino-1-(4-chloro-phenyl)-propyl]-carbamic acid tert-butyl ester (Pharmacore Inc, NC, USA) according to the method described in Example 1 to give the title compound: LC/MS: (LCT1) Rₜ 6.49 [M+H]⁺ 403.

### 5B. 1-(4-Chlorophenyl)-N³-(9H-purin-6-yl)propane-1,3-diamine

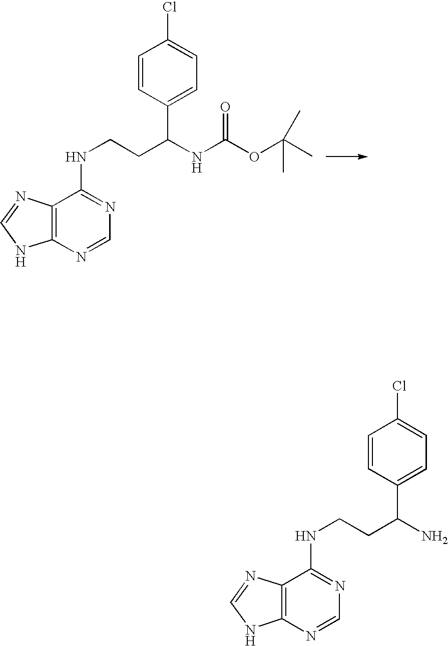

The product of Example 5A was deprotected by the method of Example 2C to give the title compound: LC/MS: (LCT1) Rₜ 3.02 [M-NH₂]⁺ 286.

### Example 6

### Methyl-(4-(9H-purin-6-yl)benzyl)amine

### 6A. 4-(9-(Tetrahydropyran-2-yl)-9H-purin-6-yl)benzaldehyde

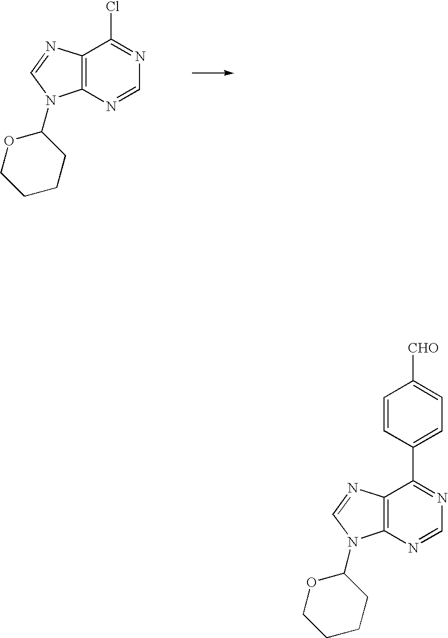

A mixture of 9-(tetrahydropyran-2-yl)-6-chloropurine (J. Am. Chem. Soc. 1961, 2574) (0.13 g, 0.55 mmol), 4-formylboronic acid (0.11 g, 0.75 mmol), 2M K₂CO₃ aq. (0.70 ml, 1.4 mmol) and Pd(PPh₃)₄ (0.03 g, 5 mol %) in 1,2-dimethoxy ethane (DME) (5 ml) was degassed and flushed with argon. The yellow solution was stirred at 85° C. under argon for 24 h, then cooled and filtered through Celite®, washing with EtOAc. The filtrate was concentrated and purified by flash column chromatography on silica gel, eluting with 50% EtOAc-hexanes, to give an off-white solid (0.354 g, 64%). LC/MS: (LCT1) Rₜ 6.15 [M+H-THP]⁺ 225

### 6B. Methyl-(4-(9-(tetrahydropyran-2-yl)-9H-purin-6-yl)benzyl)amine

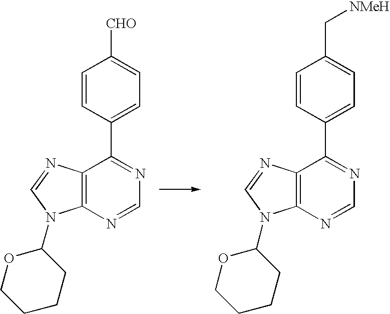

A solution of the aldehyde of Example 6A (0.25 g, 0.812 mmol) and methylamine (33% in EtOH, 25 ml) was stirred at room temperature for 2 hours, followed by evaporation of the solvent and excess amine. The white solid was redissolved in MeOH (25 ml) and NaBH₄ (0.05 g, 1.32 mmol) was added. After 30 minutes the solution was diluted with water (200 ml) and extracted with CH₂Cl₂ (100 ml). The extract was dried (Na₂SO₄), filtered and concentrated to give the amine as a colourless gum (0.231 g, 88%). LC/MS (LCT1): Rₜ 3.94 [M+H]⁺ 325.

### 6C. Methyl-(4-(9H-purin-6-yl)benzyl)amine

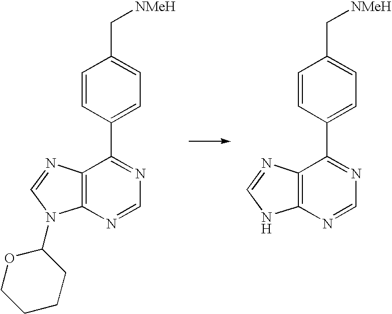

A solution of the amine of Example 6B in EtOH (15 ml) and 1M HCl (10 ml) was stirred at room temperature for 16 hours and was then evaporated to dryness. Solid phase extraction on SCX-II acidic resin, eluting with MeOH then 1M NH₃ in MeOH, gave the deprotected amine as a cream-coloured solid (0.142 g, 83%). LC/MS (LCT1): Rₜ 2.43 [M+H]⁺ 240.

### Example 7

### Methyl-(3-(9H-purin-6-yl)benzyl)amine

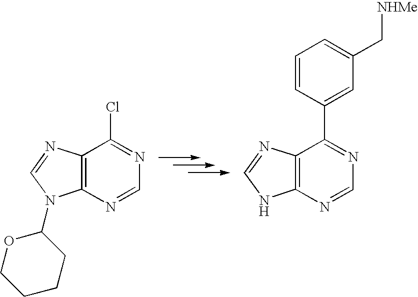

Starting from 6-chloro-9-(tetrahydro-pyran-2-yl)-9H-purine and 3-formylboronic acid and following the procedures set out in Example 6 gave the title compound: LC/MS (LCT1): Rₜ 2.77 [M+H]⁺ 240

### Example 8

### (4-(9H-purin-6-yl)phenyl)acetonitrile

### 8A. (4-(9-(Tetrahydropyran-2-yl)-9H-purin-6-yl)phenyl)acetonitrile

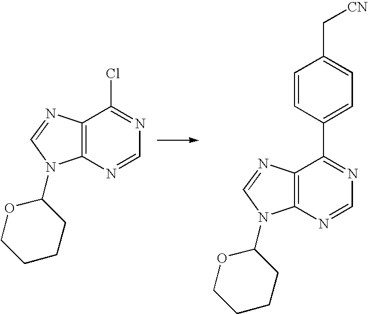

A solution of the N-protected chloropurine (0.27 g, 1.12 mmol), 4-cyanomethylphenylboronic acid (0.22 g, 1.37 mmol), 2M K₂CO₃ aq. (1.4 ml, 2.8 mmol) and Pd(PPh₃)₄ (0.03 g, 2.5 mol %) in DME (4 ml) was irradiated in a microwave reactor at 150° C. for 25 minutes. The organic layer was absorbed onto silica gel and purified by flash column chromatography, eluting 50% EtOAc-hexanes, to give a yellow solid (0.25 g, 70%). LC/MS (LCT1): Rₜ 5.84 [M+H-THP]⁺ 236.

### 8B. (4-(9H-purin-6-yl)phenyl)acetonitrile

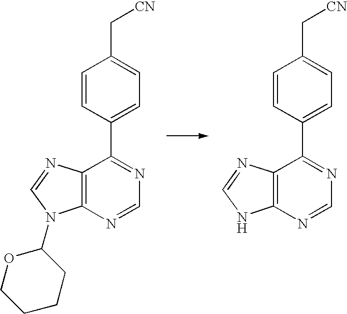

A mixture of the protected purine product of Example 8A (0.026 g, 0.081 mmol) and 1M HCl (1 ml) in EtOH (1.5 ml) was stirred at 80° C. for 6 hours and then evaporated to dryness. Filtration through SCX-II acidic resin, eluting with MeOH then 1M NH₃ in MeOH gave the title compound as a cream-coloured solid (0.015 g, 79%). LC/MS (LCT1): Rₜ 4.37 [M+H]⁺ 236.

### Example 9

### 2-(4-(9H-Purin-6-yl)phenyl)ethylamine

### 9A. 2-(4-(9-(Tetrahydropyran-2-yl)-9H-purin-6-yl)phenyl)ethylamine

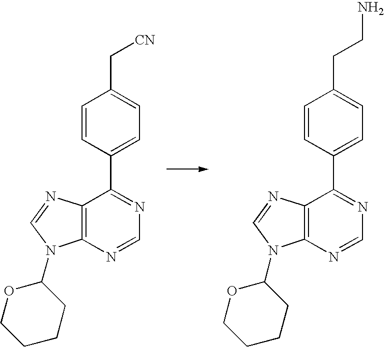

A suspension of Raney nickel in water (0.25 ml) was added to a solution of (4-(9-(tetrahydropyran-2-yl)-9H-purin-6-yl)phenyl)acetonitrile (0.021 g, (0.066 mmol) in 1,4-dioxane (2 ml). The suspension was stirred vigorously at 80° C. and hydrazine hydrate (0.5 ml) was added cautiously. After 30 minutes, the solution was cooled and filtered through SCX-II acidic resin, eluting with MeOH then 1M NH₃ in MeOH, to give a the title compound as a colourless oil (0.021 g, 98%) LC/MS (LCT1): Rₜ 4.22 [M+H-THP]⁺ 240.

### 9B. 2-(4-(9H-Purin-6-yl)phenyl)ethylamine

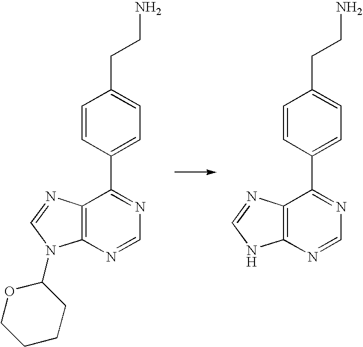

A solution of the protected purine of Example 9A (0.021 g, 0.065 mmol) and 1M HCl (2 ml) and EtOH (2 ml) was stirred at room temperature for 16 hours and then evaporated to dryness. Filtration through SCX-II acidic resin, eluting with MeOH then 1M NH₃ in MeOH, gave an off-white solid (0.011 g, 71%). LC/MS (LCT1): Rₜ 2.82 [M+H]⁺ 240.

The following compound was prepared by similar methods:

### Example 10

### 2-(3-(9H-purin-6-yl)phenyl)ethylamine

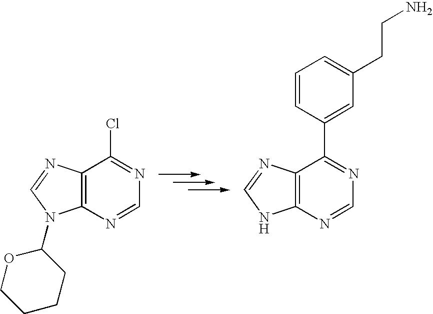

By reacting 6-chloro-9-(tetrahydro-pyran-2-yl)-9H-purine and 4-cyanomethylphenylboronic acid according to the method of Example 8A and then following the reduction and deprotection steps set out in Examples 9A and 9B, the title compound was prepared: LC/MS (LCT1): Rₜ 3.02 [M+H]⁺ 240.

### Example 11

### 1-(9H-Purin-6-yl)piperidine-4-carboxylic Acid Amide

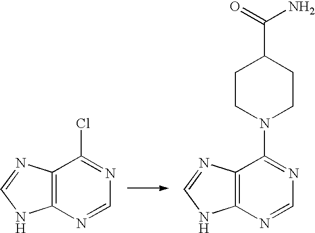

A solution of 6-chloropurine (0.500 g, 3.24 mmol), isonipecotamide (0.829 g, 6.47 mmol) and triethylamine (2.25 ml, 16.2 mmol) in n-butanol (32 ml) was stirred at 100° C. for 40 minutes. The suspension was concentrated and the residue was stirred with methanol (20 ml) for 1 hour. The insoluble white solid was collected and dried in vacuo to give the product (0.775 g, 96%). LC/MS: (LCT1) Rₜ 2.04 [M+H]⁺ 247.

### Example 12

### C-[1-(9H-Purin-6-yl)piperidin-4-yl]methylamine

### 12A. [1-(9H-Purin-6-yl)piperidin-4-ylmethyl]carbamic Acid Tert-Butyl Ester

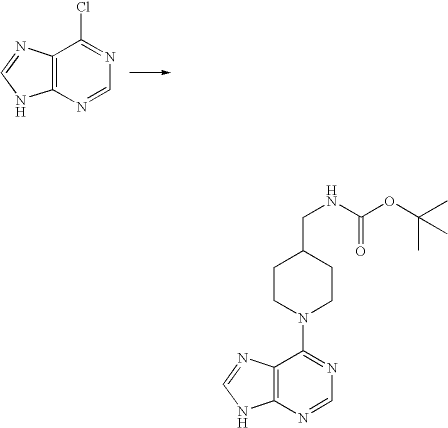

LC/MS: (LCT1) Rₜ 5.42 [M+H]⁺ 332.

### 12B. C-[1-(9H-Purin-6-yl)piperidin-4-yl]methylamine

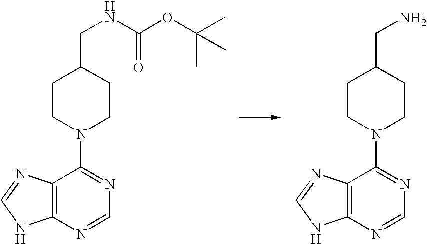

LC/MS (LCT1): Rₜ 1.18 [M+H]⁺ 233.

### Example 13

### 6-[4-(Aminophenylmethyl)piperidin-1-y]-7,9-dihydropurin-8-one

### 13A. 5,6-Diamino-4-chloropyrimidine

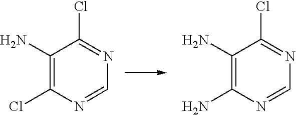

A mixture of 4,6-dichloro-5-aminopyrimidine (Aldrich Chemical Co.) (2.0 g, 12.2 mmol) and concentrated aqueous ammonia (20 ml) was heated to 100° C. in a sealed glass tube with vigorous stirring for 18 hours. The cooled tube was recharged with concentrated aqueous ammonia (8 ml), aggregates were broken up, and the mixture was reheated at 100° C. for a further 28 hours. The mixture was evaporated to dryness and the solids were washed with water (20 ml) and dried to give the product as yellow crystals (1.71 g, 97%). LC/MS (LCT1): Rₜ 1.59 [M+H]⁺ 147, 145.

### 13B. 6-Chloro-7,9-dihydropurin-8-one

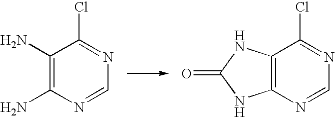

A mixture of the 5,6-diamino-4-chloropyrimidine of Example 13A (1.0 g, 6.92 mmol) and N,N′-carbonyldiimidazole (2.13 g, 13.2 mmol) in 1,4-dioxane (20 ml) was refluxed under argon for 48 hours. The solution was concentrated to a brown oil, which was triturated and washed with dichloromethane to give an off-white solid (1.02 g, 86%) LC/MS (LCT1): Rₜ 2.45 [M+H]⁺ 173, 171.

### 13C. 6-(4-Benzoylpiperidin-1-yl)-7,9-dihydropurin-8-one

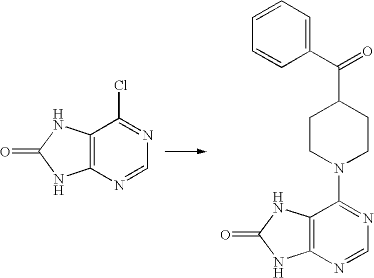

To a mixture of the 6-chloro-7,9-dihydropurin-8-one of Example 13B (0.100 g, 0.586 mmol) and (0.265 g, 1.172 mmol) in n-butanol (5.8 ml) was added triethylamine (0.408 ml, 2.930 mmol). After heating at 100° C. for 24 hours, solvent was removed and the resulting solid was triturated with methanol (10 ml). Filtration gave the title product as a white solid (0.121 g, 64%). LC/MS: (LCT1) Rₜ 5.70 [M+H]⁺ 324.

### 13D. 6-[4-(Aminophenylmethyl)piperidin-1-yl]-7,9-dihydropurin-8-one

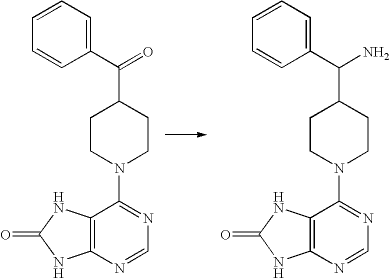

To a solution of the purinone of Example 13C (0.060 g, 0.186 mmol) in methanol (2 ml) was added ammonium acetate (172 mg, 2.227 mmol) and sodium cyanoborohydride (47 mg, 0.742 mmol). After refluxing for 2 days, the solution was cooled, then purified by solid phase extraction on SCX-II acidic resin, eluting with MeOH then 1M NH₃ in MeOH, which gave the title amine as a white solid (0.055 g, 92%). LC/MS (LCT1): Rₜ 3.90 [M+H]⁺ 325

### Example 14

### 6-[4-(Amino(4-chlorophenyl)methyl)piperidin-1-yl]-7,9-dihydropurin-8-one

### 14A. 6-(4-(4-Chlorobenzoyl)piperidin-1-yl)-7,9-dihydropurin-8-one

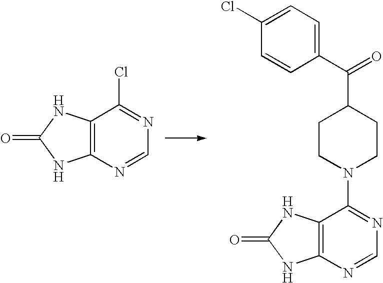

6-Chloro-7,9-dihydropurin-8-one was reacted with 4-(amino(4-chlorophenyl)methyl)piperidine by the method of Example 13C to give the title compound: LC/MS: (LCT1) Rₜ 6.42 [M+H]⁺ 358.

### 14B. 6-[4-(Amino(4-chlorophenyl)methyl)-piperidin-1-yl]-7,9-dihydropurin-8-one

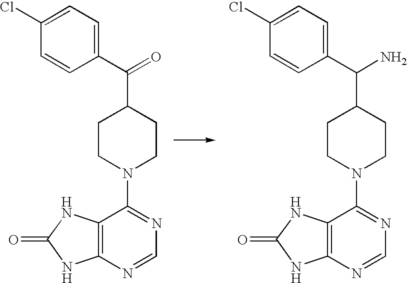

6-(4-(4-Chlorobenzoyl)piperidin-1-yl)-7,9-dihydropurin-8-one was subjected to the reductive amination method of Example 13D to give the title compound: LC/MS (LCT1): Rₜ 4.43 [M+H]⁺ 359.

### Example 15

### 6-(4-Aminomethylpiperidin-1-yl)-7,9-dihydropurin-8-one

### 15A. [1-(8-Oxo-8,9-dihydro-7H-purin-6-yl)piperidin-4-ylmethyl]carbamic Acid Tert-Butyl Ester

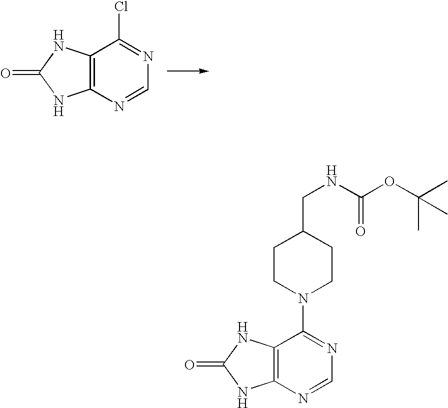

Following the method of Example 13C but using piperidin-4-ylmethylcarbamic acid tert-butyl ester as the amine yielded the title compound: LC/MS: (LCT1) Rₜ 5.70 [M+H]⁺ 349.

### 15B. 6-(4-Aminomethylpiperidin-1-yl)-7,9-dihydropurin-8-one

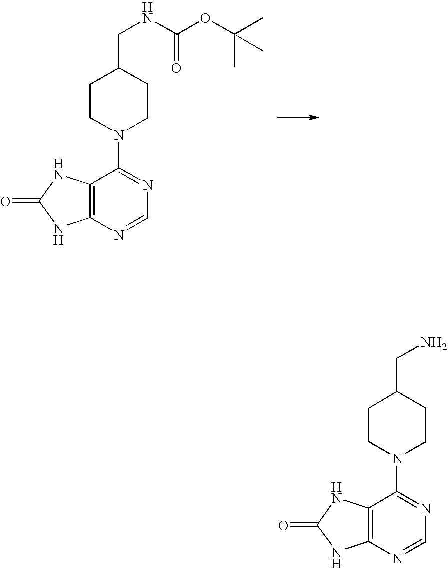

The product of Example 15A was deprotected by the method of Example 2C to give the title compound: LC/MS (LCT1): Rₜ 1.59 [M+H]⁺ 249.

### Example 16

### 3-[3-(9H-Purin-6-yl)-phenoxyl]-propylamine

### 16A. [3-(3-Bromo-phenoxy)-propyl]-carbamic Acid Tert-Butyl Ester

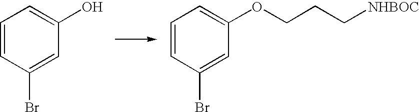

To a solution of 3-bromophenol (4.75 g, 27.2 mmol) in THF (40 ml) were added 3-hydroxypropylcarbamic acid tert-butyl ester (5.75 g, 32.8 mmol) in THF (30 ml) and triphenylphosphine (10.9 g, 41 mmol). The solution was cooled (ice bath) and diisopropylazodicarboxylate (DIAD) (7 ml, 35.5 mmol) was added dropwise. The solution was stirred at room temperature for 48 hours, and then hexane (100 ml) was added. The solution was washed with 1M NaOH solution (7×50 ml), then dried, concentrated and purified by flash column chromatography (silica gel, 4:1 hexane:ethyl acetate) to yield the product (3.28 g, 35%). ¹H NMR (250 MHz, d6-acetone) 1.42 (9H, s), 1.99 (2H, m), 3.28 (2H, q), 4.09 (2H, t), 6.10-6.20 (1H, br s), 6.95 (1H, m), 7.10-7.15 (2H, m), 7.25 (1H, t)

### 16B. {3-[3-(4,4,5,5-Tetramethyl-[1,3,2]dioxaborolan-2-yl)-phenoxy]-propyl}-carbamic Acid Tert-Butyl Ester

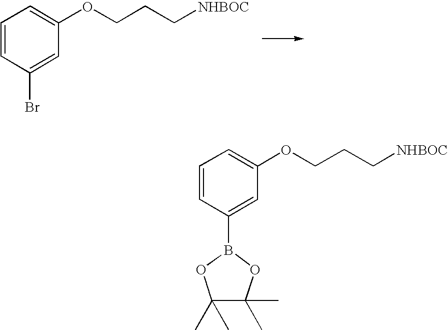

To tris(dibenzylideneacetone)dipalladium (0) (Pd₂ dba₃) (100 mg, 0.11 mmol) and tricyclohexylphosphine (76 mg, 0.27 mmol) was added dioxane (30 ml). The solution was degassed, and stirred at room temperature for 30 minutes. (Bis-pinacolato)diboron (1.44 g, 5.67 mmol), [3-(3-bromo-phenoxy)-propyl]-carbamic acid tert-butyl ester (1.80 g, 5.45 mmol) and potassium acetate (0.86 g, 8.76 mmol) were added, and the solution was heated at 80° C. for 16 h. After cooling to room temperature, the solution was poured into ethyl acetate (150 ml) and washed with water (50 ml) and brine (50 ml). The organic layer was dried, concentrated and purified by flash column chromatography (silica gel, 4:1 hexane:ethyl acetate) to yield the product (0.844 g, 43% yield). ¹H NMR (250 MHz, CDCl₃) 1.29 (9H, s), 1.37 (6H, s), 1.47 (6H, s), 1.99 (2H, ddt, J 6.2, 6.2, 6.2 Hz), 3.30-3.40 (2H, m), 3.98-4.07 (2H, m), 6.80-7.40 (4H, m)

### 16C. (3-{3-[9-(Tetrahydro-pyran-2-yl)-9H-purin-6-yl]-phenoxy}-propyl)-carbamic Acid Tert-Butyl Ester

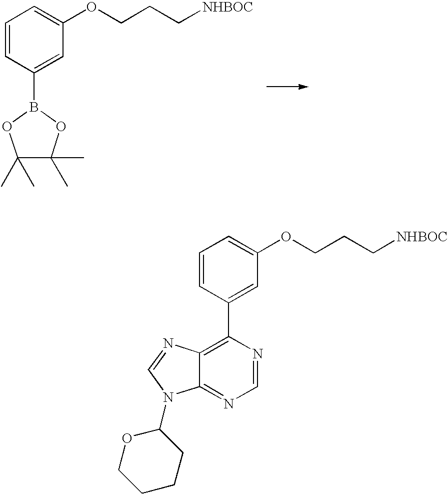

To a solution of {3-[3-(4,4,5,5-tetramethyl-[1,3,2]dioxaborolan-2-yl)-phenoxy]-propyl}-carbamic acid tert-butyl ester (0.252 mg, 0.68 mmol) in DME (7 ml) were added potassium carbonate (1 ml, 2M aqueous solution, 2 mmol), 6-chloro-9-(tetrahydro-pyran-2-yl)-9H-purine (157 mg, 0.65 mmol) and Pd(PPh₃)₄ (90 mg, 0.08 mmol). The solution was heated at reflux for 8 hours, then cooled to room temperature and poured into ethyl acetate (75 ml). The solution was washed with saturated NaHCO₃ (50 ml), brine (50 ml), then dried, concentrated and purified by flash column chromatography (SiO₂, 1:1 hexane:ethyl acetate) to yield the desired product. ¹H NMR (250 MHz, CDCl₃) 1.48 (9H, s), 1.60-2.30 (7H, m), 3.39 (2H, m), 3.84 (1H, dt, J 2.8, 11.0 Hz), 4.16-4.30 (3H, m), 5.00 (1H, br s), 5.88 (1H, dd, J 2.9, 9.8 Hz), 7.10 (1H, ddd, J 1.0, 2.6, 8.2 Hz), 7.48 (1H, m), 8.30-8.40 (2H, m), 8.45 (1H, m), 9.03 (1H, s)

### 16D. 3-[3-(9H-Purin-6-yl)-phenoxy]-propylamine

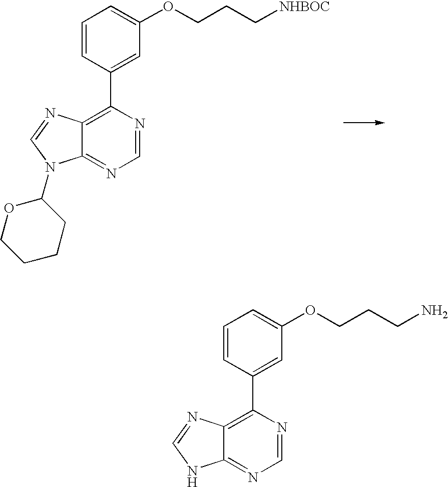

To a solution of (3-{3-[9-(tetrahydro-pyran-2-yl)-9H-purin-6-yl]-phenoxy}-propyl)-carbamic acid tert-butyl ester (77.5 mg, 0.17 mmol) in ethanol (1 ml) was added HCl (1 ml, 4M solution in dioxane, 4 mmol). The solution was stirred for 16 hours, and then concentrated under vacuum. The residue was dissolved in methanol and loaded onto an acidic resin SCX-2 cartridge, and washed with methanol (2×10 ml). Elution with 1M NH₃ in methanol gave the product (44 mg, 96% yield). LC/MS (LCT1) Rₜ 3.37 [M+H]⁺ 270

### Example 17

### C-[1-(1H-Pyrazolo[3,4-d]pyrimidin-4-yl)-piperidin-4-yl]-methylamine

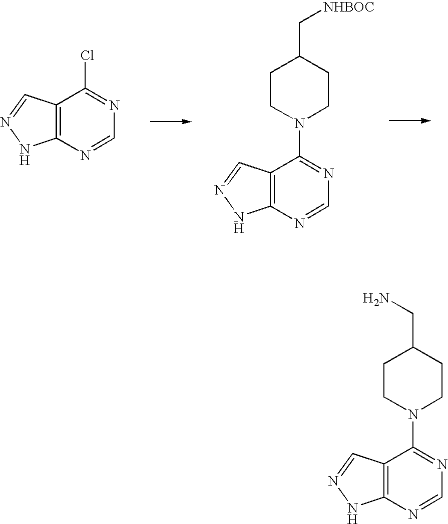

To a solution of 4-chloro-1H-pyrazolo[3,4-d]pyrimidine (J. Amer. Chem. Soc. 1957, 79, 6407-6413) (51 mg, 0.33 mmol) in ethanol (2 ml) was added triethylamine (100 μl, 0.72 mmol) and 4-(N-Boc-aminomethyl)piperidine (87 mg, 0.41 mmol). The solution was heated at 80° C. for 3 hours, and then cooled to room temperature. The solution was evaporated to dryness and the residue purified by recrystallisation (isopropanol) to yield the intermediate NH-BOC protected product (33 mg, 30% yield).

To the intermediate NH-BOC protected product (32 mg, 0.096 mmol) was added HCl (1 ml, 4M solution in dioxane, 4 mmol). The suspension was stirred at room temperature for 1 hour, and then diluted with diethyl ether (4 ml). The ethereal layer was discarded and the solid washed with a further portion of diethyl ether (2 ml). The ethereal layer was again discarded, and the resultant solid dried under high vacuum. The free base was liberated by dissolution of this material in methanol, loading onto an acidic resin SCX-2 cartridge, and elution from the cartridge with ammonia in methanol to give the title compound (21 mg, quantitative). LC/MS Rₜ 0.86 [M+H]⁺ 233

### Example 18

### C-[1-(7H-Pyrrolo[2,3-d]pyrimidin-4-yl)-piperidin-4-yl]-methylamine

### 18A. 6-Amino-5-(2,2-diethoxy-ethyl)-2-mercapto-pyrimidin-4-ol

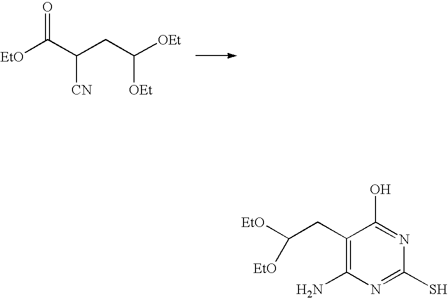

To ethanol (200 ml) was added sodium (2.05 g, 89 mmol) in small portions. The solution was stirred until complete dissolution of the sodium metal. 2-Cyano-4,4-diethoxy-butyric acid ethyl ester (J. Chem. Soc., 1960, 131-138) (9.292 g, 40.5 mmol) was then added as a solution in ethanol (50 ml), followed by addition of thiourea (3.08 g, 40.4 mmol). The solution was heated at 85° C. for 18 hours, and then cooled to room temperature. The solution was concentrated, and saturated aqueous ammonium chloride solution (150 ml) was added. The mixture was stirred at room temperature for 18 hours, after which time the solid was collected by filtration, and washed with water (20 ml) to yield the product (3.376 g, 36%).

### 18B. 6-Amino-5-(2,2-diethoxy-ethyl)-pyrimidin-4-ol

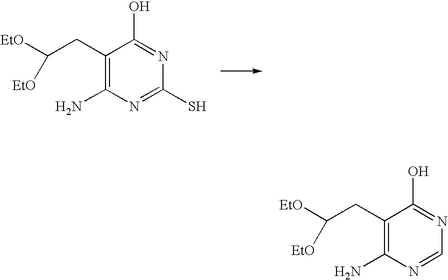

To a suspension of 6-amino-5-(2,2-diethoxy-ethyl)-2-mercapto-pyrimidin-4-ol (1.19 g, 4.6 mmol) in water (50 ml) was added Raney nickel (Aldrich Raney 2800 nickel, 4.8 ml). The mixture was heated at reflux for 1 hour, and then the hot solution was filtered through Celite®. The nickel residue was washed with further water (100 ml), and the washings were filtered through Celite. The aqueous filtrate was evaporated to dryness to yield the product (0.747 g, 71%).

### 18C. 7H-Pyrrolo[2,3-d]pyrimidin-4-ol

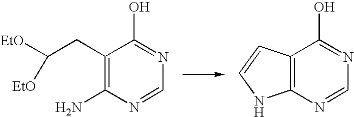

7H-Pyrrolo[2,3-d]pyrimidin-4-ol was prepared from 6-amino-5-(2,2-diethoxy-ethyl)-pyrimidin-4-ol by the method described in J. Chem. Soc., 1960, pp. 131-138.

### 18D. 4-Chloro-7H-pyrrolo[2,3-d]pyrimidine

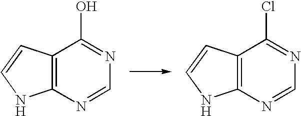

To 7H-pyrrolo[2,3-d]pyrimidin-4-ol (0.425 g, 3.14 mmol) was added phosphorus oxychloride (4 ml). The mixture was heated at reflux for 90 minutes and then cooled to room temperature. The solution was poured onto cracked ice, and extracted with chloroform (3×50 ml) and ethyl acetate (100 ml). The extracts were then dried and concentrated, and the residue obtained triturated with hot ethyl acetate (200 ml) to yield the desired product (0.204 g, 42%).

### 18E. [1-(7H-Pyrrolo[2,3-d]pyrimidin-4-yl)-piperidin-4-ylmethyl]-carbamic Acid Tert-Butyl Ester

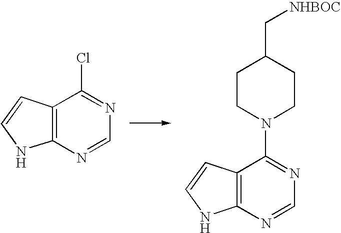

To a solution of 4-chloro-7H-pyrrolo[2,3-d]pyrimidine (67 mg, 0.44 mmol) in ethanol (1 ml) was added triethylamine (200 μl, 1.43 mmol) and 4-N-Boc-aminomethyl-piperidine (103 mg, 0.48 mmol). The solution was heated at 80° C. for 4 hours, and then cooled to room temperature. The precipitate was collected by filtration, recrystallised from ethanol-water (1:3) then dried under vacuum to yield the product (41 mg, 28%). LC/MS (LCT1) Rₜ 4.68 [M+H]⁺ 332

### 18F. C-[1-(7H-Pyrrolo[2,3-d]pyrimidin-4-yl)-piperidin-4-yl]-methylamine

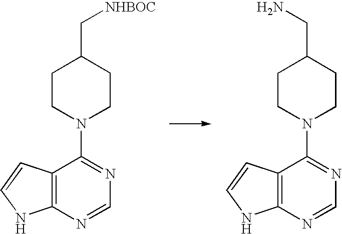

The product of Example 18E was deprotected by the method of Example 17 to give the title compound. LC/MS (LCT1) Rₜ 0.85 [M+H]⁺ 232

### Example 19

### C-Phenyl-C-[4-(9H-purin-6-yl)-phenyl]-methylamine

### 19A. 2-Methyl-propane-2-sulphinic acid 4-[9-(tetrahydro-pyran-2-yl)-9H-purin-6-yl]-benzylideneamide

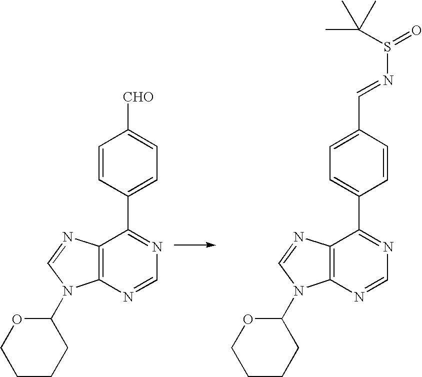

To a solution of racemic tert-butanesulphinamide (105 mg, 0.87 mmol) in dry dichloromethane (3.4 ml) was added pyridinium p-toluenesulphonate (6 mg, 0.025 mmol) and anhydrous magnesium sulphate (140 mg, 1.16 mmol) followed by the aldehyde of Example 6A (200 mg, 0.67 mmol). The mixture was stirred at room temperature under nitrogen for 48 hours (J. Am. Chem. Soc., 1997, 119, 9913). The reaction mixture was then filtered through a pad of Celite®, washed with dichloromethane and the solvent was evaporated in vacuo. The crude product was purified by flash silica column chromatography eluting with ethyl acetate/hexane (6:4) to afford the required compound as a white solid (124 mg, 0.30 mmol, 45%). LC/MS (LCT1) Rₜ 7.24 [M+H]⁺ 412.

### 19B. 2-Methyl-propane-2-sulphinic acid (phenyl-{4-[9-(tetrahydro-pyran-2-yl)-9H-purin-6-yl]-phenyl}-methyl)-amide

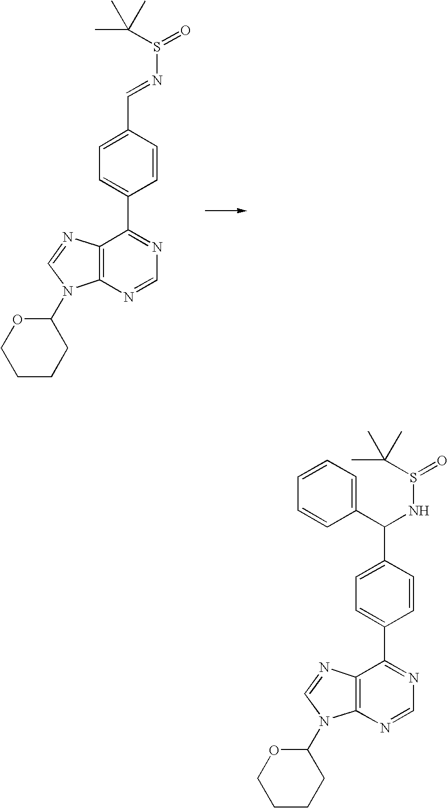

To a solution of the sulphinamide (37 mg, 0.09 mmol) in dry dichloromethane (1 ml) was added dropwise phenyl magnesium bromide 3M solution in diethyl ether (0.06 ml, 0.18 mmol), with stirring at −60° C. After stirring for 1 hour at −60° C. the temperature was increased slowly to 0° C. TLC analysis showed that the starting material had been consumed after 3 hours. The reaction mixture was quenched with saturated aqueous ammonium chloride (1 ml) and extracted with ethyl acetate. The combined organic layers were dried (MgSO₄) and concentrated in vacuo. The crude material was purified by flash silica column chromatography eluting with ethyl acetate/hexane (8:2) to afford the required compound (17 mg, 0.034 mmol, 38%). LC/MS (LCT1) Rₜ 7.14 [M+H]⁺ 490.

### 19C. C-Phenyl-C-[4-(9H-purin-6-yl)-phenyl]-methylamine

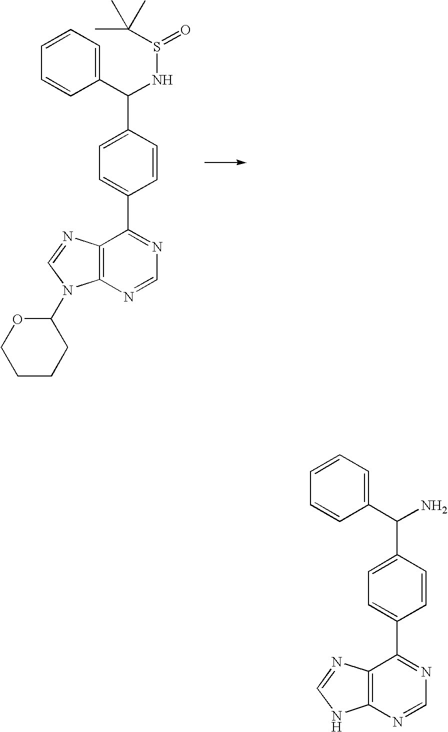

A solution of 2-methyl-propane-2-sulphinic acid (phenyl-{4-[9-(tetrahydro-pyran-2-yl)-9H-purin-6-yl]-phenyl}-methyl)-amide (16 mg, 0.033 mmol), ethanol (1.3 ml) and 1M aqueous HCl solution (1 ml) was stirred overnight at room temperature. The solvents were evaporated in vacuo and the crude material was passed through a basic resin NH₂ cartridge (2 g, 15 ml) eluting with methanol to afford the required compound (5.3 mg, 0.017 mmol, 53%). LC/MS (LCT1) Rₜ 4.19 [M+H]⁺ 302.

### Example 20

### 2-Phenyl-1-[4-(9H-purin-6-yl)-phenyl]-ethylamine

### 20A. 2-Methyl-propane-2-sulphinic acid (2-phenyl-1-{4-[9-(tetrahydro-pyran-2-yl-9H-purin-6-yl]-phenyl}-ethyl)-amide

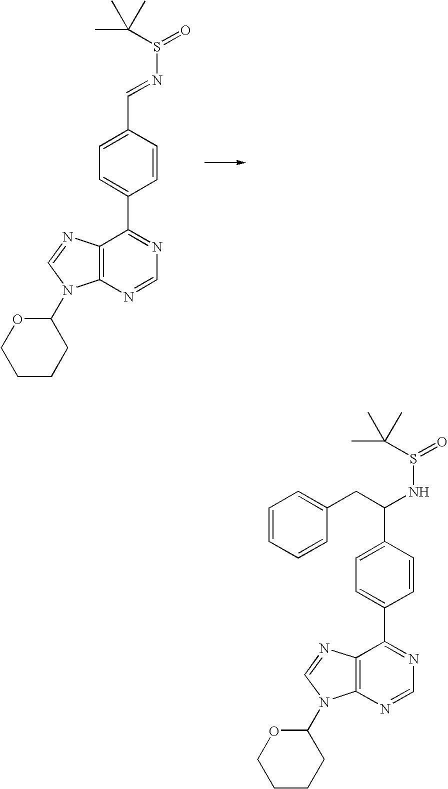

To a solution of the sulphinamide of Example 19A (38 mg, 0.09 mmol) in dry tetrahydrofuran (3 ml) was added dropwise benzyl magnesium chloride 2M solution in tetrahydrofuran (0.14 ml, 0.28 mmol), with stirring at room temperature. The solution was refluxed under nitrogen for 3 hours. The reaction mixture was cooled, quenched with saturated aqueous ammonium chloride (1 ml) and extracted with ethyl acetate. The combined organic layers were dried (MgSO₄) and concentrated in vacuo. The crude material was purified by flash silica column chromatography eluting with ethyl acetate/hexane (8:2) to afford the required compound (13 mg, 0.034 mmol, 29%). LC/MS (LCT1) Rₜ 7.34 [M+H]⁺ 504.

### 20B. 2-Phenyl-1-[4-(9H-purin-6-yl)-phenyl]-ethylamine

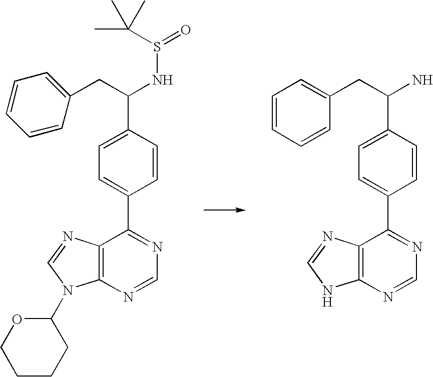

A solution of the product of Example 20A (2-methyl-propane-2-sulphinic acid (2-phenyl-1-{4-[9-(tetrahydro-pyran-2-yl)-9H-purin-6-yl]-phenyl}-ethyl)-amide) (13 mg, 0.026 mmol), methanol (0.5 ml) and HCl 4M solution in dioxane (0.04 ml) was stirred overnight at room temperature. The solvents were evaporated in vacuo and the crude material was passed through a basic resin NH₂ cartridge (2 g, 15 ml) eluting with methanol to afford the required compound (3.5 mg, 0.011 mmol, 43%). LC-MS (LCT1) Rₜ 4.37 [M+H]⁺ 316.

### Example 21

### 6-[4-(1-Amino-2-phenylethyl)piperidin-1-yl]-7,9-dihydropurin-8-one

### 21A. 4-(1-Hydroxy-2-phenylethyl)piperidine-1-carboxylic Acid Tert-Butyl Ester

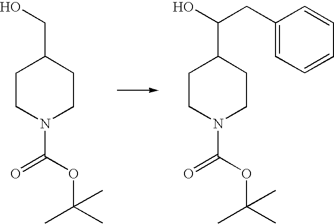

To a mixture of alcohol (0.503 g, 2.336 mmol), 4-methylmorpholine N-oxide (NMO) (356 mg, 3.037 mmol) and molecular sieves (4.0 g) in dichloromethane (23 ml) at 0° C. was added tetrapropylammonium perruthenate (TPAP) (41 mg, 0.117 mmol). After stirring for 2 hours at room temperature, the mixture was filtered through a pad of silica, washing with diethyl ether, and concentrated to give the crude aldehyde (not shown).

To a solution of the crude aldehyde in diethyl ether (20 ml) at 0° C. was added a solution of benzylmagnesium bromide (prepared from benzyl bromide (695 μl, 5.840 mmol) and magnesium (153 mg, 6.307 mmol) in diethyl ether (12 ml)). After stirring at room temperature for 15 hours, saturated aqueous ammonium chloride (150 ml) was added, the phases were separated and the aqueous phase extracted with diethyl ether (50 ml). The organic phases were combined, dried (magnesium sulphate) and concentrated, and the resulting crude product was purified by silica column chromatography (60% diethyl ether/hexane) to give the title alcohol as a clear oil (256 mg, 36%). LC/MS: (LCT1) Rₜ 7.11 [M+Na]⁺ 328.

### 21B. 4-Phenylacetylpiperidine-1-carboxylic Acid Tert-Butyl Ester

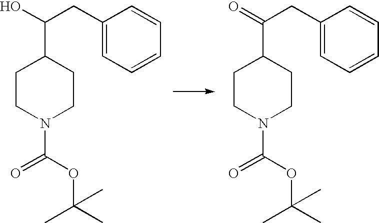

To a mixture of the alcohol of Example 21A (0.241 g, 0.789 mmol), NMO (129 mg, 1.105 mmol) and molecular sieves (1.5 g) in dichloromethane (8 ml) at 0° C. was added TPAP (14 mg, 0.039 mmol). After stirring for 15 hours at room temperature, the mixture was filtered through a pad of silica, washing with diethyl ether, and concentrated. The crude material was purified by silica column chromatography (60% diethyl ether/hexane) to give the title ketone as a clear oil (101 mg, 42%). LC/MS (LCT1): Rₜ 6.93 [M+Na]⁺ 326.

### 21C. 6-(4-Phenylacetylpiperidin-1-yl)-7,9-dihydropurin-8-one

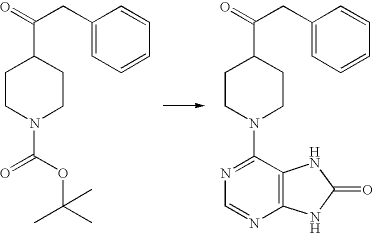

To a solution of the ketone of Example 21B (101 mg, 0.33 mmol) in diethyl ether (3 ml) was added 1M HCl in diethyl ether (3 ml, 3 mmol). After 3 hours, methanol (2 ml) was added. After 2 days the suspension was concentrated. Solid phase extraction on SCX-II acidic resin, eluting with MeOH then 1M NH₃ in MeOH, gave the deprotected piperidine (54 mg, 0.266 mmol).

To a solution of the deprotected piperidine (54 mg, 0.266 mmol) and 6-chloro-7,9-dihydropurin-8-one (45 mg, 0.266 mmol) in n-butanol (2.7 ml) was added triethylamine (185 μl, 1.328 mmol). After refluxing for 24 hours, the solution was cooled, concentrated and the resulting solid triturated with methanol (5 ml) to give the title ketone as a white solid (18 mg, 20%). LC/MS (LCT1): Rₜ 5.84 [M+H]⁺ 338.

### 21D. 6-[4-(1-Amino-2-phenylethyl)piperidin-1-yl]-7,9-dihydropurin-8-one

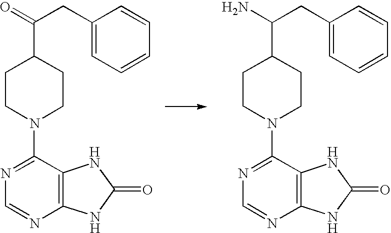

To a solution of the purinone of Example 21C (0.015 g, 0.0445 mmol) in methanol (1 ml) was added ammonium acetate (41 mg, 0.5335 mmol) and sodium cyanoborohydride (11 mg, 0.1778 mmol). After refluxing for 2 days, the suspension was cooled, then purified by solid phase extraction on SCX-II acidic resin, eluting with MeOH then 1M NH₃ in MeOH, which gave the title amine as a 9:1 mixture with starting material. The above reaction sequence was repeated to give the title amine as a white solid (15 mg, 100%). LC/MS (LCT1): Rₜ 4.15 [M+H]⁺ 339.

### Example 22

### 6-(4-[4-(4-Chlorophenyl)-piperidin-4-yl)-phenyl)-9H-purine

### 22A. 4-(4-Bromo-phenyl)-4-(4-chloro-phenyl)-piperidine

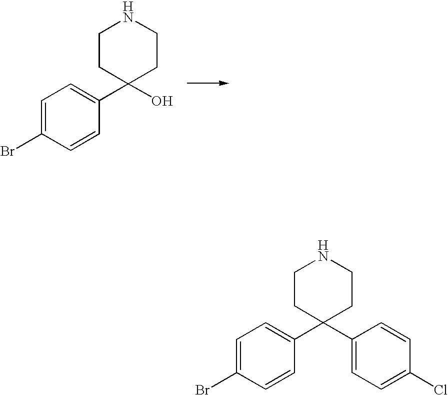

A suspension of 4-(4-bromo-phenyl)-piperidin-4-ol (4.02 g, 15.7 mmol) in chlorobenzene (30 ml) was added dropwise to a suspension of aluminium chloride (7.32 g, 54.9 mmol) in chlorobenzene (10 ml) at 0° C. The reaction mixture was stirred at 0° C. for 2 hours, quenched by addition of ice then methyl t-butyl ether added. After stirring for 1 hour the precipitate was collected by filtration washed with water, methyl t-butyl ether and water to afford the title compound (5.59 g, 92% yield). LC/MS: (PS-B3) Rₜ 3.57 [M+H]⁺ 350, 352.

### 22B. 4-(4-Bromophenyl)-4-(4-chlorophenyl)-piperidine-1-carboxylic Acid Tert-Butyl Ester

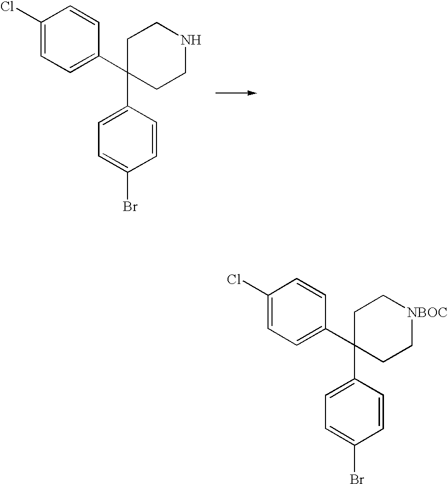

A solution of the 4-(4-bromophenyl)-4-(4-chlorophenyl)-piperidine hydrochloride of Example 22A (1.02 g, 2.64 mmol), triethylamine (2.8 ml, 20 mmol) and di-tert-butyldicarbonate (0.60 g, 2.75 mmol) in dichloromethane (50 ml) was stirred at room temperature for 24 hours. The solution was rinsed with 1M citric acid (50 ml), dried (Na₂SO₄), filtered and concentrated to give a white solid (1.15 g, 97%). ¹H NMR (250 mHz, CDCl₃) δ 1.47 (9H, s), 2.31-2.35 (4H, m), 3.46-3.52 (4H, m), 7.10-7.20 (4H, m), 7.28 (2H, d, J=6 Hz), 7.44 (2H, d, J=6 Hz).

### 22C. 4-(4-(4-Chlorophenyl)-piperidin-4-yl)-phenylboronic acid

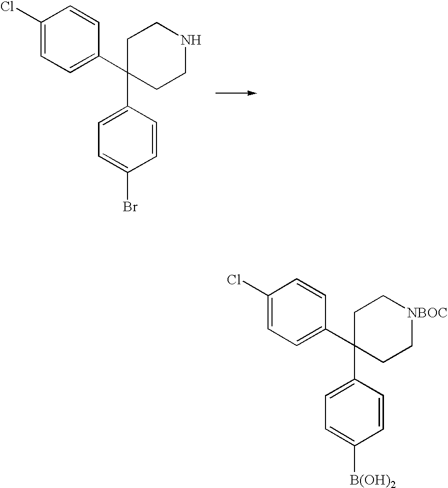

A solution of the 4-(4-bromophenyl)-4-(4-chlorophenyl)-piperidine-1-carboxylic acid tert-butyl ester of Example 22B (0.50 g, 1.11 mmol) and triisopropylborate (0.31 ml, 1.33 mmol) in dry THF (6 ml) was stirred at −78° C. under nitrogen. A solution of n-butyllithium (2M in pentane, 0.67 ml, 1.33 mmol) was added dropwise. The deep red solution was stirred at −78° C. for 30 minutes, becoming pale yellow, then warmed to room temperature and quenched with 1M HCl (aq) (2 ml). The mixture was stirred for 5 minutes then diluted with H₂O (25 ml) and extracted with EtOAc (25 ml). The extract was dried (Na₂SO₄), filtered and concentrated to give a sticky yellow foam. Crystallisation from acetonitrile gave a white solid (0.188 g, 41%).

### 22D. 6-(4-(4-(4-Chlorophenyl)-piperidin-4-yl)-phenyl)-9-(tetrahydropyran-2)-9H-purine

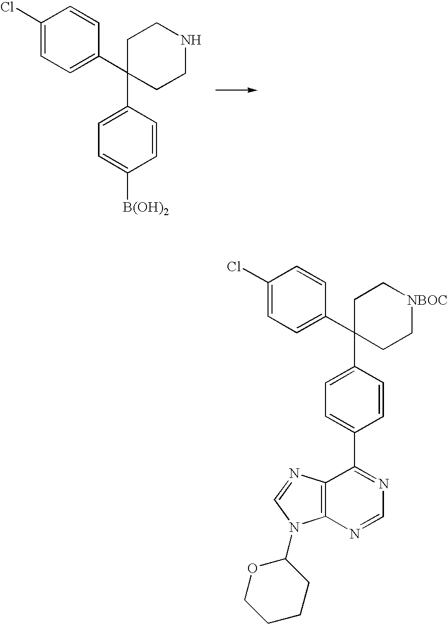

A solution of the boronic acid of Example 22C (0.083 g, 0.2 mmol), 6-chloro-9-(tetrahydropyran-2-yl)-9H-purine (0.050 g, 0.21 mmol), 2M K₂CO₃ (aq) (0.20 ml, 0.40 mmol) and Pd(PPh₃)₄ (0.02 g, 7 mol %) in 1,2-dimethoxyethane (3 ml) was degassed and flushed with nitrogen. The solution was stirred at 85° C. for 16 hours. The solution was partitioned between EtOAc (15 ml) and H₂O (15 ml). The organic layer was dried (Na₂SO₄), filtered and concentrated. Preparative t.l.c., eluting with 50% EtOAc/50% hexane, gave the title product (0.030 g, 26%). LC/MS: (LCT1) Rₜ 8.34 [M+H-THP-tBu]⁺ 434, 436.

### 22E. 6-(4-[4-(4-Chlorophenyl)-piperidin-4-yl)-phenyl)-9H-purine

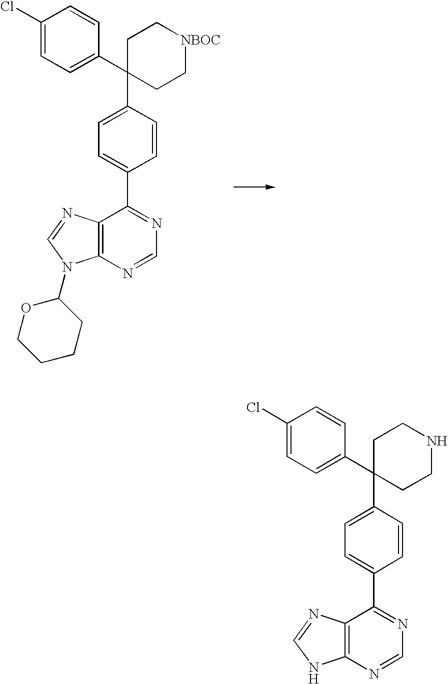

A solution of the protected purine of Example 22D in EtOH (4 ml) with 1M HCl (aq) (2 ml) was stirred at room temperature for 24 hours. Concentrated HCl (3 drops) was added and the mixture was stirred at room temperature for 24 hours, then at 80° C. for 5 hours. The solution was absorbed onto a 5 g SCX-II acidic resin cartridge and eluted with MeOH, then 1M NH₃/MeOH. The basic eluant was concentrated. Trituration and rinsing with diethyl ether gave the product as an off-white solid (0.014 g, 69%). LC/MS: (LCT1) Rₜ 5.00 [M+H]⁺ 390, 392.

### Example 23

### 4-{4-[4-(4-Chloro-phenyl)-piperidin-4-yl]-phenyl}-7H-pyrrolo[2,3-d]pyrimidine

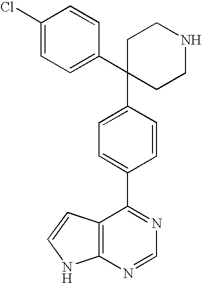

By following a procedure analogous to the method set out in Example 22, the title compound was prepared. LC/MS (LCT1) Rₜ 4.48 (ESI) m/z 389 [M+H]⁺

### Example 24

### C-Phenyl-C-[1-(7H-pyrrolo[2,3-d]pyrimidin-4-yl)-piperidin-4-yl]-methylamine

### 24A. 4-(4-Chlorobenzoyl)piperidine-1-carboxylic Acid Benzyl Ester

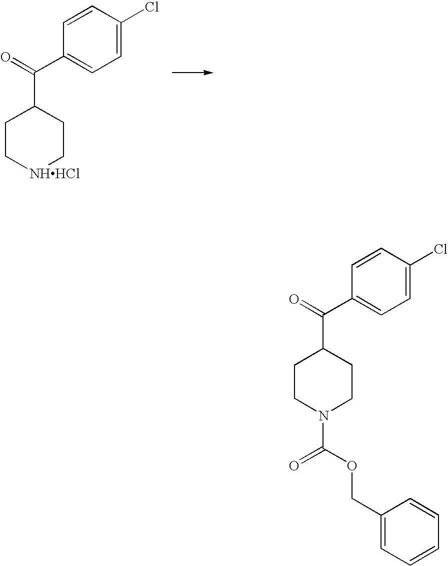

To a mixture of (4-chlorophenyl)piperidin-4-ylmethanone hydrochloride (0.752 g, 2.890 mmol) and triethylamine (1.21 ml, 8.670 mmol) in DCM (20 ml) at 0° C. was added benzyl chloroformate (0.495 ml, 3.468 mmol). After 18 hours at room temperature, the mixture was washed with saturated aqueous sodium bicarbonate (25 ml), then brine (25 ml) before being dried over sodium sulfate and concentrated. The crude material was purified by silica column chromatography (ethyl acetate) to give the ketone as an oil (0.934 g, 100%). LC/MS: (LCT1) Rₜ 7.47 [M+H]⁺ 357.

### 24B. 4-[Amino-(4-chlorophenyl)methyl]piperidine-1-carboxylic Acid Benzyl Ester

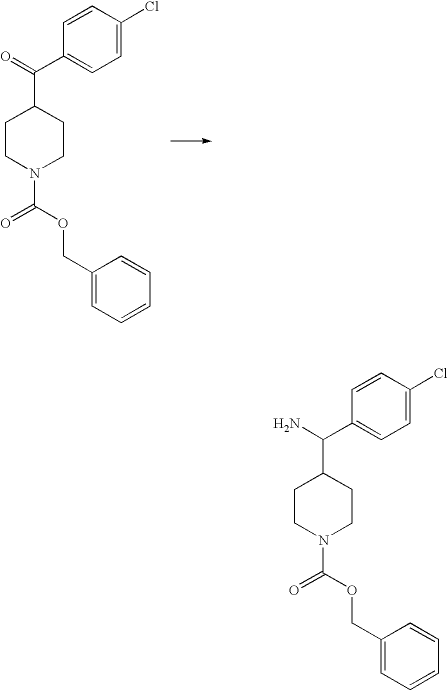

To a mixture of 4-(4-chlorobenzoyl)piperidine-1-carboxylic acid benzyl ester (0.630 g, 1.948 mmol) and ammonium acetate (1.802 g, 23.377 mmol) in methanol (19.5 ml) at room temperature was added sodium cyanoborohydride (0.490 g, 7.792 mmol). After refluxing for 20 hours the mixture was cooled, concentrated and stirred with 1M sodium hydroxide (50 ml). The aqueous phase was extracted with diethyl ether (3×50 ml), with the organic layers being combined, dried over sodium sulphate and concentrated to give the amine as an oil (0.611 g, 97%). LC/MS (LCT1): Rₜ 10.67 [M+H]⁺ 358.

### 24C. 4-[tert-Butoxycarbonylamino(4-chlorophenyl)methyl]piperidine-1-carboxylic Acid Benzyl Ester

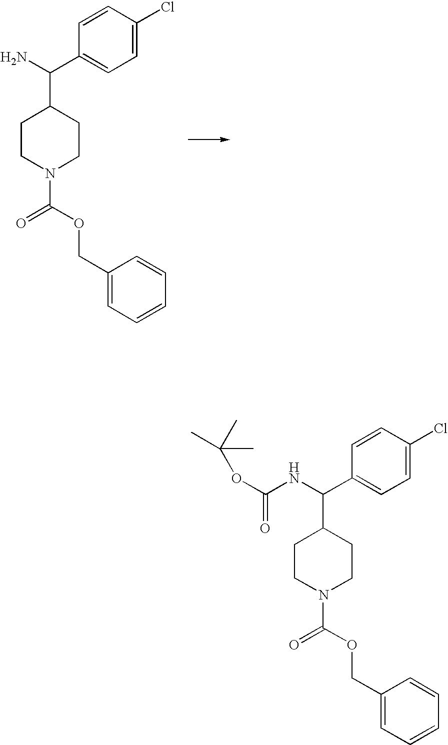

To a solution of 4-[amino-(4-chlorophenyl)methyl]piperidine-1-carboxylic acid benzyl ester (0.611 g, 1.883 mmol) and di-tert-butyl dicarbonate (0.493 g, 2.260 mmol) in acetonitrile (19 ml) at room temperature was added triethylamine (0.788 ml, 5.650 mmol). After 24 hours, the mixture was concentrated, redissolved in ethyl acetate (50 ml) and the organic phase washed with saturated aqueous sodium bicarbonate (50 ml) then brine (50 ml). The organic phase was dried over magnesium sulphate, concentrated and the resulting crude product purified by silica column chromatography (60% diethyl ether in hexanes) to give the protected amine as an oil (0.600 g, 69%). LC/MS (LCT1): Rₜ 7.79 [M+H]⁺ 458.

### 24D. Phenylpiperidin-4-ylmethyl Carbamic Acid Tert-Butyl Ester

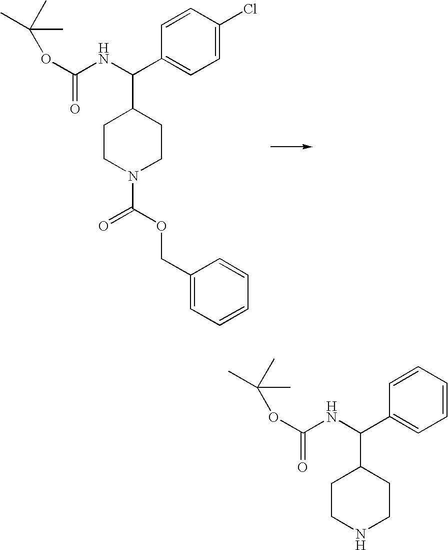

A solution of 4-[tert-butoxycarbonylamino(4-chlorophenyl)methyl]piperidine-1-carboxylic acid benzyl ester (0.217 g, 0.473 mmol) in ethanol (20 ml) was stirred under 1 atmosphere hydrogen pressure over 5% palladium on carbon (40 mg) at room temperature for 1 hour. The reaction mixture was filtered through a pad of celite and the filtrate concentrated to give an oil (0.136 g, 100%). LC/MS (LCT1): Rₜ 4.15 [M+H]⁺ 290.

### 24E. {Phenyl-[1-(7H-pyrrolo[2,3-d]pyrimidin-4-yl)piperidin-4-yl]methyl}carbamic Acid Tert-Butyl Ester

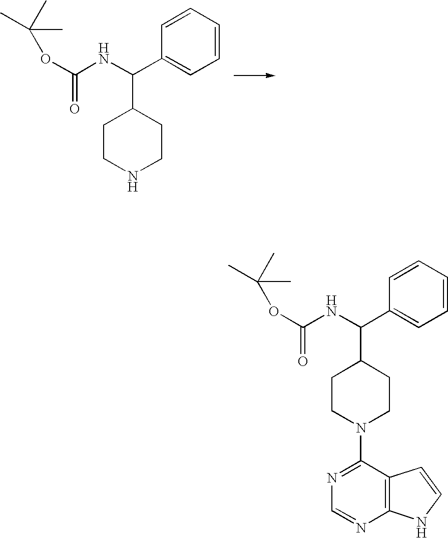

A solution of phenylpiperidin-4-ylmethyl carbamic acid tert-butyl ester (0.070 g, 0.216 mmol), 4-chloro-7H-pyrrolo[2,3-d]pyrimidine (0.033 g, 0.216 mmol) and triethylamine (0.15 ml, 1.078 mmol) in n-butanol (2 ml) was heated at 100° C. for 2 days. The crude mixture was concentrated and purified by silica column chromatography (10% methanol in DCM) to give an oil (52 mg, 59%). ¹H NMR (MeOD) δ 1.20-1.60 (2H, m), 1.43 (9H, s), 1.85-2.15 (2H, m), 2.98-3.16 (2H, m), 4.32-4.36 (1H, m), 4.67-4.88 (2H, m), 6.59-6.60 (1H, m), 7.11-7.13 (1H, m), 8.12 (1H, s).

### 24F. C-Phenyl-C-[1-(7H-pyrrolo[2,3-d]pyrimidin-4-yl)piperidin-4-yl]methylamine

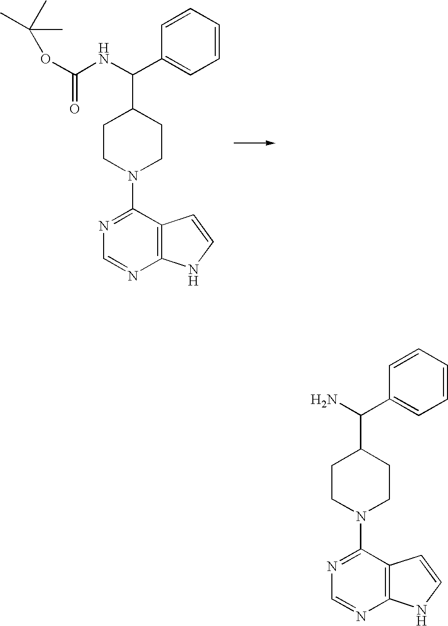

To a solution of {phenyl-[1-(7H-pyrrolo[2,3-d]pyrimidin-4-yl)piperidin-4-yl]methyl}carbamic acid tert-butyl ester (0.050 g, 0.123 mmol) in methanol (3 ml) at room temperature was added 2M hydrochloric acid (3 ml). After 13 hours the mixture was evaporated to dryness. Solid phase extraction on SCX-II acidic resin, eluting with MeOH then 1M NH₃ in MeOH, gave the deprotected amine as a white solid (0.035 g, 92%). LC/MS (LCT1): Rₜ 2.70 [M+H]⁺ 307.

### Example 25

### C-4-Chlorophenyl-C-[1-(7H-pyrrolo[2,3-d]pyrimidin-4-yl)-piperidin-4-yl]-methylamine

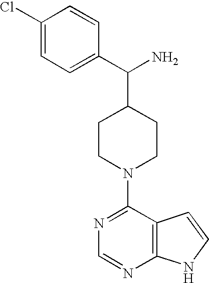

### 25A. 4-(4-Chlorobenzoyl)piperidine-1-carboxylic Acid Tert-Butyl Ester

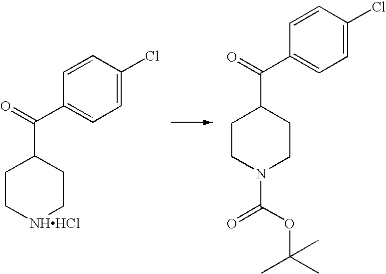

To a mixture of (4-chlorophenyl)piperidin-4-ylmethanone hydrochloride (0.996 g, 3.828 mmol) and triethylamine (2.7 ml, 19.142 mmol) in acetonitrile (15 ml) at room temperature was added di-tert-butyl dicarbonate (1.003 g, 4.594 mmol). After 16 hours at room temperature, the mixture was evaporated to dryness and then partitioned between ethyl acetate (50 ml) and 1M hydrochloric acid (20 ml). The organic phase was separated and washed successively with saturated aqueous sodium bicarbonate (20 ml), then brine (20 ml), before being dried over magnesium sulfate and concentrated to dryness. The crude material was purified by silica column chromatography (60% diethyl ether in hexanes) to give the ketone as an oil (1.116 g, 90%). LC/MS: (LCT1) Rₜ 7.42 [M+H]⁺ 323.

### 25B. 4-[Amino-(4-chlorophenyl)methyl]piperidine-1-carboxylic Acid Tert-Butyl Ester

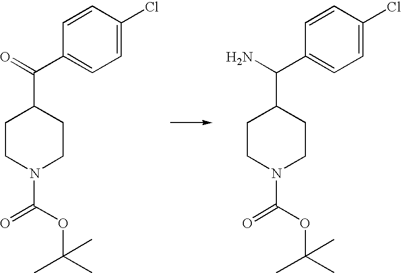

To a mixture of 4-(4-chlorobenzoyl)piperidine-1-carboxylic acid tert-butyl ester (1.116 g, 3.446 mmol) and ammonium acetate (3.188 g, 41.358 mmol) in methanol (34 ml) at room temperature was added sodium cyanoborohydride (0.866 g, 13.786 mmol). After refluxing for 20 hours, the mixture was cooled, concentrated and stirred with 1M sodium hydroxide (100 ml). The aqueous phase was extracted with diethyl ether (3×75 ml), with the organic layers being combined, dried over sodium sulfate and concentrated to dryness. The crude material was purified by silica column chromatography (15% methanol in DCM) to give the amine as an oil (0.913 g, 82%). LC/MS (LCT1): Rₜ 5.56 [M-Boc-NH₂]⁺ 208.

### 25C. C-(4-Chlorophenyl)-C-piperidin-4-ylmethylamine Hydrochloride

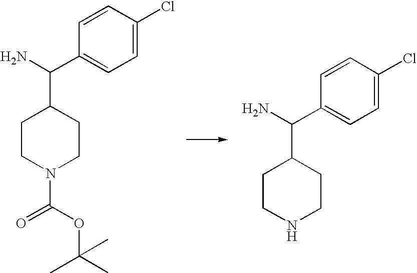

To a solution of 4-[amino-(4-chlorophenyl)methyl]piperidine-1-carboxylic acid tert-butyl ester (0.192 g, 0.591 mmol) in methanol (6 ml) at room temperature was added 2M hydrochloric acid (6 ml). After stirring for 16 hours the solution was evaporated to dryness to give the amine salt as a white foam (0.174 g, 99%). ¹H NMR (MeOD) δ 1.40-1.82 (2H, m), 2.22-2.50 (2H, m), 2.90-3.17 (2H, m), 3.35-3.61 (2H, m), 4.22 (1H, d, 9.5 Hz), 7.53-7.61 (4H, m).

### 25D. C-(4-Chlorophenyl)-C-[1-(7H-pyrrolo[2,3-d]pyrimidin-4-yl)piperidin-4-yl]methylamine

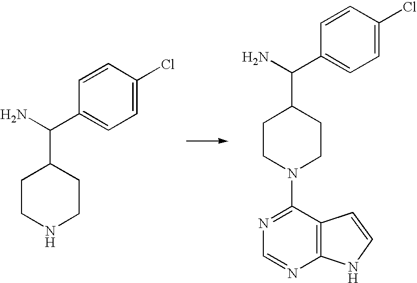

A solution of C-(4-chlorophenyl)-C-piperidin-4-ylmethylamine hydrochloride (0.050 g, 0.168 mmol), 4-chloro-7H-pyrrolo[2,3-d]pyrimidine (0.026 g, 0.168 mmol) and triethylamine (0.117 ml, 0.840 mmol) in n-butanol (1.7 ml) was heated at 100° C. for 2 days. The crude mixture was concentrated, passed through an —NH₂ Isolute column (2 g), concentrated again and purified by silica column chromatography (15% methanol in DCM) to give a off white solid (30 mg, 52%). LC/MS (LCT1): Rₜ 3.35 [M+H]⁺ 341.

### Example 26

### C-(4-Chloro-phenyl)-C-[1-(9H-purin-6-yl)-piperidin-4-yl]-methylamine

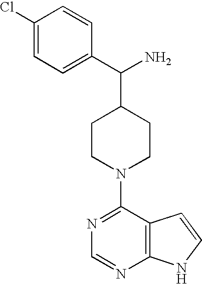

The title compound was prepared by reaction of C-(4-chloro-phenyl)-C-piperidin-4-yl-methylamine (Example 25C) and 6-chloropurine in n-butanol at 100° C. using the method described in Example 25D. LC/MS: (LCT1) Rₜ 4.13 [M+H]⁺ 342.

### Example 27

### 4-{4-[4-(4-Chloro-phenyl)-piperidin-4-yl]-phenyl}-1H-pyrrolo[2,3-b]pyridine

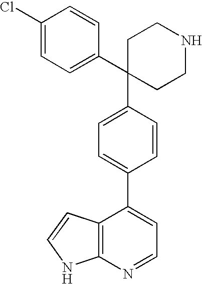

By following a procedure analogous to the method set out in Example 22, the title compound was prepared. LC/MS: (LCT1) Rₜ 4.34 [M+H]⁺ 388.

### Example 28

### C-(4-Chloro-phenyl)-C-[4-(9H-purin-6-yl)-phenyl]-methylamine

### 28A. (4-Bromo-phenyl)-(4-chloro-phenyl)-methanol

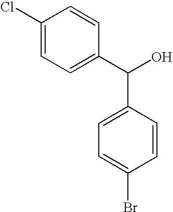

To a cooled (ice bath) solution of 4-bromobenzaldehyde (6.90 g, 37 mmol) in THF (20 ml) was added dropwise 4-chlorophenylmagnesium bromide (40 ml, 1M solution in diethyl ether, 40 mmol). The solution was stirred for 50 minutes, and then saturated ammonium chloride (200 ml) was added, followed by ethyl acetate (250 ml). The layers were separated, and the organic fraction was washed with water (100 ml), then dried, concentrated and purified by flash column chromatography (6:1 hexane:ethyl acetate) to yield the desired product (4.47 g, 41% yield). ¹H NMR (250 MHz, d6-dmso) 3.50 (1H, br s), 5.71 (1H, s), 7.33 (4H, d, J 8.44 Hz), 7.38 (2H, s), 7.51 (2H, d, J 8.46 Hz)

### 28B. 2-[(4-Bromo-phenyl)-(4-chloro-phenyl)-methyl]-isoindole-1,3-dione

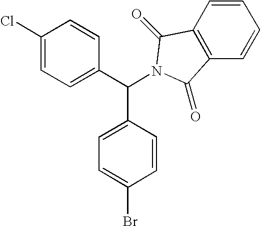

To a solution of (4-Bromo-phenyl)-(4-chloro-phenyl)-methanol (2.30 g, 7.73 mmol), triphenylphosphine (3.42 g, 13.03 mmol) and phthalimide (1.91 g, 12.98 mmol) in THF (60 ml) was added dropwise diisopropylazodicarboxylate (2.40 ml, 12.19 mmol). The solution was stirred for 18 hours, and was then poured into diethyl ether (250 ml). The solution was washed with saturated sodium bicarbonate (2 times 100 ml) and brine (50 ml). The organic fraction was then dried, concentrated and purified by flash column chromatography (6:1 hexane:ethyl acetate) to yield the desired product (0.698 g, 21% yield). LC/MS: (LCT1) Rₜ 8.21 [M+H]⁺ 426.

### 28C. 2-{(4-Chloro-phenyl)-[4-(4,4,5,5-tetramethyl-[1,3,2]dioxaborolan-2-yl)-phenyl]-methyl}-isoindole-1,3-dione

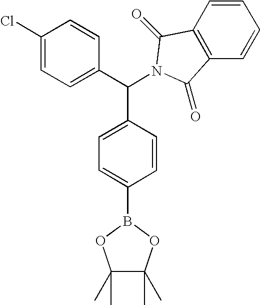

To Pd₂ dba₃ (13 mg, 0.014 mmol) and tricyclohexylphosphine (20 mg, 0.07 mmol) was added dioxane (6 ml). The solution was degassed, and stirred at room temperature for 30 minutes. Bis(pinacolato)diboron (0.256 g, 1 mmol), 2-[(4-Bromo-phenyl)-(4-chloro-phenyl)-methyl]-isoindole-1,3-dione (0.424 g, 1 mmol) and potassium acetate (0.164 g, 1.67 mmol) were then added, and the solution heated at 80° C. for 16 hours. After cooling to room temperature, the solution was poured into ethyl acetate (10 ml) and washed with water (50 ml) and brine (50 ml). The organic layer was dried, concentrated and purified by flash column chromatography (SiO₂, 6:1 hexane:ethyl acetate) to yield the desired product (0.142 g, 30% yield). LC/MS: (LCT1) Rₜ 8.55 [M+Na]⁺ 497.

### 28D. 2-((4-Chloro-phenyl)-{4-[9-(tetrahydro-pyran-2-yl)-9H-purin-6-yl]-phenyl}-methyl)-isoindole-1,3-dione

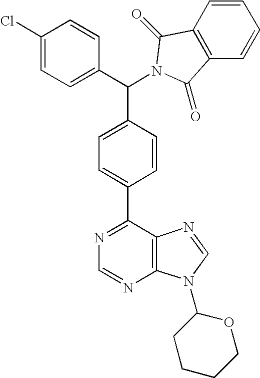

To a solution of 6-chloro-9-(tetrahydro-pyran-2-yl)-9H-purine (0.105 g, 0.44 mmol) and 2-{(4-chloro-phenyl)-[4-(4,4,5,5-tetramethyl-[1,3,2]dioxaborolan-2-yl)-phenyl]-methyl}-isoindole-1,3-dione (0.211 mg, 0.44 mmol) in DME (2 ml) was added PdCl₂(PPh₃)₂. 1M K₂CO₃ (1 ml) was then added and the solution was heated at 80° C. for 18 hours. The mixture was then poured into chloroform/water (100 ml/50 ml), and the layers separated. The product was extracted with chloroform (100 ml) and the combined organic extracts were dried (Na₂SO₄), concentrated, then purified by flash column chromatography (1:1 hexane:ethyl acetate to 1:3 hexane:ethyl acetate) to yield the desired product (0.101 g, 42% yield).

¹H NMR (250 MHz, CDCl₃) 1.60-2.30 (6H, m), 3.82 (1H, dt, J 2.76, 10.99 Hz), 4.15-4.26 (1H, m), 5.85 (1H, dd, J 3.1, 9.8 Hz), 6.77 (1H, s), 7.30-7.41 (4H, m), 7.55 (2H, d, J 8.38 Hz), 7.74 (2H, dd, J 3.04, 5.37 Hz), 7.86 (2H, dd, J 3.1, 5.61 Hz), 8.33 (1H, s), 8.76 (2H, d, J 8.46 Hz), 9.01 (1H, s)

### 28E. C-(4-Chloro-phenyl)-C-{4-[9-(tetrahydro-pyran-2-yl)-9H-purin-6-yl]-phenyl}-methylamine

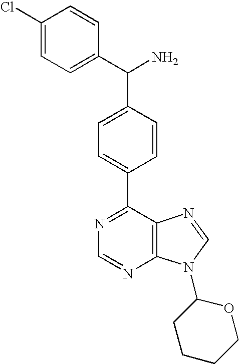

To a solution of 2-((4-chloro-phenyl)-{4-[9-(tetrahydro-pyran-2-yl)-9H-purin-6-yl]-phenyl}-methyl)-isoindole-1,3-dione (0.099 g, 0.18 mmol) in ethanol (6 ml) was added hydrazine hydrate (1 ml). The solution was stirred for 48 hours, then the precipitate was removed by filtration and the filtrate concentrated. The residue obtained was dissolved in methanol, loaded onto an SCX-2 cartridge (2 g), washed with methanol (3 times 5 ml), then eluted with 2M ammonia in methanol (3 times 5 ml). The product obtained was carried forward without further purification.

### 28F. Preparation of C-(4-Chloro-phenyl)-C-[4-(9H-purin-6-yl)-phenyl]-methylamine

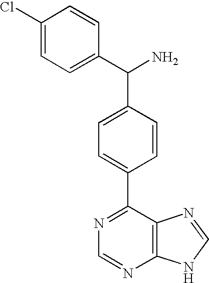

To a solution of C-(4-chloro-phenyl)-C-{4-[9-(tetrahydro-pyran-2-yl)-9H-purin-6-yl]-phenyl}-methylamine (carried forward from previous) in methanol (2 ml) was added 4M HCl in dioxane (2 ml). The mixture was stirred for 18 hours, and then concentrated. The residue was dissolved in methanol and loaded onto a SCX-2 cartridge (2 g) and washed with methanol (3 times 5 ml), then product was eluted with 2M ammonia in methanol (3 times 5 ml). The solution was concentrated to yield the desired product (0.044 g, 73% over 2 steps). LC/MS: (LCT1) Rₜ 4.48 [M+H]⁺ 336.

### Example 29

### C-(4-Chlorophenyl)-C-[1-(1H-pyrrolo[2,3-b]pyridin-4-yl)piperidin-4-yl]methylamine

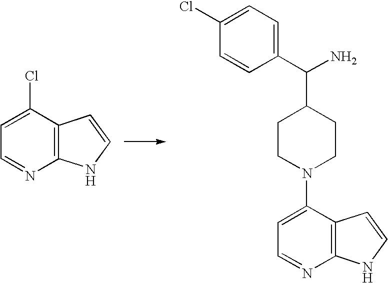

The title compound was prepared using the methods described in Example 25. LC-MS (LCT1) m/z 340 [M+H⁺], Rₜ 2.88 min.

### Example 30

### {2-(4-Chloro-phenyl)-2-[4-(1H-pyrrolo[2,3-b]pyridin-4-yl)-phenyl]-ethyl}-methyl-amine

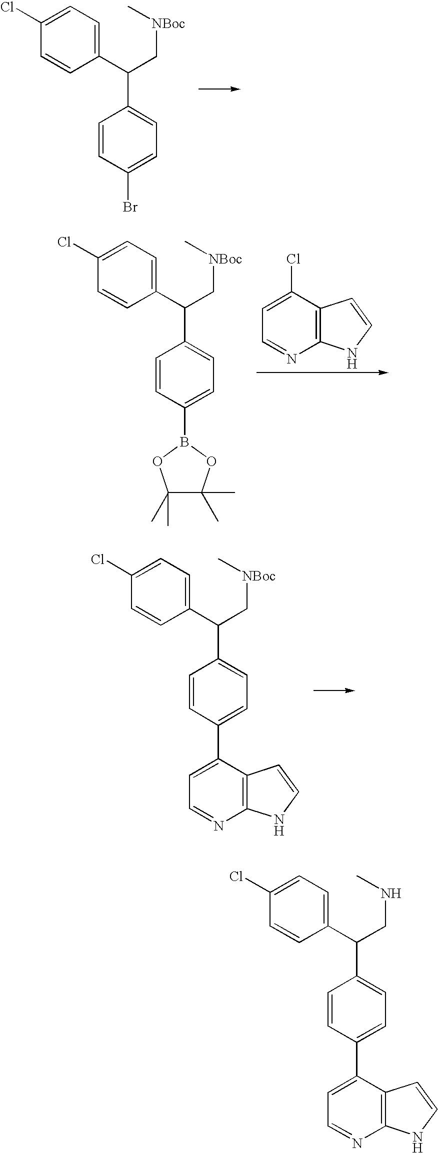

### 30A. {2-(4-Chloro-phenyl)-2-[4-(4,4,5,5-tetramethyl-[1,3,2]dioxaborolan-2-yl)-phenyl]-ethyl}-methyl-carbamic Acid Tert-Butyl Ester

Potassium acetate (218 mg, 2.2 mmol) was added to a degassed solution of [2-(4-bromo-phenyl)-2-(4-chloro-phenyl)-ethyl]-methyl-carbamic acid tert-butyl ester (550 mg, 1.30 mmol) and bis(pinacolato)diboron (338 mg, 1.32 mmol) in dry dioxane (8 ml) at room temperature. This solution was further degassed and flushed with nitrogen (2 cycles). Tricyclohexylphosphine (28 mg, 0.098 mmol) and tris(dibenzylideneacetone)dipalladium (0) (17.6 mg, 0.019 mmol) were added to the reaction mixture. The suspension was further degassed and stirred for 19 hours at 80° C. under nitrogen. After cooling to room temperature, the reaction mixture was partioned between ethyl acetate (50 ml) and water (50 ml). The organic layer was washed with water (2×30 ml), brine (50 ml), dried (Mg₂SO₄), filtered and concentrated. Flash column chromatography on silica, eluting with 15% ethyl acetate in hexane, gave {2-(4-chloro-phenyl)-2-[4-(4,4,5,5-tetramethyl-[1,3,2]dioxaborolan-2-yl)-phenyl]-ethyl}-methyl-carbamic acid tert-butyl ester (131 mg, 0.28 mmol, 21%). LC-MS (LCT2) m/z 494 [M+Na⁺], Rₜ 9.59 min.

### 30B. {2-(4-Chloro-phenyl)-2-[4-(1H-pyrrolo[2,3-b]pyridin-4-yl)-phenyl]-ethyl}-methyl-carbamic Acid Tert-Butyl Ester

A degassed mixture of {2-(4-chloro-phenyl)-2-[4-(4,4,5,5-tetramethyl-[1,3,2]dioxaborolan-2-yl)-phenyl]-ethyl}-methyl-carbamic acid tert-butyl ester (100 mg, 0.21 mmol), 4-chloro-1H-pyrrolo[2,3-b]pyridine (45 mg, 0.29 mmol), 2M aqueous solution of potassium carbonate (0.38 ml, 0.74 mmol), dioxane (4 ml) and Bedford's palladacycle catalyst (Bedford et al, Chem. Commun. 2001, 1540-1541) (13.5 mg, 0.016 mmol) was heated at 100° C. under nitrogen for 17 hours. The solution was cooled and partitioned between dichloromethane (40 ml) and water (40 ml). The aqueous layer was further extracted with dichloromethane (40 ml). The combined organic layers were dried (Na₂SO₄), filtered and concentrated. Flash column chromatography on silica, eluting with 50% ethyl acetate in hexane, gave {2-(4-chloro-phenyl)-2-[4-(1H-pyrrolo[2,3-b]pyridin-4-yl)-phenyl]-ethyl}-methyl-carbamic acid tert-butyl ester (49 mg, 0.11 mmol, 51%). LC-MS (LCT2) m/z 462 [M+H⁺], Rₜ 8.65 min.

### 30C. {2-(4-Chloro-phenyl)-2-[4-(1H-pyrrolo[2,3-b]pyridin-4-yl)-phenyl]-ethyl}-methyl-amine

Trifluoroacetic acid (3.5 ml) was added dropwise to a solution of {2-(4-chloro-phenyl)-2-[4-(1H-pyrrolo[2,3-b]pyridin-4-yl)-phenyl]-ethyl}-methyl-carbamic acid tert-butyl ester (49 mg, 0.11 mmol) in dichloromethane (3.5 ml), cooled in an ice bath. The reaction was allowed to stir at room temperature for 90 minutes. After this period the solvents were concentrated. Purification on SCX-II acid resin, eluting with methanol, then 2M ammonia/methanol, gave {2-(4-chloro-phenyl)-2-[4-(1H-pyrrolo[2,3-b]pyridin-4-yl)-phenyl]-ethyl}-methyl-amine (33 mg, 0.09 mmol, 83%). LC-MS (LCT2) m/z 362 [M+H⁺], Rₜ 4.19 min.

### Example 31

### C-[1-(7H-Pyrrolo[2,3-d]pyrimidin-4-yl)piperidin-3-yl]methylamine

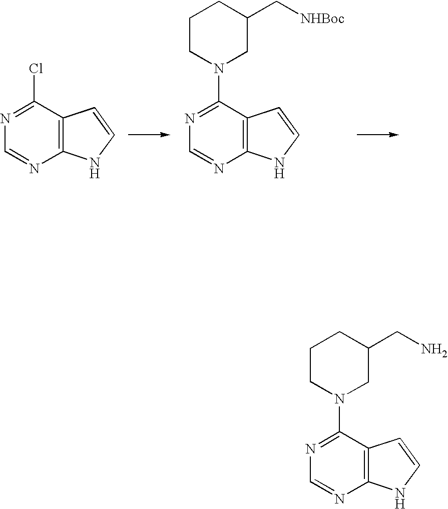

The title compound was prepared using the methods described for Example 18. LC-MS (LCT2) m/z 232 [M+H⁺], Rₜ 0.72 min.

### Example 32

### C-(4-Chlorophenyl)-C-[1-(1H-pyrrolo[2,3-b]pyridin-4-yl)piperidin-4-yl]methylamine

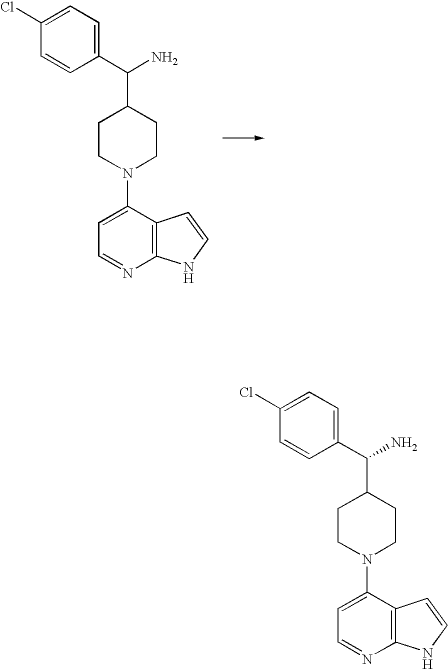

The title compound was prepared by separation of the enantiomers of the product of Example 29 by chiral HPLC using the Agilent chiral preparative conditions set out above. The retention time obtained using the Agilent chiral analytical conditions AS-CA was 33.7. LC/MS subsequently carried out using the PS-A conditions gave a retention time of 1.69 and an [M+H]⁺ value of 341.

### Biological Activity

### Example 33

### Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 50 mM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKA activity (IC₅₀).

Following the protocol described above, the IC₅₀ values of the compounds of Examples 5, 6, 7, 12, 14, 17, 18, 20, 22, 23, 25, 29, 30 and 31 have been found to be less than 10 μM whilst the compound of Example 4 has an IC₅₀ value of less than 150 μM.

### Example 34

### Measurement of PKB Kinase Inhibitory Activity (IC₅₀)

The inhibition of protein kinase B (PKB) activity by compounds can be determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R-K-R-E-R-T-Y-S-F-G-H-H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 μM substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀).

Following the protocol described above, the IC₅₀ values of the compounds of Examples 1 to 7, 10, 12 to 20 and 22 to 32 have been found to be less than 10 μM whilst the compounds of Examples 8, 9 and 11, 21 each have IC₅₀ values of less than 50 μM.

### Example 35

### Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

In particular, compounds of the invention were tested against the PC3 cell line (ATCC Reference: CRL-1435) derived from human prostate adenocarcinoma. Preferred compounds of the invention were found to have IC₅₀ values of less than 30 μM in this assay.

### Pharmaceutical Formulations

### Example 36

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### (iv) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-60. (canceled)

61. A method for treating a disease or condition comprising or arising from abnormal cell growth or abnormally arrested cell death in a mammal, which method comprises administering to the mammal a therapeutically effective amount of a compound having the formula (I): 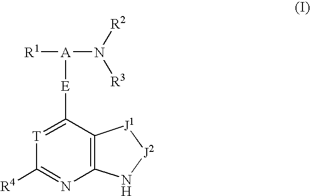
or a salt, solvate, tautomer or N-oxide thereof, wherein
T is N or a group CR⁵;
J₁-J₂ represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen;
halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

62. A method according to claim 61 wherein the compound is a compound of the formula (Ia): 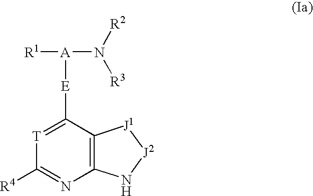
or a salt, solvate, tautomer or N-oxide thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl;
or R² and R³ together with the nitrogen atom to which they are attached form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

63. A compound of the formula (Ib): 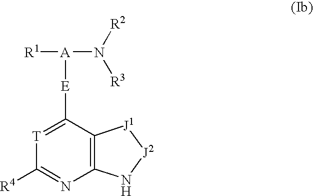
or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
provided that:
(a-i) when J¹-J² is (R⁷)C═C(R⁶) and E is a monocyclic or bicyclic group linked through a nitrogen atom to the ring containing T, then A contains no oxo substituent;
(a-ii) E is other than an unsubstituted or substituted indole group;
(a-iii) when J¹-J² is N═CH, then E-A(R¹)—NR²R³ is other than a group —S—(CH₂)₃—CONH₂ or —S—(CH₂)₃—CN;
(a-iv) when J¹-J² is CH═N, then E-A(R¹)—NR²R³ is other than a group —NH—(CH₂), N(CH₂CH₃)₂ where n is 2 or 3; and
(a-v) when J¹-J² is N═CH, then E-A(R¹)—NR²R³ is other than a group —NH—(CH₂)₂NH₂ or —NH—(CH₂)₂—N(CH₃)₂.

64. A compound according to claim 63 having the formula (Ic): 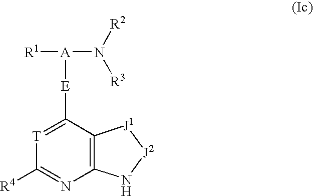
or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from fluorine and hydroxy, provided that the hydroxy group when present is not located at a carbon atom a with respect to the NR²R³ group;
E is a monocyclic carbocyclic or heterocyclic group;
R¹ is an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

65. A compound according to claim 64 wherein E is selected from phenyl and piperidine groups.

66. A compound according to claim 65 wherein E is unsubstituted.

67. A compound according to claim 63 wherein R⁴ is hydrogen.

68. A compound according to claim 63 wherein T is N or CR⁵ where R⁵ is hydrogen.

69. A compound according to claim 63 wherein each of R⁶, R⁷ and R⁸ when present is hydrogen.

70. A compound according to claim 63 wherein the linker group A has a maximum chain length of 3 atoms extending between R¹ and NR²R³.

71. A compound according to claim 63 wherein the linker group A has a maximum chain length of 4 atoms, extending between E and NR²R³.

72. A compound according to claim 63 wherein the linker group A has a chain length of 1, 2 or 3 atoms extending between R¹ and NR²R³ and a chain length of 1, 2 or 3 atoms extending between E and NR²R³.

73. A compound according to claim 63 wherein the linker group A has an all-carbon skeleton.

74. A compound according to claim 63 wherein R¹ is an aryl or heteroaryl group.

75. A compound according to claim 63 wherein R¹ is selected from unsubstituted or substituted phenyl, naphthyl, thienyl, furan, pyrimidine and pyridine groups.

76. A compound according to claim 75 wherein R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

77. A compound according to claim 63 wherein R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group.

78. A compound according to claim 77 wherein R² and R³ are independently selected from hydrogen, unsubstituted C₁₋₄ hydrocarbyl and unsubstituted C₁₋₄ acyl.

79. A compound according to claim 78 wherein R² and R³ are independently selected from hydrogen and methyl.

80. A compound according to claim 79 wherein NR²R³ is an amino group or a methylamino group.

81. A compound according to claim 63 wherein J¹-J² is selected from N═CH, HC═N, HN—C(O) and CH═CH.

82. A compound according to claim 63 having the formula (II): 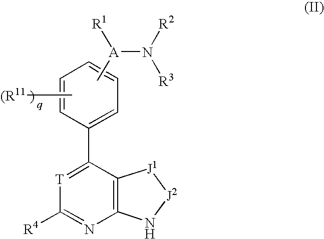
or being a salt, solvate, tautomer or N-oxide thereof;
wherein the group A is attached to the meta or para position of the benzene ring, q is 0-4; T, J¹-J², A, R¹, R², R³ and R⁴ are as defined in claim 3; and R¹¹ is selected from hydroxy; CH₂CN; halogen; trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

83. A compound according to claim 63 having the formula (III): 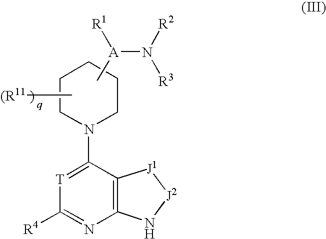
or being a salt, solvate, tautomer or N-oxide thereof;
wherein the group A is attached to the 3-position or 4-position of the piperidine ring, q is 0-4; T, J¹-J², A, R¹, R², R³ and R⁴ are as defined in claim 3 and R¹¹ is selected from hydroxy; CH₂CN; oxo; halogen; trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

84. A compound as defined in claim 63 in the form of a salt, solvate or N-oxide.

85. A method of modulating a cellular process by inhibiting the activity of a protein kinase B or protein kinase A using a compound as defined in claim 61.

86. A pharmaceutical composition comprising a compound as defined in claim 63 and a pharmaceutically acceptable carrier.

87. A method for the diagnosis and treatment of a disease state or condition mediated by protein kinase B or protein kinase A, which method comprises (i) screening a patient to determine whether a disease or condition from which the patient is or may be suffering is one which would be susceptible to treatment with a compound having activity against protein kinase B or protein kinase A; and (ii) where it is indicated that the disease or condition from which the patient is thus susceptible, thereafter administering to the patient a compound as defined in claim 61.

88. A process for the preparation of a compound as defined in claim 63, which process comprises:
(a) when E is an aryl or heteroaryl group, the reaction of a compound of the formula (X) with a compound of the formula (XI), where (X) and (XI) may be suitably protected and wherein one of the groups X and Y is chlorine, bromine or iodine or a trifluoromethanesulphonate (triflate) group, and the other one of the groups X and Y is a boronate residue, for example a boronate ester or boronic acid residue, 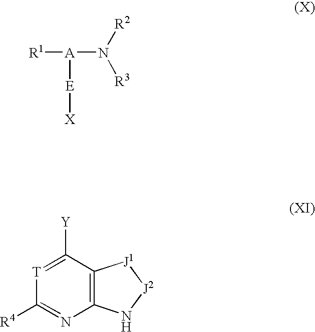
in the presence of a palladium catalyst;
(b) the reductive amination of an aldehyde compound of the formula (XVI): 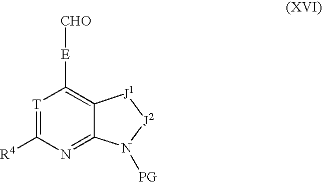
where PG is a protecting group, with an amine of the formula HNR²R³ in the presence of a reducing agent;
(c) the reaction of the aldehyde (XVI) with tert-butyl sulphinamide in the presence of a dehydrating agent to give an intermediate tert-butyl sulphinylimine (not shown) followed by reaction with a Grignard reagent R¹—MgBr to give a tert-butyl sulphinylamino derivative (XVII): 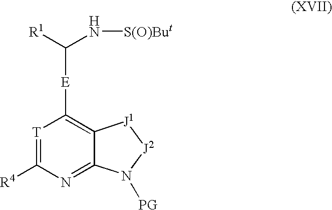
and thereafter removing the S(O)Bu^{t} group by hydrolysis and removing the protecting group PG;
(d) when ANR²R³ is CHCH₂CN or CHCH₂CH₂NR²R³, the reaction of the aldehyde (XVI) with malononitrile or ethylcyanoacetate in the presence of a base to give an intermediate cyanoacrylate derivative followed by reaction of the cyanoacrylate derivative with a Grignard reagent R¹—MgBr and subsequent hydrolysis and decarboxylation;
(e) when E is a non-aromatic cyclic group or an acyclic group and is linked to the bicyclic group by a nitrogen atom, the reaction of a compound of the formula (XXIX) with an amine compound H₂N-G or a compound of the formula (XXX) or a protected derivative thereof, where G is as defined in any one of the preceding claims and the ring E represents a cyclic group E containing a nucleophilic NH group as a ring member; 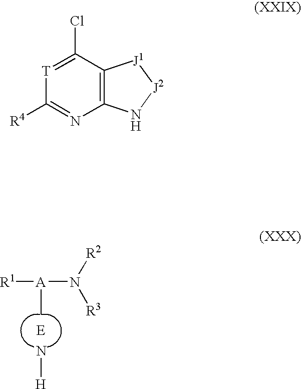
and optionally thereafter:
(f) converting one compound of the formula (I) into another compound of the formula (I).
